(19) United States
(12) Patent Application Publication
(10) Pub. No.: US 2018/0251725 A1
(43) Pub. Date: Sep. 6, 2018

(54) SYSTEMS AND METHODS FOR ENRICHING A BACTERIAL STRAIN FROM A TARGET BACTERIAL SYSTEM

(71) Applicant: NuByiota LLC, Teaneck, NJ (US)

(72) Inventor: Emma Allen-Vercoe, Guelph (CA)

(21) Appl. No.: 15/754,742

(22) PCT Filed: Aug. 24, 2016
(86) PCT No.: PCT/US16/48317

§ 371 Date: Feb. 23, 2018

Related U.S. Application Data
(60) Provisional application No. 62/209,135, filed on Aug. 24, 2015.

(51) Int. Cl. C12N 1/20; A61K 35/38; A61K 35/74

(52) U.S. Cl. CPC C12N 1/20; A61K 35/38; C12N 2500/76; C12N 2500/34; A61K 35/74

(57) ABSTRACT

The present invention is a method of enriching at least one bacterial species from a target bacterial system, including: culturing the target bacterial ecosystem in a culture media in a single-stage chemostat under the following conditions: (i) a system retention time of about 5 to about 290 hours, (ii) a temperature of about 37° C., (iii) a pH of about 6.8 to 7, and (iv) maintenance of Establish anaerobic conditions to the chemostat for a time sufficient to enrich the at least one bacterial species; where the culture media comprises a prepared starch substrate, and where the target bacterial system is a fecal derived sample obtained from a patient that has not been treated with an antibiotic for at least 6 months.

### RELATED APPLICATIONS

This application claims the priority of U.S. provisional application U.S. Patent Appln. No. 62/209,135; filed Aug. 24, 2015; entitled “INFLUENCE OF NOVEL MAIZE STARCHES ON HUMAN COLONIC MICROBIAL FERMENTATION,” which is incorporated herein by reference in its entirety for all purposes.

### FIELD OF THE INVENTION

The field of the invention relates to therapies for treating gastrointestinal disorders. In particular, the present invention provides systems and methods for enriching at least one bacterial strain from a fecal-derived bacterial population. These systems and methods can be used as therapies for treating gastrointestinal disorders.

### BACKGROUND OF THE INVENTION

The various microbes that inhabit the surfaces of the human body both internally and externally compose what is known as the human microbiota. These microbes are estimated to outnumber human somatic cells ten to one, and contain over 150 times more genes than that of the host genome, providing the host with the ability to perform various functions without evolving the required genes independently. The human gastrointestinal tract (GIT) is one of the most heavily populated ecosystems on the planet containing >1014 microbial cells, representing approximately 500-1000 unique species, with the densest populations located in the colon. Infants are born with a sterile GIT; colonization commences during the delivery process and progresses towards a fully developed, complex, and stable microbiota by weaning. The development of the microbiota is a complex process affected by intrinsic factors; intestinal pH, immune responses, and other genetic determinants. Environmental factors such as drugs, diet, maternal microbiota and method of delivery further shape the development, as do host-microbe interactions with receptors and signalling molecules.

It has been proposed that the function of the gut microbiota resembles that of a “virtual organ” having both local and systemic effects. Examples include: metabolism of nutrients such as polysaccharides that are either indigestible or inaccessible by host enzymes, providing additional energy and synthesizing vitamins for absorption. Microbial fermentation has been shown to account for approximately 10% of the daily energy supply in western diets. The gut microbiota is also responsible for the proper development of the gut epithelium, a physical barrier between the intestinal lumen and the body's immune cells. This is accomplished by mediating proper glycosylation of surface proteins, development of microvilli, and regulating cell turn over. Colonization resistance via competition for nutrients and adherence sites, production of anti-microbials and the modulation of the intestinal environment (e.g. lowering of pH) protect the host from potential pathogens. Finally the microbiota contributes to the maturation of the immune system by providing essential stimuli and inducing tolerance mechanisms.

As previously stated, the development and maintenance of our colonic flora is multi-factorial, with diet being the simplest factor to modulate. Controlled dietary adjustment may provide a possible means of therapeutic intervention for a variety of health concerns through the use of selected prebiotics and probiotics. Long term dietary trends indicate a strong correlation between diet and enterotype; Western diets typically high in fat/protein were linked to one enterotype, while a second enterotype was associated with diets high in carbohydrates and simple sugars typical of agrarian societies. A short term feeding study demonstrated that changes between high fat/low fiber and low fat/high fiber diets induced rapid microbial changes detectible within 24 hours. The magnitude of these changes was small and unable to cause enterotype switching; therefore enterotypes may be influenced by long term, but not short-term dietary trends.

Among the many dietary substrates that have been studied to date resistant starch, a fraction of starch indigestible by mammalian enzymes, stands out as an important modulator of the gut microbiota. Increasing the amount and type of resistant starches in the diet therefore represents a feasible and straightforward way to beneficially modulate the gut microbiota. In order to do this in a targeted fashion, it will be important to first understand the impact various forms of resistant starch has on the gut microbiota.

### BRIEF DESCRIPTION OF THE FIGURES

The present invention will be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular components.

In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

FIGS. 1A-F shows sequence comparisons employed in the methods according to some embodiments of the present invention.

FIGS. 2A-F shows sequence alignment diagrams employed in the methods according to some embodiments of the present invention.

FIGS. 3A-C shows some scatter plots used for comparisons employed in the methods according to some embodiments of the present invention.

FIGS. 4A-D shows some comparisons for identifying species matches employed in the methods according to some embodiments of the present invention.

FIGS. 5A-5H show KEGG pathway maps used to identify metabolic pathways employed in the methods according to some embodiments of the present invention.

FIGS. 6A-6H show a metabolic pathway map of one or more species employed in the methods according to some embodiments of the present invention.

FIGS. 7A-7Q show metabolic pathway maps employed in the methods according to some embodiments of the present invention.

FIGS. 8A-8H show a pathway map to compare 22 species employed in the methods according to some embodiments of the present invention.

FIGS. 9 and 10 show a single-stage chemostat vessel employed in the methods according to some embodiments of the present invention.

FIG. 11 shows denaturing gradient gel electrophoresis (DGGE) profiles of six starch substrates employed in the methods according to some embodiments of the present invention.

FIGS. 12A-C show community dynamics of chemostat runs seeded with feces from three healthy donors as employed in the methods according to some embodiments of the present invention.

FIGS. 13A-C show dendrograms based on Pearson and unweighted pair group with mathematical averages (UPGMA) correlation of the DGGE profiles comparing microbial communities as employed in the methods according to some embodiments of the present invention.

FIGS. 14A-C show non-metric multidimensional scaling (NMDS) plots as employed in the methods according to some embodiments of the present invention.

FIG. 15 shows a dendrogram based on Pearson and UPGMA correlation of the DGGE profiles comparing microbial communities plots as employed in the methods according to some embodiments of the present invention.

FIG. 16 shows NMDS plots from similarity matrix generated from Pearson and UPGMA correlation of the DGGE profiles comparing microbial communities as employed in the methods according to some embodiments of the present invention.

FIG. 17 shows a dendrogram based on Pearson and UPGMA correlation of the DGGE profiles comparing microbial communities as employed in the methods according to some embodiments of the present invention.

FIG. 18 shows NMDS plots from similarity matrix generated from Pearson and UPGMA correlation of the DGGE profiles comparing microbial communities as employed in the methods according to some embodiments of the present invention.

FIG. 19 shows Dendrogram based on Pearson and UPGMA correlation of the DGGE profiles comparing microbial communities as employed in the methods according to some embodiments of the present invention.

FIG. 20 shows NMDS plots from similarity matrix generated from Pearson and UPGMA correlation of the DGGE profiles comparing microbial communities as employed in the methods according to some embodiments of the present invention.

FIG. 21 shows Dendrogram based on Pearson and UPGMA correlation of the DGGE profiles comparing microbial communities as employed in the methods according to some embodiments of the present invention.

FIGS. 22A-F show principal component analysis data as employed in the methods according to some embodiments of the present invention.

FIG. 23 shows principal component analysis data as employed in the methods according to some embodiments of the present invention.

FIG. 24 shows a flowchart of experimental design as employed in the methods according to some embodiments of the present invention.

FIGS. 25A and 25B show DGGE analysis of the in vitro feeding trial as employed in the methods according to some embodiments of the present invention.

FIGS. 26A and 26B show DGGE analysis of the in vitro feeding trial as employed in the methods according to some embodiments of the present invention.

FIGS. 27A-E show dendrograms based on Pearson and UPGMA correlation of the DGGE profiles comparing microbial communities as employed in the methods according to some embodiments of the present invention.

FIGS. 28A-E show NMDS plots of similarity matrixes generated from Pearson and UPGMA correlation of the DGGE profiles comparing microbial communities as employed in the methods according to some embodiments of the present invention.

FIGS. 29A-E show dendrograms based on Pearson and UPGMA correlation of the DGGE profiles comparing microbial communities as employed in the methods according to some embodiments of the present invention.

FIGS. 30A-E show NMDS plots of similarity matrixes generated from Pearson and UPGMA correlation of the DGGE profiles comparing microbial communities as employed in the methods according to some embodiments of the present invention.

FIGS. 31A-E show results from a fermentation study as employed in the methods according to some embodiments of the present invention.

FIGS. 32A-E show NMDS plots of similarity matrixes generated from Pearson and UPGMA correlation of the DGGE profiles comparing microbial communities as employed in the methods according to some embodiments of the present invention.

FIGS. 33A-E show dendrograms based on Pearson and UPGMA correlation of the DGGE profiles comparing microbial communities as employed in the methods according to some embodiments of the present invention.

FIG. 34 shows total ion chromatograms from a fermentation experiment as employed in the methods according to some embodiments of the present invention.

### SUMMARY OF INVENTION

In some embodiments, the present invention is a method of enriching at least one bacterial species from a target bacterial system, comprising:

culturing the target bacterial ecosystem in a culture media in a single-stage chemostat under the following conditions: (i) a system retention time of about 5 to about 290 hours, (ii) a temperature of about 37° C., (iii) a pH of about 6.8 to 7, and (iv) maintenance of anaerobic conditions to the chemostat for a time sufficient to enrich the at least one bacterial species;
wherein the culture media comprises a prepared starch substrate, and
wherein the target bacterial system is a fecal derived sample obtained from a patient that has not been treated with an antibiotic for at least 6 months.

In some embodiments, the prepared starch substrate comprises: a maize substrate, a corn substrate, a wheat substrate, a barley substrate, a legume substrate, an oat substrate, or any combination thereof. In some embodiments, the at least one bacterial species comprises: a Bacteroides spp., an Atopobium spp., Ruminococcus bromii, Lactobacillus gasseri, and Parabacteroides distasonis. In some embodiments, the prepared starch substrate is a maize substrate. In some embodiments, the patient has not been treated with an antibiotic for at least 1 year. In some embodiments, the system retention time is between about 20 to 70 hours.

In some embodiments, the present invention is a method of enriching at least one bacterial strain from a target bacterial system, comprising:

culturing the target bacterial ecosystem in a culture media in a single-stage chemostat under the following conditions: (i) a system retention time of about 5 to about 290 hours, (ii) a temperature of about 37° C., (iii) a pH of about 6.8 to 7, and (iv) maintenance of anaerobic conditions to the chemostat for a time sufficient to enrich the at least one bacterial strain;
wherein the culture media comprises a prepared starch substrate, and
wherein the target bacterial system is a fecal derived sample obtained from a patient that has not been treated with an antibiotic for at least 6 months.

In some embodiments, the prepared starch substrate comprises: a maize substrate, a corn substrate, a wheat substrate, a barley substrate, a legume substrate, an oat substrate, or any combination thereof. In some embodiments, the at least one bacterial strain comprises: a Bacteroides spp., an Atopobium spp., Ruminococcus bromii, Lactobacillus gasseri, and Parabacteroides distasonis. In some embodiments, the prepared starch substrate is a maize substrate. In some embodiments, the patient has not been treated with an antibiotic for at least 1 year. In some embodiments, the system retention time is between about 20 to 70 hours.

### DETAILED DESCRIPTION OF THE INVENTION

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the description, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the term “dysbiosis” refers to an imbalance of a subject's gut microbiome.

As used herein, the term “microbiome” refers to all the microbes in a community. As a non-limiting example, the human gut microbiome includes all of the microbes in the human's gut.

As used herein, the term “chemotherapy-related dysbiosis” refers to any intervention used to target a subject's particular disease which leads to an imbalance of the subject's gut microbiome.

As used herein, the term “fecal bacteriotherapy” refers to a treatment in which donor stool is infused into the intestine of the recipient to re-establish normal bacterial microbiota. Fecal bacteriotherapy has shown promising results in preliminary studies with close to a 90% success rate in 100 patient cases published thus far. Without being bound by theory, it is believed to work through breaking the cycle of repetitive antibiotic use, re-establishing a balanced ecosystem that represses the growth of C. difficile.

As used herein, the term “keystone species” are species of bacteria which are consistently found in human stool samples.

As used herein, the term “OTU” refers to an operational taxonomic unit, defining a species, or a group of species via similarities in nucleic acid sequences, including, but not limited to 16S rRNA sequences.

In some embodiments, the present invention is a method of enriching at least one bacterial strain from a target bacterial system, comprising:

culturing the target bacterial ecosystem in a culture media in a single-stage chemostat under the following conditions: (i) a system retention time of about 5 to about 290 hours, (ii) a temperature of about 37° C., (iii) a pH of about 6.8 to 7, and (iv) maintenance of anaerobic conditions to the chemostat for a time sufficient to enrich the at least one bacterial strain;
wherein the culture media comprises a prepared starch substrate, and
wherein the target bacterial system is a fecal derived sample obtained from a patient that has not been treated with an antibiotic for at least 6 months.

In some embodiments, the prepared starch substrate comprises: a maize substrate, a corn substrate, a wheat substrate, a barley substrate, a legume substrate, an oat substrate, or any combination thereof. In some embodiments, the at least one bacterial strain comprises: a Bacteroides spp., an Atopobium spp., Ruminococcus bromii, Lactobacillus gasseri, and Parabacteroides distasonis. In some embodiments, the prepared starch substrate is a maize substrate. In some embodiments, the patient has not been treated with an antibiotic for at least 1 year. In some embodiments, the system retention time is between about 20 to 70 hours.

In some embodiments, the system retention time is between about 5 to 250 hours. In some embodiments, the system retention time is between about 5 to 200 hours. In some embodiments, the system retention time is between about 5 to 150 hours. In some embodiments, the system retention time is between about 5 to 100 hours. In some embodiments, the system retention time is between about 5 to 90 hours. In some embodiments, the system retention time is between about 5 to 80 hours. In some embodiments, the system retention time is between about 5 to 70 hours. In some embodiments, the system retention time is between about 5 to 60 hours. In some embodiments, the system retention time is between about 5 to 50 hours. In some embodiments, the system retention time is between about 5 to 40 hours. In some embodiments, the system retention time is between about 5 to 30 hours. In some embodiments, the system retention time is between about 5 to 20 hours.

In some embodiments, the system retention time is between about 20 to 250 hours. In some embodiments, the system retention time is between about 30 to 250 hours. In some embodiments, the system retention time is between about 40 to 250 hours. In some embodiments, the system retention time is between about 50 to 250 hours. In some embodiments, the system retention time is between about 60 to 250 hours. In some embodiments, the system retention time is between about 70 to 250 hours. In some embodiments, the system retention time is between about 80 to 250 hours. In some embodiments, the system retention time is between about 90 to 250 hours. In some embodiments, the system retention time is between about 100 to 250 hours. In some embodiments, the system retention time is between about 150 to 250 hours. In some embodiments, the system retention time is between about 200 to 250 hours.

In some embodiments, the system retention time is between about 20 to 200 hours. In some embodiments, the system retention time is between about 50 to 150 hours. In some embodiments, the system retention time is between about 50 to 100 hours. In some embodiments, the system retention time is between about 100 to 150 hours.

This study evaluated differences between in vitro, small-scale batch fermentations of 6 different maize starch substrates by human gut microbiota. Stable fecal communities from 3 donors, grown in chemostats modeling the distal colon, were used as fecal inocula in batch fermentations. Denaturing gradient gel electrophoresis (DGGE) and gas chromatography-mass spectrometry (GC-MS) were used to assess changes in community structure and production of metabolites such as short chain fatty acids (SCFAs) for each fecal community. The tested starch substrates promoted unique changes to the fecal communities of each donor, suggesting that an individual's fecal microbiota ferments various starch substrates in different ways. GC-MS data support these donor specific results, indicating a significant production of butyrate for all 3 donor's microbiota, while significant increases in pentanoic and propanoic acid were observed in just one. Although community profiles exhibited changes according to starch substrate fermented, no clear differences in metabolites were observed.

List of Abbreviations

ACF Aberrant crypt

foci ae- amylose extender

AMG Amyloglucosidase

BMI Body mass index

DGGE Denaturing gradient gel electrophoresis

DP Degrees of polymerization

DVB/CAR/PDMS Divinylbenzene/Carboxen/Polydimethylsiloxane

EI Electron impact

EMA Ethidium monoazide

EPIC European Prospective Investigation into Cancer and Nutrition

FAAb Fastidious anaerobe agar supplemented with 5% defibrinated sheep blood

GBSS Granule bound starch synthase

GC Gas chromatography

GC-MS Gas chromatography coupled to mass spectrometry

GIT Gastrointestinal tract

GOPOD Glucose oxidase-peroxidase reagent

HAMS High amylose maize starch

HAMSB Butyrylated high amylose maize starch

HDL High density lipoprotein

HPLC High pressure liquid chromatography

LAMS Low amylose maize starches

MDS Multidimensional scaling

NMDS Non-metric multidimensional scaling

NMR Nuclear magnetic resonance

OPLS-DA Orthogonal projections to latent structures discriminant analysis

PBS Phosphate buffered saline

PC Principal component

PCA Principal component analysis

PTFE Polytetrafluoroethylene

RDS Rapidly digestible starch

RS Resistant starch

SBEIIb Starch branching enzyme IIb

SCFA Short chain fatty acids

SDS Slowly digestible starch

SI Similarity index

SPME Solid phase microextraction

SS Soluble starch SSIIa

Starch synthase IIa

su2 sugary2

TAE Tris-acetate-EDTA

TS Total starch

UPGMA Unweighted pair group with mathematical averages

VIP Variable influence on projection

VOC Volatile organic compounds

wx waxy

Δt Rate of change

### Fecal-Derived Bacterial Populations

In some embodiments, the present invention provides a method wherein the method treats a subject having a dysbiosis comprising determining a first metabolic profile of a subject having a dysbiosis; changing the first metabolic profile of the subject to a second metabolic profile of the subject by administering to the subject a therapeutically effective amount or an amount needed to colonize the subject and alter the first metabolic profile to the second metabolic profile of at least one bacterial strain selected from the group consisting of: Acidaminococcus intestinalis 14LG, Bacteriodes ovatus 5MM, Bifidobacterium adolescentis 20MRS, Bifidobacterium longum, Blautia sp. 27FM, Clostridium sp. 21FAA, Collinsella aerofaciens, Escherichia coli 3FM4i, Eubacterium desmolans 48FAA, Eubacterium eligens F1FAA, Eubacterium limosum 13LG, Faecalibacterum prausnitzii 40FAA, Lachnospira pectinoshiza 34FAA, Lactobacillus casei 25MRS, Parabacteroides distasonis 5FM, Roseburia faecalis 39FAA, Roseburia intestinalis 31FAA, Ruminococcus sp. 11FM, Ruminococcus species, Ruminococcus torques 30FAA; and any combination thereof; and treating the subject so as to result in sufficient colonization of the subject with the at least one bacterial strain; where the first metabolic profile is a consequence of the dysbiosis, and where the second metabolic profile treats the subject having the dysbiosis.

In some embodiments, the method further includes administering to the subject a therapeutically effective amount of at least one bacterial strain selected from the group consisting of: 16-6-I 21 FAA 92% Clostridium cocleatum; 16-6-I 2 MRS 95% Blautia luti; 16-6-I 34 FAA 95% Lachnospira pectinoschiza; 32-64 30 D6 FAA 96% Clostridium glycyrrhizinilyticum; 32-6-I 28 D6 FAA 94% Clostridium lactatifermentans; and any combination thereof.

In some embodiments, the dysbiosis is associated with gastrointestinal inflammation. In some embodiments, the gastrointestinal inflammation is an inflammatory bowel disease, irritable bowel syndrome, diverticular disease, ulcerative colitis, Crohn's disease, or indeterminate colitis.

In some embodiments, the dysbiosis is a Clostridium difficile infection. In some embodiments, the dysbiosis is food poisoning. In some embodiments, the dysbiosis is chemotherapy-related dysbiosis.

In some embodiments, at least one bacterial strain and/or species is disclosed in ‘Stool substitute transplant therapy for the eradication of Clostridium difficile infection: ‘RePOOPulating the gut’, by Petrof et al. (2013), which is incorporated herein by reference in its entirety.

In some embodiments, at least one bacterial species is disclosed in Kurokawa et al., “Comparative metagenomics revealed commonly enriched gene sets in human gut microbiomes”, (2007) DNA Research 14: 169-181, which is incorporated herein by reference in its entirety.

In some embodiments, the at least one bacterial species is disclosed in U.S. Patent Application Publication No. 20150044173. Alternatively, in some embodiments, the at least one bacterial species is disclosed in U.S. Patent Application No. 20140363397. Alternatively, in some embodiments, the at least one bacterial species is disclosed in U.S. Patent Application No. 20140086877. Alternatively, in some embodiments, the at least one bacterial species is disclosed in U.S. Pat. No. 8,906,668.

In some embodiments, the method of the present invention can include evaluating at least one bacteria according to the disclosed methods in Takagi et al. (2016) “A single-batch fermentation system to simulate human colonic microbiota for high-throughput evaluation of prebiotics” PLoS ONE 11(8): e0160533.

In some embodiments, the at least one bacterial species is derived from a healthy patient. In some embodiments, the at least one bacterial species is derived from a healthy patient according to the methods disclosed in U.S. Patent Application Publication No. 20140342438.

In some embodiments, the at least one bacterial species and/or strain is derived from a patient by a method comprising:
a. obtaining a freshly voided stool sample, and placing the sample in an anaerobic chamber (in an atmosphere of 90% N2, 5% CO2, and 5% H2);
b. generating a fecal slurry by macerating the stool sample in a buffer; and
c. removing food particles by centrifugation, and retaining the supernatant.

In some embodiments, the supernatant is used to seed a chemostat according to the methods of U.S. Publication Number 20140342438.

Culture Methods According to some Embodiments of the Present Invention

The effectiveness of the method to determine a first metabolic profile of a subject having a dysbiosis can be limited by factors such as, for example, the sensitivity of the method (i.e., the method is only capable of detecting a particular bacterial strain if the strain is present above a threshold level.)

The effectiveness of the method to determine a second metabolic profile which treats a subject can be limited by factors such as, for example, the sensitivity of the method (i.e., the method is only capable of detecting a particular bacterial strain if the strain is present above a threshold level.)

In some embodiments, the threshold level is dependent on the sensitivity of the detection method. Thus, in some embodiments, depending on the sensitivity of the detection method, a greater amount of the at least one bacterial species is required to determine if there has been sufficient colonization of the subject.

In some embodiments, the at least one bacterial species and/or strain is cultured in a chemostat vessel. In some embodiments, the at least one bacterial strain selected from the group consisting of: Acidaminococcus intestinalis 14LG, Bacteriodes ovatus 5MM, Bifidobacterium adolescentis 20MRS, Bifidobacterium longum, Blautia sp. 27FM, Clostridium sp. 21FAA, Collinsella aerofaciens, Escherichia coli 3FM4i, Eubacterium desmolans 48FAA, Eubacterium eligens F1FAA, Eubacterium limosum 13LG, Faecalibacterum prausnitzii 40FAA, Lachnospira pectinoshiza 34FAA, Lactobacillus casei 25MRS, Parabacteroides distasonis 5FM, Roseburia faecalis 39FAA, Roseburia intestinalis 31FAA, Ruminococcus sp. 11FM, Ruminococcus species, Ruminococcus torques 30FAA; and any combination thereof, is cultured in a chemostat vessel.

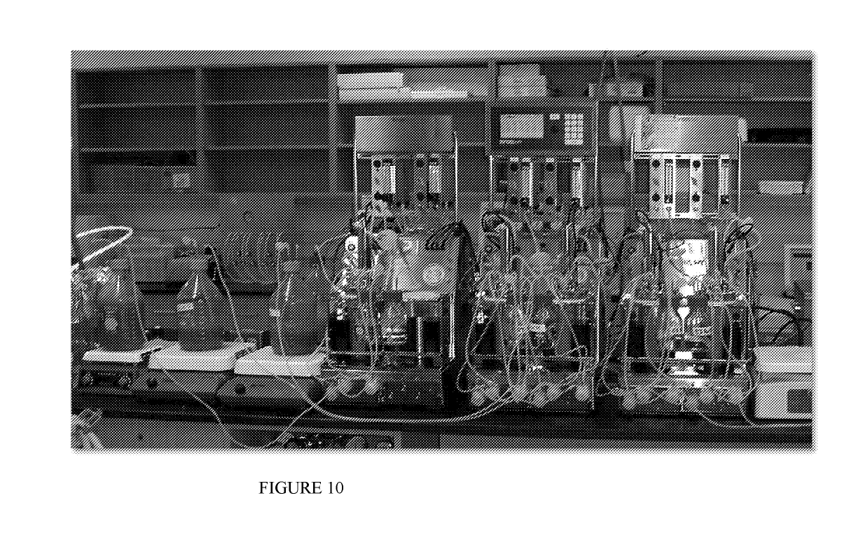

In some embodiments, the at least one bacterial species selected from the group consisting of: 16-6-I 21 FAA 92% Clostridium cocleatum; 16-6-I 2 MRS 95% Blautia luti; 16-6-I 34 FAA 95% Lachnospira pectinoschiza; 32-6-I 30 D6 FAA 96% Clostridium glycyrrhizinilyticum; 32-6-I 28 D6 FAA 94% Clostridium lactatifermentans; and any combination thereof, is cultured in a chemostat vessel. In some embodiments, the chemostat vessel is the vessel disclosed in U.S. Patent Application Publication No. 20140342438. In an embodiment, the chemostat vessel is the vessel described in FIG. 10.

In some embodiments, the chemostat vessel was converted from a fermentation system to a chemostat by blocking off the condenser and bubbling nitrogen gas through the culture. In some embodiments, the pressure forces the waste out of a metal tube (formerly a sampling tube) at a set height and allows for the maintenance of given working volume of the chemostat culture.

In some embodiments, the chemostat vessel is kept anaerobic by bubbling filtered nitrogen gas through the chemostat vessel. In some embodiments, temperature and pressure are automatically controlled and maintained.

In some embodiments, the culture pH of the chemostat culture is maintained using 5% (v/v) HCl (Sigma) and 5% (w/v) NaOH (Sigma). In some embodiments, the pH is between 6.8 to 7. In some embodiments, the pH is between 6.9 to 7. In some embodiments, the pH is between 6.8 to 6.9.

In some embodiments, the culture medium of the chemostat vessel is continually replaced. In some embodiments, the replacement occurs over a period of time equal to the retention time of the distal gut. Consequently, in some embodiments, the culture medium is continuously fed into the chemostat vessel at a rate of 400 mL/day (16.7 mL/hour) to give a retention time of 24 hours, a value set to mimic the retention time of the distal gut. An alternate retention time can be 65 hours (approximately 148 mL/day, 6.2 mL/hour). In some embodiments, the retention time can be as short as 12 hours.

In some embodiments, the culture medium is a culture medium disclosed in U.S. Patent Application Publication No. 20140342438.

### Materials and Methods

Preparation of Resistant Starch-Containing Substrates

Cornmeal from six maize lines (Table 2.1) was subjected to in vitro digestion and fermentation. Dried maize kernels were obtained from Dr. Michael Emes (University of Guelph, Ontario) and ground to allow passage through a 1mm screen using a cyclone mill (UDY Cyclone Sample Mill), giving a fine cornmeal. 30 g of the cornmeal was suspended in 500 ml of phosphate buffer (20 mM, pH 6.9, Na2HPO4 1.42 gL-1, KH2PO4 1.36 gL-1, NaCl 0.58 gL-1), autoclaved for 30 minutes at 121° C. and cooled with stirring on a magnetic stirrer to 37° C., immediately prior to the digestion procedure. The digestion was conducted under sterile conditions. Briefly, the solution was incubated at 37° C. on a magnetic stirrer and digestive enzymes (all sourced from Sigma Aldrich, Oakville, Ontario) were added in a stepwise manner: 1) pH was adjusted to 6.9±0.1 with the addition of NaOH (20% (w/v)), 0.5 mL human salivary α-amylase solution (10 mgmL-1 in CaCl2 1 mM) was added and incubated for 15 minutes; 2) pH was adjusted to 2.0±0.1 with the addition of HCl (20% (v/v)) and 1.25 mL porcine pepsin suspension (1 mgmL-1 in NaCl 9 gL-1) and incubated for 30 minutes; 3) pH was adjusted to 6.9±0.1with the addition of base (20% (w/v) NaOH), 10 mL of pancreatin (0.5 mgmL-1 in CaCl2 25 mM) and 12 g of bovine bile (Sigma Aldrich) were added and incubated for 3 hours. The digestion products were removed by dialysis, using sterile dialysis tubing with a molecular cut-off of 12-14 kDa (Servapor 44146, Serva Feinbiochemica GmbH & Co., Heidelberg, Germany) under constant stirring in ddH2O at 4° C. over 24hours. The retentate was transferred to sterile 50 mL conicals and freeze dried for 4 days (FreeZone®12 Liter Freeze Dry System, Labconco, Mo., USA). Using aseptic technique, dried substrates were ground using a mortar and pestle and passed through a 500 μm sieve to achieve a uniform particle size. Prepared starch substrates were stored at −20° C. until used for in vitro small scale batch fermentations.

TABLE 2.1
Maize mutants selected for analysis during in vitro studies

| Genotype | Mutation/Effect on starch |
|---|---|
| Cg102 | N/A (wild type) |
| Cg102wx | Lacks (GBSS), does not contain amylose |
| Cg102ae1-ref | Lacks (SBEIIb), amylopectin has longer chains |
| | and reduced branching |
| Cg102ae1-Elmore | inactive (SBEIIb), amylopectin has longer chains |
| | and reduced branching |
| Cgx333 | N/A (wild type) |
| Cgx333Su2 | Lacks (SSIIa), reduced amylopectin synthesis |

Resistant Starch Determinations:

Quantities of resistant starch (RS), soluble starch (SS), and total starch (TS) in the predigested and untreated cornmeal of the 6 maize lines were determined using the Megazyme RS assay kit (Megazyme International, Ireland). Briefly, the samples were digested at 37° C. with continuous shaking for 16 hours in the presence of 4 ml of pancreatic α-amylase (10 mgmL-1) containing amyloglucosidase (AMG) (3 UmL-1). Solubilised starch was separated from the undigested resistant starch by centrifugation at 3000 g and repeated washes with 50% ethanol. The RS pellet was dissolved by adjusting the pH through the addition of KOH (2M) with constant stirring. Both the SS and RS fractions were treated with AMG 10 μL (300 UmL-1) 20 minutes and 0.1 mL (3300 UmL-1) 30 minutes respectively at 50° C. Finally, 0.1 mL aliquots of these solutions were combined with 3 mL of glucose oxidase-peroxidase reagent (GOPOD) and incubated at 50° C. for 20 minutes before absorbance was measured at 510 nm against a reagent blank. RS and SS were calculated according to manufacturer instructions; the sum of these fractions was equal to the TS.

Chemostat Operation

Preparation of Single-Stage Chemostats

An Infors Multifors bioreactor system (Infors, Switzerland) was converted to chemostat operation by closing the condenser vent and bubbling nitrogen gas through the culture, creating an anaerobic environment and a positive pressure within the vessel that maintained the vessel contents at a constant volume of 400 mL. Temperature and pH were constantly monitored during the course of an experiment, maintaining a consistent 37° C. and a pH of 6.9-7.0 by the addition of acid (5% (v/v) HCl) and base (5% (w/v) NaOH). Vessels were constantly stirred and provided with a constant flow of fresh medium detailed in Table 2.2 at a rate of 400 mL/day resulting in a retention time of 24 hours to mimic the human distal colon. Prior to inoculation, each vessel was sampled aseptically and plated on fastidious anaerobe agar (Acumedia; Lansing, Mich.) supplemented with 5% defibrinated sheep blood (Hemostat Laboratories; Dixon, Calif.)(FAAb) and incubated both aerobically and anaerobically at 37° C. to ensure vessels were free of contamination.

The medium used was based on previous studies using a chemostat to mimic the human gut. Medium was prepared in 4 separate stock preparations (Table 2.2) and aseptically combined in a biological safety cabinet, as well 2.5 mL of antifoam B silicone emulsion (J.T. Baker; Center Valley, Pa.) was added to each liter of prepared. Preparations 1 and 4 were autoclaved while preparations 2 and 3 were filtered through 0.22 μm filters prior to addition. Media was checked for sterility by plating on FAAb and stored for less than 2 weeks at 4° C. before use.

Collection and Preparation of Fecal Inocula

The Research Ethics Board of the University of Guelph approved this study (REB#09AP011). Three healthy donors provided fresh fecal samples for this work: donor 2 (female, 42 years old), donor 5 (male, 44 years-old), and donor 9 (male, 25 years old). None of the donors had a history of chronic disease or a treatment of antibiotics within the year previous to the collection of samples for this study.

Fecal collection and preparation were conducted. Briefly, samples were collected in a sterile, lidded container in a nearby washroom and transferred within 5-10 minutes of voiding to an anaerobic chamber (atmosphere of 90% N2, 5% CO2 and 5% H2). Feces (5 g) were homogenized in 50 mL of degassed chemostat media for 1 minute using a stomacher (Tekmar Stomacher Lab Blender, Seward; Worthing, West Sussex, UK) producing a 10% (w/v) fecal slurry. Large particles were removed with low speed centrifugation for 10 minutes and 175 xg. The supernatant functioned as the fecal inoculum for the chemostats in this study.

It is common practice to remove large food particles by gentle centrifugation of stool prior to chemostat inoculation. Previous studies have shown that low-speed centrifugation did not introduce significant bias to the microbial community of the supernatant compared to that of the original fecal sample.

TABLE 2.2
Growth medium composition (per litre of prepared media) used in the
single-stage chemostat model.

| Preparation | Reagent | Weight (g) |
|---|---|---|
| 1 | Peptone water^{b} | 2 |
| (800 mL H₂O) | Yeast extract^{c} | 2 |
| | NaHCO₃^{b} | 2 |
| | CaCl₂^{a} | 0.01 |
| | Pectin (from citrus)^{a} | 2 |
| | Xylan (from beechwood)^{a} | 2 |
| | Arabinogalactan^{a} | 2 |
| | Starch (from wheat, unmodified)^{a} | 5 |
| | Casein^{d} | 3 |
| | Inulin (from Dahlia tubers)^{d} | 1 |
| | NaCl^{a} | 0.1 |
| 2 | K₂HPO₄^{a} | 0.04 |
| (50 mL H₂O) | KH₂PO₄ | 0.04 |
| | MgSO₄^{e} | 0.01 |
| | Hemin^{a} | 0.005 |
| | Menadione^{a} | 0.001 |
| 3 (50 mL | Bile salts^{a} | 0.5 |
| H₂O) | L-cysteine HCl^{b} | 0.5 |
| 4 | Porcine gastric mucin (type II) | 4 |
| (100 mL H₂O) | | |

Superscripts signify chemical suppliers:
^{a}Sigma-Aldrich (Oakville, Ontario);
^{b}Thermo Fisher Scientific (Ottawa, Ontario);
^{c}BD (Franklin Lakes, New Jersey);
^{d}Alfa Aesar (Ward Hill, Massachusetts);
^{e}BDH (Radnor, Pennsylvania).

Inoculation, Operation and Sampling

Chemostat vessels were inoculated with the addition of 100 mL of 10% fecal inoculum (Section 2.3.2) to 300 mL of sterile chemostat medium. Stirring and pH control were turned on immediately following inoculation and remained on for the duration of the run. The culture was allowed to establish itself for 24 hours within the vessel before starting the feed pump. Daily sampling of the chemostat vessel included the addition of 20 drops of antifoam B silicone emulsion (J.T. Baker; Center Valley, Pa.) and the removal of 4 mL of culture through the vessels sampling port. Samples were aliquoted into 2×2 mL tubes and archived at −80° C. for subsequent DNA extraction.

In Vitro Static Batch Starch Fermentations

Chemostats seeded with feces from donors 2, 5, and 9 were operated as previously described (section 2.3.3) and stable, steady-state communities from these chemostats were used as the inoculum source for all for all subsequent fermentations. Static batch fermentations with a 50 mL working volume were conducted in a 37° C. anaerobic incubator following the procedure from a previous study. Briefly, 0.5 g of each starch substrate was aseptically transferred to 100 mL bottles in a biological safety cabinet to achieve a final concentration of 10 gL-1.

Immediately prior to the addition of the chemostat culture, 45 mL of sterile, pre-reduced basal culture media (pH 6.9 ±0.1) (Table 2.3) was added to the bottles. Each bottle was inoculated with 5 mL of fresh chemostat culture for a final concentration of 100 mL/L. Control fermentations contained either 50 mL of fermentation buffer and 0.5 g of each starch (10 gL-1) or 45 mL of fermentation buffer with 5 mL of fresh chemostat culture (100 mL/L).

The static batch fermentations were run for 48 hours, without pH control, at 37° C., within an anaerobic incubator. Duplicate 2 mL samples were taken at 0, 4, 8, 24, and 48 hours post inoculation and frozen at −80° C. for DNA and VOC analysis. Multiple starch substrate fermentations were conducted during these experiments, and each fermentation was labelled according to starch substrate, biological replicate #, technical replicate # (roman numerals), and sample time point. For example (Cg102 1i-48 h) represents a sample taken 48 h post inoculation from the first biological and first technical replicate of a fermentation containing the starch substrate Cg102.

Chemostat Feeding Trial

Standard chemostat media was supplemented with predigested Hi-maize 260 (high RS) (National Starch and Chemical, Manchester, United Kingdom) or corn starch (control) (Sigma-Aldrich, Oakville, Ontario) to mimic the quantities consumed during in vivo feeding trials, total 30 g/day prior to digestion. 120 g of Hi-maize 260 and cornstarch were digested in 30 g batches according to the method described previously (Section 2.1) with slight modifications. The starches were boiled for 20 minutes in phosphate buffer (20 mM, pH 6.9, Na2HPO4 1.42 gL-1, KH2PO4 1.36 gL-1, NaCl 0.58 gL-1), and after dialysis the retenate was directly added to the chemostat media (solution 1, Table 2.1) without freeze drying prior to solution 1 being autoclaved, the remainder of the media was prepared as previously described.

Two pairs of ‘twin’ chemostat vessels (two identical chemostat vessels inoculated with the same inocula) (D5-1 and D5-2) and (D9-R1 and D9-R2) were operated without any experimental manipulation until the vessels reached steady state. After establishment of steady state the media was changed on vessels D5-1(RS+) and D9-R1(RS+) to provide the equivalent of 30 g/day (prior to digestion) of Hi-maize 260 for duration of 4 days. Media was similarly changed on vessels D5-2(CS+) and D9-R2(CS+) providing an equal volume 30 g/day (prior to digestion) of corn starch for 4 days (control vessels). After the 4 days of increased starch all vessels were returned to the basal chemostat media and run for 4 days to provide a washout period before terminating the experiment. Vessels were sampled as previously described throughout the course of the experiment.

TABLE 2.3
Basal culture medium (per litre of prepared media) used in small scale
batch fermentations.
Reagent (per liter)

| | | | |
|---|---|---|---|
| Peptone waterb | 2 g | NaHCO3b | 2 g |
| Yeast extractc | 2 g | Hemind | 0.005 g |
| NaCla | 0.1 g | L-cysteine HClb | 0.5 g |
| K2HPO4a | 0.04 g | Bile saltsa | 0.5 g |
| KH2PO4b | 0.04 g | Tween 80b | 2 mL |
| MgSO4e | 0.01 g | Vitamin K1a | 10 μL |
| CaCl2a | 0.01 g | | |

Superscripts signify chemical suppliers:
aSigma-Aldrich (Oakville, Ontario);
bThermo Fisher Scientific (Ottawa, Ontario);
cBD (Franklin Lakes, New Jersey);
dBDH (Radnor, Pennsylvania).

DNA Extraction

DNA was extracted from archived samples following a modified protocol, utilizing a combination of bead-beating and components of two commercially available kits. Briefly, 200 mg of glass beads, 300 μL of SLX buffer (Omega Bio-Tek E.Z.N.A.® Stool DNA kit; Norcross, Ga.), and 10 μL of proteinase K (20 mgmL-1, in 0.1 mM CaCl2) were added to 2 mL screw cap tubes. Each sample was thawed and thoroughly mixed before 200 μL was aliquoted into screw-capped Eppendorf tubes which were placed into a bead beater and processed for 4 minutes at 3000 rpm using Disruptor Genie (Scientific Industries, Bohemia, N.Y.). Samples were incubated for 10 minutes at 70° C., 5 minutes at 95° C., then 2 minutes on ice. 100 μL of Buffer P2 (Omega Bio-Tek E.Z.N.A.® Stool DNA kit; Norcross, Ga.) was added to the tubes and vortexed for 30 seconds followed by an additional incubation for 5 minutes on ice. Samples were centrifuged at 20450×g for 5 minutes and the supernatants transferred to new 1.5 mL tubes with 200 μL of HTR reagent (Omega Bio-Tek E.Z.N.A.® Stool DNA kit; Norcross, Ga.), vortexed for 10 seconds and incubated at room temperature for 2 minutes. Finally samples were centrifuged at 20450×g for 2 minutes, supernatants were transferred into Maxwell®16 DNA Purification Kit cartridges (Promega; Madison, Wis.) with the remainder of the extraction protocol completed according to the Maxwell kit instructions.

Live/Dead Cell Comparison

Select samples were tested to determine if changes in community dynamics were influenced by the presence of DNA originating from dead cell populations, using the PhAST Blue photo-activation system (PhAST Blue, GenIUL, Barcelona, Spain). DNA from dead (permeable) cells was deactivated using ethidium monoazide (EMA) following the manufacturer's instructions. Immediately following sampling at 0 and 48 hours, samples were homogenized using a micro pipette, and a 200 μL aliquot was added to a provided reagent tube and gently vortexed. Samples were incubated in an ice water bath with intermittent mixing on a vortex mixer for 10 minutes and transferred to a sterile reaction tube. Samples were photo-activated with the provided PhAST Blue system apparatus (a blue light generator) using the preset manufacturer settings (15 minutes at 100% intensity). Samples were spun at 20450×g for 5 minutes, the supernatant was discarded and the pellet resuspended in 200 μL sterile phosphate buffered saline (PBS), before DNA extraction following the previously described protocol.

Denaturing Gradient Gel Electrophoresis (DGGE) Analysis

DNA was extracted from all batch fermentations at 0 and 48 hours post inoculation and from chemostat vessels every 2 days, for DGGE analysis determining similarity and changes in community dynamics. PCR, PCR purification and concentration, DGGE, and gel analysis were conducted. Primers HDA1 and HDA2-GC were used to amplify the V3 region of the 16S rRNA gene (339-539bp, Escherichia coli numbering). The cycling conditions were: at 92° C. for 2 min, (92° C. for 1 min, 55° C. for 30 sec, 72° C. for 1 min)×35 cycles; 72° C. for 10 min. Extracted DNA severed as a template with each sample being amplified in three identical PCR reactions. PCR products were analysed by agarose gel electrophoresis (2% agarose w/v, in 1×Tris-acetate-EDTA (TAE), 40 minutes at 80V) to ensure PCR reactions were successful.

Identical samples were pooled and concentrated with an EZ-10 Spin Column PCR Products Purification Kit (Biobasic; Markham, Ontario) following a slightly modified protocol. Samples were eluted in 40 μL HPLC grade water and mixed with 10 μL DGGE loading dye (0.05% (v/v) bromophenol blue; 0.05% (v/v) xylene cyanol; 70% (v/v) glycerol in HPLC grade water; Bio-Rad DCode Manual). A DGGE standard ladder developed using previously extracted DNA from human gut bacterial isolates available in house was run on the outer and middle lanes of all DGGE gels. DNA samples included within the ladder were from the following bacterial isolates: Coprobacillus sp. (1/2/53), Enterococcaceae sp. (30/1 aka HMP#323), Veillonella sp. (5/2/43 FAA), Clostridium sp. (1/1/41 Al FAA CT2, aka HMP#174), and Propionibacterium sp. (7/6/55B FAA). The ladder was prepared by amplifying the DNA of each strain using HDA1 and HDA2-GC primers as previously described, and pooling the products in equal ratios.

The DCode System (Bio-Rad Laboratories, Hercules, Calif.) was used to perform DGGE with a 6% (v/v) polyacrylamide gel, following a previously described protocol A 30-55% denaturing gradient consisting of urea and formamide was utilized to separate the purified PCR products. Gels underwent electrophoresis at 60° C. in 1×TAE buffer for 5 hours at 120V. Gels were stained then destained in ethidium bromide (100 μl in 1 L 1×TAE) (Sigma-Aldrich, Oakville, Ontario) and ddH2O respectively for 10 minutes each. SynGene G-Box gel documentation system running GeneSnap software (version 6.08.04, Synoptics Ltd; Cambridge, UK) was utilized to capture images of the gels. During capture the automatic exposure tool of the GeneSnap software was used to normalize images for saturation.

Syngene GeneTools software (version 4.01.03, Synoptics Ltd) was used to analyze DGGE gels. DGGE banding patterns between sample profiles were analyzed for similarities through the calculation of Pearson correlation coefficient values (similarity index (SI) values) generating a similarity matrix. SI values ranged from 0 to 1; a value of 1 indicated that the two profiles contained identical banding patterns, while a value of 0 indicated no bands in common between the two profiles. SI values were multiplied by 100 to obtain Correlation coefficients (% similarity index (% SI) values). % SI values were utilized to generate a dendrogram using the unweighted pair group with mathematical averages (UPGMA) method.

Identical ladder samples within the same DGGE gel were used to calculate gel-specific “cut-off thresholds” by comparing the similarity of the banding profiles. In theory, identical ladder samples should be 100% similar; however the % SI values are always less than 100% due to the experimental error associated with DGGE and the variation of the denaturing gradient throughout the gel. The % SI value between ladder samples defined the “cut-off threshold”, therefore samples with % SI values greater than that of the “cut-off threshold” were considered identical, while % SI values within 5% of the “cut-off threshold” were considered similar.

Community Dynamics and Between Vessel Similarity

Community dynamics were illustrated with moving window correlation analysis, assessing changes within a community over a period of time. These plots were used to confirm that communities reached a steady state and to assess the community stability in response to the in vitro feeding trials. Each point on the plot represents the percent change (100-% SI) between DGGE profiles of samples originating from the same vessel on days (x−2) vs. (x). Changes in community similarity between ‘twin vessels’ were determined using % SI values. Each point on the plot represents the % SI between the two vessels on identical days of treatment course. Decreases in similarity represent divergence in community structures between the vessels in response to the different treatments.

DGGE Pattern Analysis: Non-Metric Multidimensional Scaling

Non-metric multidimensional scaling (NMDS) is an ordination technique that aims to graphically summarize complex relationships within datasets. NMDS does not require linear relationships between variables, and attempts to preserve the ranked order of similarity between samples. Therefore, samples with a more similar community composition are positioned more closely to one another. DGGE profiles were analyzed for similarities through the calculation of Pearson correlation coefficient values, generating similarity matrixes used to create NMDS plots for each DGGE gel using XLstat (Addinsoft, hhttp://www.xlstat.com). Kruskal's stress formula 1 was used to determine the degree to which the plot accurately represents the similarity matrix, stress values <0.1 represent good ordinations with a low risk of drawing false conclusions about the patterns. Values 0.1<x<0.2 still produce a useable model although values close to 0.2 can be misleading and caution should be taken when drawing conclusions on the results. All NMDS plots are presented as two-dimensional models of the relationships between the DGGE profiles of the small scale batch fermentations.

SPME-GC-MS Parameters

Volatile organic compounds (VOCs) were extracted and analysed from small scale batch fermentations at 0 and 48 hours post inoculation using solid phase microextraction (SPME) coupled with gas chromatography-mass spectrometry (GC-MS). Changes in metabolites present after the fermentation of the starch substrates were determined following a slightly modified protocol for the analysis of fecal VOC. Briefly, samples archived at −80° C. were thawed at room temperature mixed thoroughly before 1 mL was transferred to a 10 mL glass vial and capped with a crimp top lid containing a PTFE silicone septum (MicroLiter Analytical Supplies, Inc., Ga., USA).

The SPME-GC-MS method was carried out using a Bruker Scion 436GC instrument equipped with automated SPME autosampler. The analytical column was a ZB-624 (Phenomenex, Torrance, Calif., USA) capillary column (30 m ×0.25 mm; film thickness 1.40 mm). The injector port was set at 280° C. The oven temperature conditions were as follows: starting at initial temperature of 35° C. for 5 min, the temperature was increased to 250° C. at 7° C. min-1 rate and held for 12 min giving a total run time of 47.71 min. The flow of the carrier gas (helium, purity>99.999%) was maintained at 1.0 mLmin-1 in constant flow mode. The GC-MS was programmed to perform a split injection, with samples injected using a 1:20 split ratio. The SPME injector parameters were as follows: agitator temperature 75° C., sample preincubation time 15 min., incubation time with fiber (extraction time) 30min., desorbtion time 5 min. An SPME fiber assembly made of Divinylbenzene/Carboxen/Polydimethylsiloxane (DVB/CAR/PDMS) (Supelco, Bellefonte, Pa., USA) was used.

The mass spectrometer (Scion TQ) was equipped with an electron impact (EI) ion source. All experiments were carried in the positive ion mode. The source temperature was set at 200° C., and the energy was 70 eV. The multiplier voltage was set to 900 V. Data was acquired in full scan mode from 30-300 m/z at the rate of 4 scan/min with a 4 min delay. An empty glass vial was prepared as a control and stored under identical conditions as that of the sample vials and analyzed following the same protocol. Tentative identification of peaks of interest were performed by comparison to the NIST mass spectral database (National Institute of Standards and Technology, Gathersburg, Md.).

GC-MS Data Processing and Statistical Analysis

Raw GC/MS data was converted into .xml format using mzXML Conversion Utility (Bruker) and subsequently processed using the XCMS software package (version 1.36.1) implemented in the R language (version 2.15.3, R-Foundation for statistical computing, www.Rproject.org) for automatic peak detection and peak alignment using previously described parameter. The resulting tab delimited table output from R was imported into Microsoft Excel software (Microsoft, Redmond, Wash.), ion features were normalized to total peak area and arranged in a table containing mass spectral features as m/z retention time pairs, sample names, and peak areas and subsequently imported into SIMCA-P+13.03 (Umetrics, Umea, Sweden) for statistical analysis using PCA and orthogonal projections to latent structures-discriminant analysis (OPLS-DA). Variables were mean-centered and pareto-scaled for PCA and OPLS-DA, PCA score plots were analyzed to determine the general structure of the data sets from the fermentations using fecal inoculum from the 3 donors. OPLS-DA was used to distinguish differences in metabolite profiles between two classes (0 h and 48 h sampling time points); models were cross-validated 7 times with 1/7th of the data left out randomly for each round of validation and the reliability of the models was assessed using analysis of variance of cross-validated residuals (CV-ANOVA). Key metabolites for the separation of the two time points were identified using variable influence on projection (VIP) values from the OPLS-DA model that were above a statistically significant threshold (VIP values>1). Metabolites meeting the VIP cut-off were individually assessed for differences between the two groups in GraphPad Prism (Version 5, GraphPad software Inc, Calif., USA) using the Mann-Whitney-Wilcoxon test, samples were considered significantly different if the P-value was <0.05.

Metabolic differences between the fermentation of the 6 starch substrates for each donor's fecal microbiota were determined by comparing the 48 h fermentation samples to one another. PCA and OPLS-DA models were generated as described above, PCA score plots were analyzed to determine the general structure of the data sets. Samples were placed into 6 defined classes based on starch substrate, and compared as pairs with one another using OPLS-DA modeling as previously described.

Genome sequences

The data for this study includes the draft genome sequences (in contig form) of thirty-three bacteria strains, which are disclosed in Table 4. The bacterial genomes were sequenced using the Illumina MiSeq Platform. Species were named according to closest match by comparison of full-length 16S rRNA genes and may not reflect the true speciation of the bacteria, for simplicity bacteria used in Part I have been given a separate identity as strain A or strain B, Table 1 provides the true identification for these strains.

Study Design

The study includes three stages. The first stage focused on comparing the genomes of species for which pairs of strains had been included in the RePOOPulate study (Petrof et al.) (also referred to as the “original RePOOPulate protoype” or “original RePOOPulate ecosystem”). The genomes of six pairs of species strains that matched closely by full-length 16S sequence alignment were compared in order to search for redundancies. Multiple strains of these bacteria were originally chosen for inclusion in the RePOOPulate ecosystem based on morphological and behavioral differences in the cultured bacteria. The goal of this portion of the project was to determine whether the use of multiple strains was redundant or if there is a true genetic difference that validates a biologically necessity to include both strains for the maintenance of ecological balance.

The second stage of the project focused on developing a broad pipeline for determining the genetic coverage of the KEGG pathways. KEGG, which stands for Kyoto Encyclopedia of Genes and Genomes, is a commonly used resource for pathway analysis and contains data associated with pathways, genes, genomes, chemical compounds and reaction information. Part II of the report will focus on comparing the KEGG pathways for the entire RePOOPulate ecosystem, in search of keystone bacterial species and pathways, as well as species that may be biochemically redundant.

The third stage of the project focused on determining whether the bacterial genes included in RePOOPulate provide adequate coverage of the necessary biochemical pathways without high levels of genetic redundancy. Part III of the report shows the entire RePOOPulate community's coverage of the KEGG pathways as compared to that of a “healthy” human microbiome. This allowed for an examination of the overall coverage of the KEGG pathways to determine how close the RePOOPulate community emulates the true microbiota of the human gut.

Part I: Redundancy within Strain Pairs

### Methods

### Mauve Alignment

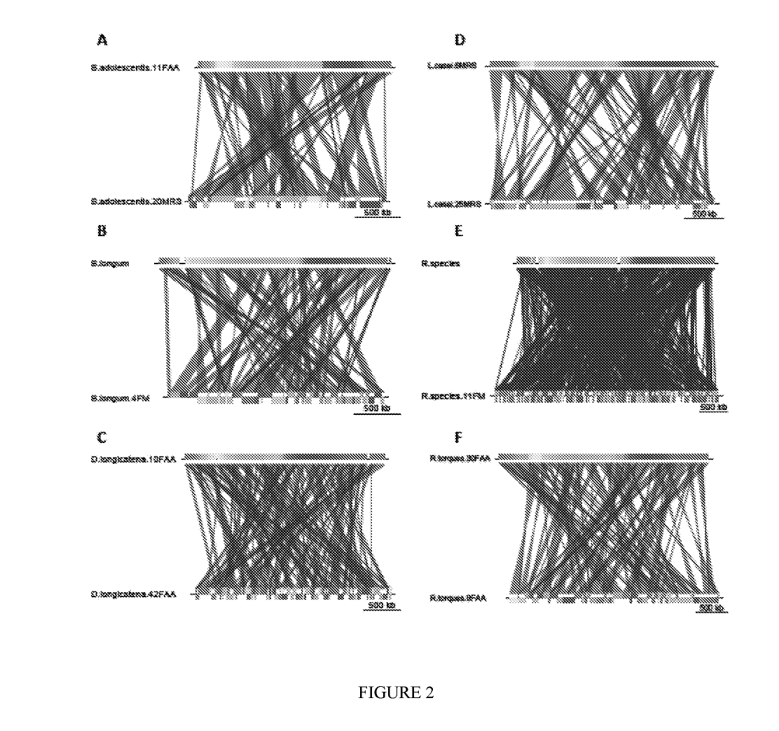

The original RePOOPulate prototype ecosystem included six species of bacteria with two separate strains, for a total of twelve bacterial strains. The whole genome data for both strains of these six species of bacteria were compared to test for redundancy. The pairs of genomes were aligned and compared using the progressive Mauve function of the genome alignment visualization tool Mauve. The resulting alignment backbone files were loaded into R and the package genoPlotR (pseudo-code provided) was used to create more dynamic images than those provided by Mauve (FIG. 2). Following alignment, strains for each species were assigned as either strain A or strain B to simplify further analysis of comparison results (Table 1).

FIG. 2 shows sequence alignment diagrams for mauve alignments, showing the alignment of the strain pairs for the six species analyzed in Part I and were created using Mauve and the R package genoPlotR. FIG. 2A shows Bifidobacterium adolescentis sequence comparison of strain A to strain B. FIG. 2B shows Bifidobacterium longum sequence comparison of strain A to strain B. FIG. 2C shows Dorea longlcatena sequence comparison of strain A to strain B. FIG. 2D shows Lactobacillus casei sequence comparison of strain A to strain B. FIG. 2E shows Ruminococcus torques sequence comparison of strain A to strain B. FIG. 2F shows Ruminococcus obeum sequence comparison of strain A to strain B.

Table 1 shows strain designation for part I, specifically determining redundancy within strain pairs. Identification of the strains referred to as strain A and strain B for each of the pairwise comparisons of the six species for which two strains were included in the original RePOOPulate ecosystem. Names in the table indicate the name given on the RAST server and bracketed numbers indicate the RAST genome ID number.

TABLE 1

| | Bifidobacterium | Bifidobacterium | Dorea | Lactobacillus | Ruminococcus | Ruminococcus |
|---|---|---|---|---|---|---|
| Strai [symbol] | [symbol] | [symbol] | [symbol] | [symbol] | [symbol] | [symbol] |
| A | Bifidobacterium | Bifidobacterium | Dorea | Lactobacillus | Ruminococcus | Ruminococcus |
| | adolescentis | longum | longicatena | casei | speci [symbol] | torques |
| B | Bifidobacterium | Bifidobacterium | Dorea | Lactobacillus | Ruminococcus | Ruminococcus |
| | adolescentis | longum | longicatena | casei | sp. 11FM | torques |

[symbol]

indicates data missing or illegible when filed

Comparison using SEED viewer

The draft genomes used in this analysis had been previously annotated and stored on the RAST server. RAST uses subsystem-based annotation, which identifies protein-encoding, rRNA and tRNA genes, assigns functions to the genes, predicts which subsystems are represented in the genome and uses this information to reconstruct the metabolic network. A subsystem is defined as a collection of functional roles, which together implement a specific biological process or structural complex. The subsystems-based approach is built upon the principle that the key to improved accuracy in high-throughput annotation technology is to have experts annotate single subsystems over the complete collection of genomes, rather than having an annotation expert attempt to annotate all of the genes in a single genome. The annotated genomes are maintained in the SEED environment, which supports comparative analysis. Following genome pair alignment and visualization, functional and sequence comparison of each strain pair was completed using the SEED Viewer accessed through the RAST server.

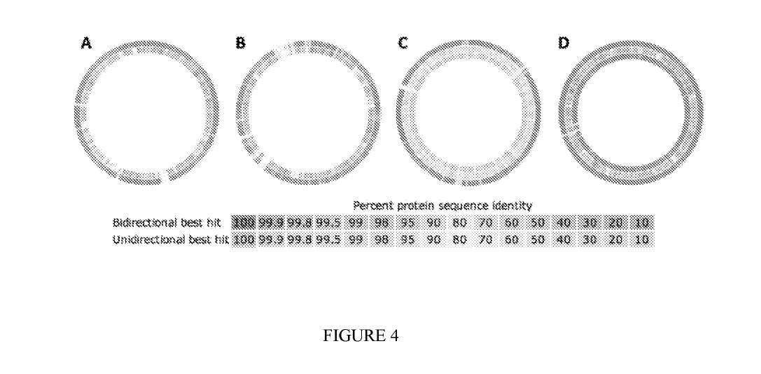

Functional comparison was used to identify subsystem-based differences using the annotated draft sequences. The functional comparison output provided consists of a table of identified subsystems indicating which subsystems were shared and which were unique to only one strain. The results of each of the six comparisons were exported in tab-separated value tables and examined in Microsoft Excel. A sequence comparison was then completed using the SEED Viewer to examine protein sequence identity and determine average genetic similarity. The image outputs were downloaded in graphics interchange format (gif) and textual results of this comparison were exported as tab-separated value tables and examined in Microsoft Excel. Protein sequence identity was examined both with and without the inclusion of hypothetical protein data. Sequence comparison was completed using both strain A as a reference and strain B as a reference since results differed slightly when different strains were used. When possible, strains were also compared to nearest available taxonomic neighbor in order to compare protein sequence similarity to that found in other bacterial strains within the same genus or species (FIG. 4). Data suggested that the genome size and the number of contigs could be confounding factors in the results for sequence comparison. This was examined using linear modeling in R. The data in Table 6 was saved as a comma-separated value file and loaded into R. Two linear models were fitted to compare the average percent protein sequence identity to genome size and to number of contigs (pseudo-code provided).

FIG. 4 shows SEED viewer sequence comparison figures for the closest available species match. FIG. 4A shows a comparison of reference Bifidobacterium adolescentis strain A to strain B (outer ring) and Bifidobacterium adolescentis (1680.3) (inner circle). FIG. 4B shows the sequence comparison of Bifidobacterium longum strain A to strain B (outer ring) and Bifidobacterium longum DjO10A (inner ring). FIG. 4C shows the sequence comparison of Dorea longicatena strain A to strain B (outer ring) and Dorea formicigenerans ATCC27755 (middle ring) and Dorea longicatena DSM 13814 (inner ring). FIG. 4D shows sequence comparison of Lactobacillus casei strain B to Lactobacillus casei strain A (outer ring) and Lactobacillus casei ATCC 334 (middle ring) and Lactobacillus casei BL23 (inner ring). No Ruminococcus species were openly available for comparison purposes on the SEED viewer.

Table 6 shows summary statistics for strains analyzed in Part I, showing redundancy within strain pairs. Table 6 includes the size of the genome in number of base pairs, the number of contigs in the draft sequences used, the percent similarity to the closest match based on full-length 16S sequence alignment (inferred from original RePOOPulate paper), the total number of subsystems, coding sequences and RNAs identified using the SEED viewer, and the average percent protein sequence identity calculated in Microsoft Excel using data obtained from the Seed viewer (the listed strain is the reference strain for the comparison of strain pairs).

### KEGG Pathway Analysis

KAAS (KEGG Automatic Annotation Server) was used to provide functional annotation of the genes in the draft genomes (contigs) by BLAST comparison against a manually curated set of ortholog groups in the KEGG GENES database. The amino acid FASTA files for the twelve genomes examined in Part I were uploaded to KAAS and annotated using the prokaryotes gene data set and the bi-directional best hit assignment method, recommended for draft genome data. The result contains KEGG Orthology (KO) assignments and automatically generated KEGG pathways. The lists of KO assignments (KO IDs) were downloaded and compared in Microsoft Excel. Lists of KO IDs shared between pairs of strains and lists of KO IDs specific to one strain but not the other were created using Microsoft Excel spreadsheet tables. These lists were then used to create a final list of KO IDs with weights that matched the number of replicates of a KEGG orthology assignment and colors determined by whether or not an ID was shared (green for shared, red for strain A, blue for strain B). The final lists (one for each of the six species) were then imported into the program iPath2.0: interactive pathway explorer. iPath is a web-based tool for the visualization, analysis and customization of the various pathways maps. The current version provides three different global overview maps including: a map of metabolic pathways, constructed using 146 KEGG pathways, giving an overview of the complete metabolism in biological systems; a regulatory pathways map, which includes 22 KEGG regulatory pathways; and a biosynthesis of secondary metabolites map, which contains 58 KEGG pathways.

The lists of KO IDs created were matched to the internal list used by iPath2.0 before mapping; this removed several KO IDs since iPath2.0 does not include all available KO IDs in the mapping program. The matched lists were then used to create custom maps for each of the six strain comparisons. Lists of conflicts, in which KO IDs with different colors or weights fell within the same pathway, were automatically created through the mapping process for each strain comparison. The ipath2.0 program automatically resolves these conflicts by random choice. This method of resolution was not ideal for this study design; instead conflicts were resolved manually. Any color conflicts were resolved to be green, since a conflict in color meant the pathway was shared and therefore not unique. Any conflicts between weights were resolved by taking the average weight (rounded to the nearest whole number) or the least conflicting weight, in cases where a single KO ID conflicted with multiple KO IDs of the same weight. The final maps and lists of unique KO IDs were then analyzed to determine which pathways were unique to one strain and whether redundancies could be removed.

### Results

### Resistant Starch Determinations in Different Maize Lines

All starch substrates contained a considerable amount of total starch; however the total starch content decreased for all samples following in vitro human digestion. The total starch content of the undigested samples ranged from 61.38±0.54 to 74.21±2.88 g/100 g dry solids, while for digested samples this ranged from 52.89±2.08 to 66.20±0.08 g/100 g dry solids (Table 3.1). All samples contained less than 70% total starch indicating that, as expected, they were not pure starches (the samples were prepared from fine-ground, maize kernels which, as well as starch, contain protein, fat, and cellular material).

Cg102ae1-ref and Cg102ae1-Elmore contained the most RS prior to digestion (10.25±0.20 and 9.98±2.12 g/100 g dry solids respectively). Following in vitro digestion these same genotypes again contained the most RS (5.68±0.13 and 4.78±0.25 g/100 g dry solids respectively). In contrast, Cg102wx contained the lowest levels of RS both before and after in vitro digestion (0.13±0.003 and 0.03±0.0003 g/100 g dry solids respectively) (Table 3.1).

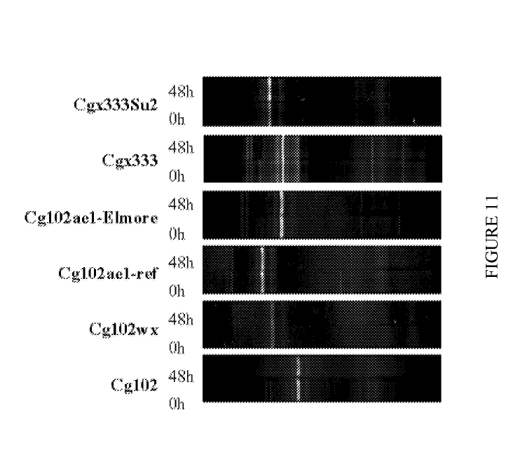

Although steps were taken to maintain sterility of the starch substrates during the in vitro digestions all preparations resulted in some level of contamination. FIG. 11 displays DGGE profiles of the six starch substrate controls after 0 h and 48 h in sterile anaerobic basal culture media. DGGE analysis resulted in a limited number of bands for each starch substrate. These bands did not appear prominently in the fermentations containing fecal inocula. Paired 0 h and 48 h samples were on average 97.7% similar, indicating that the contamination present did not contribute to changes observed during the small scale fermentations (Table 3.2).

FIG. 11 shows DGGE profiles comparing microbial contamination present in the six pre-digested starch substrate controls after 0 and 48 hours in sterile anaerobic basal culture media.

TABLE 3.1
Table 3.1 Resistant, soluble and total starch content of starch substrates pre- and post
in vitro human digestion, quantified using the Megazyme resistant starch assay kit
(Megazyme International, Ireland).

| | | | |
|---|---|---|---|
| | Resistant starch | Soluble starch | Total Starch |
| Starch sample | (g/100 g dry sample) | (g/100 g dry sample) | (g/100 g dry sample) |

Undigested

| | | | |
|---|---|---|---|
| Cg102 | 0.41 ± 0.15 | 65.10 ± 0.40 | 65.51 ± 0.55 |
| Cg102wx | 0.13 ± 0.00 | 64.13 ± 1.19 | 64.26 ± 1.20 |
| Cg102ae1-ref | 10.25 ± 0.20 | 51.13 ± 0.73 | 61.38 ± 0.53 |
| Cg102ae1-Elmore | 9.98 ± 2.11 | 52.49 ± 3.32 | 62.47 ± 1.20 |
| Cgx333 | 0.30 ± 0.05 | 73.90 ± 2.81 | 74.20 ± 2.87 |
| Cgx333Su2 | 1.63 ± 0.04 | 63.04 ± 0.47 | 64.67 ± 0.42 |

Digested

| | | | |
|---|---|---|---|
| Cg102 | 0.44 ± 0.00 | 55.95 ± 3.16 | 56.39 ± 3.15 |
| Cg102wx | 0.03 ± 0.00 | 55.75 ± 1.25 | 55.78 ± 1.25 |
| Cg102ae1-ref | 5.68 ± 0.13 | 47.20 ± 1.95 | 52.89 ± 2.07 |
| Cg102ae1-Elmore | 4.78 ± 0.25 | 51.01 ± 1.03 | 55.78 ± 1.28 |
| Cgx333 | 0.80 ± 0.01 | 65.39 ± 0.06 | 66.20 ± 0.08 |
| Cgx333Su2 | 2.73 ± 0.07 | 56.06 ± 0.45 | 58.79 ± 0.38 |

Values are means ± standard error,
n = 3

TABLE 3.2
Correlation coefficients (% SI) comparing microbial contamination present
in the six pre-digested starch substrates after 0 and 48 hours in sterile
anaerobic basal culture media.

| Starch Substrate | 0 h vs. 48 h |
|---|---|
| Control | % SI |
| Cg102 | 99.4 |
| Cg102wx | 93.9 |
| Cg102ae1-ref | 99.2 |
| Cg102ae1-Elmore | 98.4 |
| Cgx333 | 98.0 |
| Cgx333Su2 | 99.2 |

Small Scale Batch Fermentations—Rational

Small scale in vitro batch fermentations have been used for many years to study the effects that various substrates have on the gut microbiota. This technique requires collection of fresh feces from healthy donors on multiple occasions making studies difficult and time consuming. The use of a chemostat as a single-stage, distal model of the human gut is an effective means to culture fecal communities that are reproducible, stable and maintain a high level of diversity similar to that of the original fecal inocula. In this study we used a chemostat model to culture and maintain fecal communities from healthy donors such that they could be used repeatedly as a reservoir for in vitro small scale batch fermentations, thereby eliminating the need to sample fecal donors multiple times, and ensuring no batch-to-batch variability.

### Establishment of Steady State Communities in a Single Stage Distal Gut Model

Three separate chemostat runs were used as the fecal inocula source for in vitro small scale batch fermentations: 1) a single vessel inoculated with feces from ‘donor 2’, single donation (V2-1); 2) a single vessel inoculated with feces from ‘donor 5’, single donation (V5-1); 3) a single vessel inoculated with feces from donor 9, first donation (V9-1). Runs V5-1 and V9-1 were analyzed until day 40 and 41 respectively, while V2-1 was analyzed until day 38.

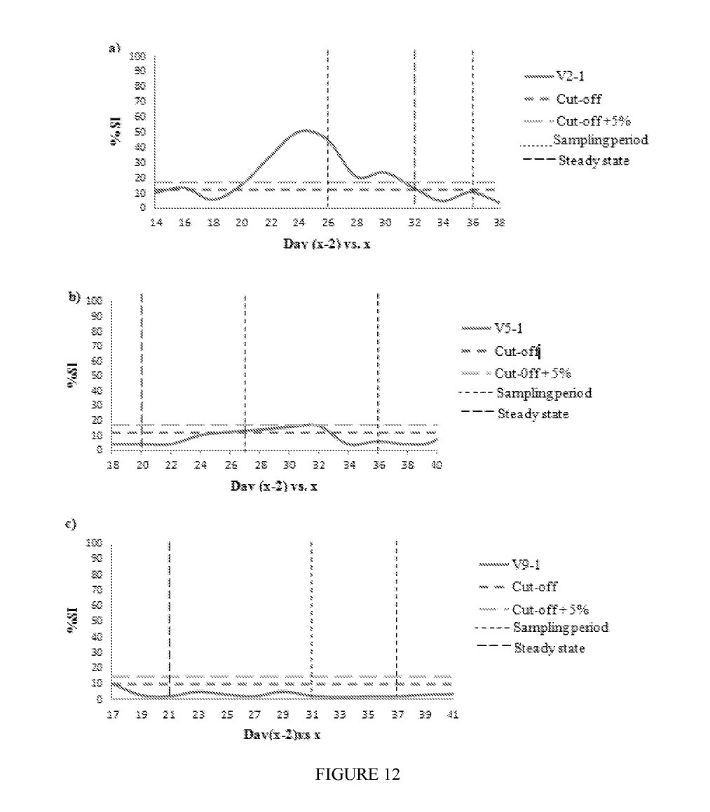

These three chemostat runs were assessed for changes in community diversity using DGGE of amplified 16S rRNA gene profiles and subsequent analysis of profiles using moving window analyses, in order to confirm steady state equilibrium was obtained and that the community was suitable for use in in vitro, small-scale, batch fermentations. A similar DGGE analysis procedure was used and showed that the greatest rate of change (Δt) values occurred between days 0-18, and stabilized by day 36 as establishment of a steady state community occurred. Given this finding, approximately the first two weeks of each chemostat run were omitted from the analysis. Varying periods of time were required for the Δt values for each vessel to drop below the individual gel-specific cut-off thresholds indicating a steady state had been obtained. However, for all vessels equilibrium was achieved by day 32 (FIG. 12a, b, c). After reaching steady state, the Δt values for all three runs stayed below or within 5% of the independent gel-specific cut-off thresholds indicating a high degree of community similarity within each vessel until the end of the analysis period on days 38 (V2-1), 40 (V5-1), and 41 (V9-1) (FIG. 12a, b, c).

FIG. 12 shows Community dynamics of chemostat runs seeded with feces from three healthy donors (donors 2, 5, and 9). Samples were analyzed every two days until completion of the small scale batch fermentations. Community dynamics were calculated using moving window correlation analysis. a) Donor 2 (days 14-38), b) Donor 5 (days 18-40), c) Donor 9 (days 17-41).

| | | | | | | | | |
|---|---|---|---|---|---|---|---|---|
| | Inoculum | Inoculum | | | | 0 vs. 48 h- | Treatment vs. | Treatment vs. |
| | Control | Control | Treatment | Treatment | 0 vs. 48 h- | Inoculum | Inoculum | Inoculum |
| Starch Substrate | 0 h | 48 h | 0 h | 48 h | Treatment | control | Control 0 h | Control 48 h |

DONOR 9

| | | | | | | | | |
|---|---|---|---|---|---|---|---|---|
| Cg102 | 88.4^{a} | 90.6^{a} | 91.2 ± 4.1^{a} | 80.6 ± 15.7^{b} | 65.9 ± 14.0 | 66.4 ± 13.4 | 95.5 ± 2.4^{a} | 53.6 ± 18.7 |
| Cg102wx | 90.9^{a} | 85.9^{a} | 87.5 ± 7.9^{a} | 90.7 ± 40.1^{a} | 66.2 ± 8.1 | 67.1 ± 10.6 | 94.6 ± 6.0^{a} | 70.3 ± 4.1 |
| Cg102ae1-Elmore | 97.6^{a} | 85.0^{a} | 95.4 ± 2.2^{a} | 94.6 ± 3.1^{a} | 52.9 ± 4.6 | 62.3 ± 3.9 | 97.0 ± 2.7^{a} | 53.4 ± 3.9 |
| Cg102ae1-ref | 94.9^{a} | 88.2^{a} | 91.3 ± 4.1^{a} | 90.6 ± 5.5^{a} | 67.7 ± 1.2 | 52.8 ± 0.3 | 95.4 ± 4.3^{a} | 57.1 ± 5.6 |
| Cgx333 | 88.8^{a} | 81.3^{b} | 90.7 ± 3.4^{a} | 90.2 ± 6.1^{a} | 65.9 ± 3.1 | 63.1 ± 6.3 | 94.2 ± 5.3^{a} | 56.2 ± 4.7 |
| Cgx333Su2 | 90.1^{a} | 95.1^{a} | 88.1 ± 6.2^{a} | 93.7 ± 3.5^{a} | 66.9 ± 2.6 | 63.0 ± 8.0 | 91.8 ± 4.0^{a} | 67.1 ± 5.3 |

DONOR 5

| | | | | | | | | |
|---|---|---|---|---|---|---|---|---|
| Cg102 | 92.5^{a} | 94.6^{a} | 92.4 ± 3.2^{a} | 94.0 ± 2.1^{a} | 69.1 ± 2.7 | 79.02 ± 0.3 | 93.3 ± 3.0^{a} | 57.9 ± 4.3 |
| Cg102wx | 95.5^{a} | 91.3^{a} | 85.8 ± 9.0^{b} | 90.6 ± 5.0^{a} | 65.7 ± 5.6 | 76.15 ± 2.4 | 91.7 ± 8.2^{a} | 50.1 ± 2.6 |
| Cg102ae1-Elmore | 78.4 | 83.0^{b} | 91.3 ± 5.0^{a} | 83.3 ± 8.8^{a} | 69.7 ± 4.9 | 85.64 ± 1.9^{b} | 90.8 ± 5.4^{a} | 61.5 ± 5.9 |
| Cg102ae1-ref | 85.4^{a} | 92.2^{a} | 83.5 ± 9.1^{b} | 86.2 ± 7.5^{a} | 69.0 ± 2.2 | 82.97 ± 2.6^{b} | 87.3 ± 11.2^{a} | 63.6 ± 5.9 |
| Cgx333 | 87.7^{b} | 74.6 | 89.8 ± 7.2^{b} | 90.3 ± 5.0^{a} | 74.4 ± 5.6 | 76.20 ± 0.9 | 88.9 ± 2.5^{b} | 61.9 ± 8.5 |
| CgxSu2 | 89.0^{a} | 91.8^{a} | 88.6 ± 8.5^{b} | 86.8 ± 5.3^{b} | 73.6 ± 5.0 | 80.67 ± 0.6 | 94.2 ± 4.7^{a} | 65.0 ± 3.6 |

DONOR 2

| | | | | | | | | |
|---|---|---|---|---|---|---|---|---|
| Cg102 | 37.3 | 73.9 | 66.1 ± 21.1 | 78.6 ± 11.6 | 79.9 ± 3.1 | 64.6 ± 28.5 | 69.5 ± 26.4 | 75.6 ± 20.7 |
| Cg102wx | 52.5 | 70.4 | 69.6 ± 19.3 | 89.9 ± 6.3^{a} | 65.8 ± 17.8 | 59.0 ± 34.2 | 74.3 ± 21.2 | 75.4 ± 18.0 |
| Cg102ae1-Elmore | 81.2^{b} | 83.2^{b} | 81.4 ± 10.2^{b} | 74.7 ± 16.5 | 73.9 ± 7.3 | 36.5 ± 7.0 | 84.9 ± 8.7^{b} | 39.9 ± 5.5 |
| Cg102ae1-ref | 25.1 | 26.5 | 48.9 ± 36.5 | 88.9 ± 6.8^{b} | 69.4 ± 24.2 | 53.0 ± 42.5 | 61.1 ± 38.1 | 63.4 ± 36.0 |
| Cgx333 | 51.6 | 55.1 | 69.2 ± 21.1 | 74.6 ± 18.0 | 81.3 ± 0.8 | 40.4 ± 15.7 | 70.3 ± 20.6 | 42.8 ± 12.9 |
| Cgx333Su2 | 53.1 | 48.0 | 69.7 ± 22.3 | 90.0 ± 4.7^{a} | 71.3 ± 7.4 | 38.5 ± 17.3 | 73.0 ± 20.0 | 50.6 ± 25.5 |

^{a}indicates correlation coefficients above the gel specific cut-off threshold representing samples with identical community profiles,
^{b}indicates correlation coefficients within 5% of the gel specific cut-off threshold representing samples with similar community profiles.
Values are means ± standard deviations (where appropriate)

Table 3.3 shows the average correlation coefficients (% SI) comparing microbial communities from small scale batch fermentations of pre-digested starch substrates inoculated with chemostat material from donor 2 (V2-1), donor 5 (V5-1), donor 9 (V9-1) at 0 and 48 hours post inoculation.

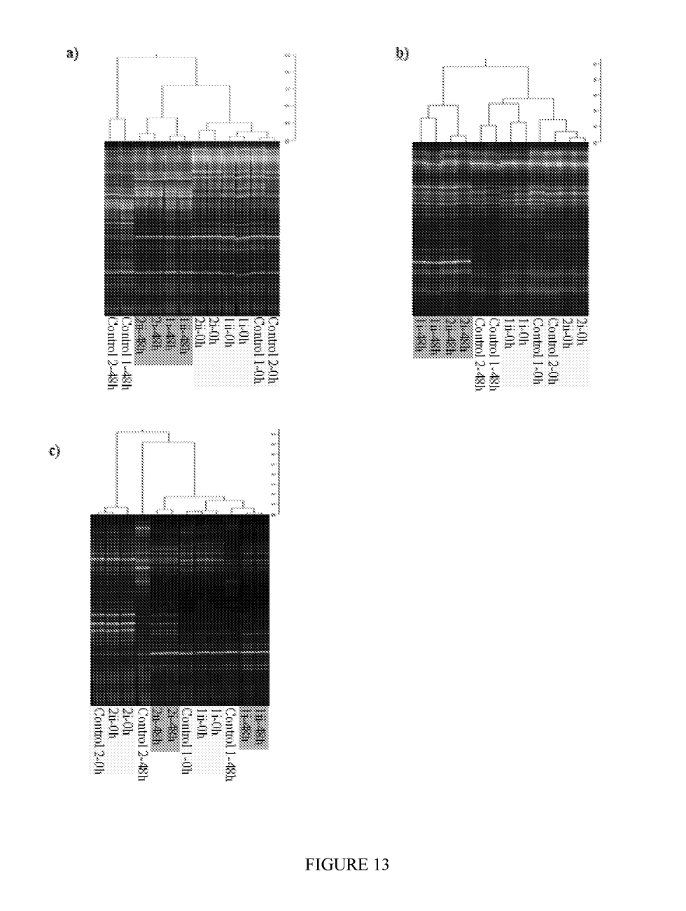

FIG. 13 Dendrograms based on Pearson and UPGMA correlation of the DGGE profiles comparing microbial communities from small scale batch fermentations of Cg102ae1-ref inoculated with chemostat material from a) donor 9 (V9-1), b) donor 5 (V5-1), c) donor 2 (V2-1), sampled at 0 and 48 hours post inoculation. Samples shaded in yellow represent clusters of 0 h samples, while samples shaded in blue represent samples containing Cg102ae-ref after 48 h of fermentation.

Small Scale Batch Fermentations with Donor 5 Fecal Microbiota

DGGE profiles for all fermentations at 0 h and 48 h using chemostat fecal inoculum from donor 5 were analyzed to ensure repeatability between replicate fermentations. Starch Substrate fermentations containing Cg102ae1 -ref and fecal inoculum from donor 5 had correlation coefficients within 5% of the gel specific cut-off threshold or above at 0 and 48 h post inoculation. On average the 0 h samples were 83.5±9.1% similar and 48 h samples were 86.2±7.5% similar (Table 3.3). The inoculum control replicates at 0 h and 48 h were 85.4% and 92.2% similar respectively both above the gel specific cut-off threshold (Table 3.3). Following inoculation (0 h) the starch substrate and inoculum control fermentation profiles on average shared a high degree of similarity (87.3±11.2%) above the gel specific cut-off threshold. After 48 h the average profile similarities were below the gel specific cut-off threshold (63.6±5.9% similar) (Table 3.3). This indicated that all fermentations were inoculated with an identical fecal community, which was modulated in a reproducible manner in response to the starch substrates.

The average correlation coefficients for the fermentations with the other 5 starch substrates at 0 h and 48 h ranged from 85.8±9.0% to 92.4±3.2% similar and 83.3±8.8% to 94.0±2.1% similar respectively, all of these values were within 5% or above of the gel specific cut-off thresholds (Table 3.3). This demonstrated that the small scale batch fermentations were consistently reproducible between replicates. Comparable to the results observed with Cg102ae1-ref, the samples containing the 5 remaining starch substrates shared high similarity to the inoculum controls at the onset of the fermentations (0 h), while upon completion (48 h) there was a considerable difference between the community profiles compared to the control. Average correlation coefficients between the starch substrate fermentations and the respective controls at 0 h and 48 h ranged from 88.9±2.5% to 94.2±4.7% similar and 50.1±2.6% to 65.0±3.6% similar respectively, 0 h samples were within 5% or above the gel specific cut-off thresholds, while 48 h samples were consistently below the gel specific cut-off thresholds indicating that the inoculum control and the starch substrate fermentation profiles were no longer similar after 48 h of fermentation (Table 3.3).

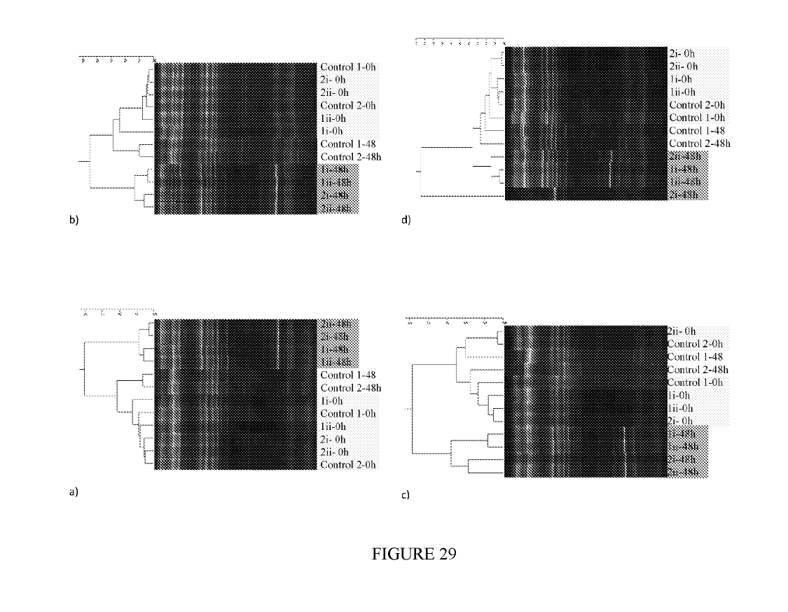

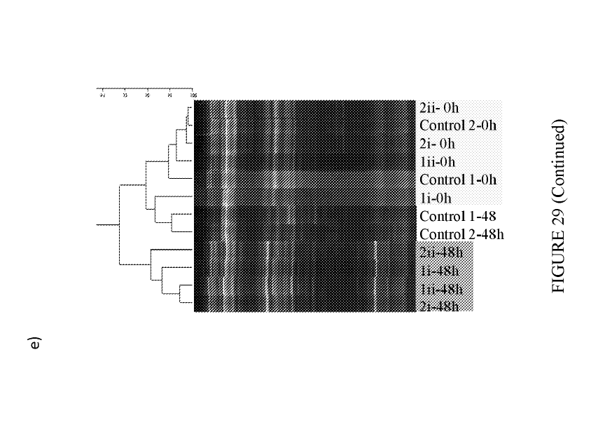

Using DGGE cluster tree analysis it was observed that all samples from the fermentation of Cg102ae1-ref (starch substrate fermentations and inoculum control) grouped together at 0 h. Samples taken after 48 h of fermentation grouped separately from those at 0 h; with starch substrate fermentations clustering together but away from the inoculum controls (FIG. 13b), indicating the sample profiles were highly similar at the onset of the fermentations and changed in response to the fermentation of Cg102ae1-ref. Similar trends were seen in the dendrograms of the fermentations with the remaining 5 starch substrates, with fermentation samples consistently clustering together but away from all other samples after 48 h of fermentation (FIG. 29).

Analysis of fermentations containing Cgx333 revealed that one 48 h sample appeared to be an outlier (2i-48 h), visual inspection of the associated DGGE profile revealed only a few bands present in the sample, and as a result the cluster tree analysis placed this sample separate from all other samples (FIG. 29). Correlation coefficients for Cgx333 donor 5 2i-48 h, when compared to all other samples, resulted in low % SI values from 12.1% to 25.5%. This anomalous result indicated that this sample was exceedingly different from all others possibly due to errors during sample collection or technical errors during DNA extraction. As such it was excluded from all calculations determining the reproducibility of the fermentations (Table 3.3).

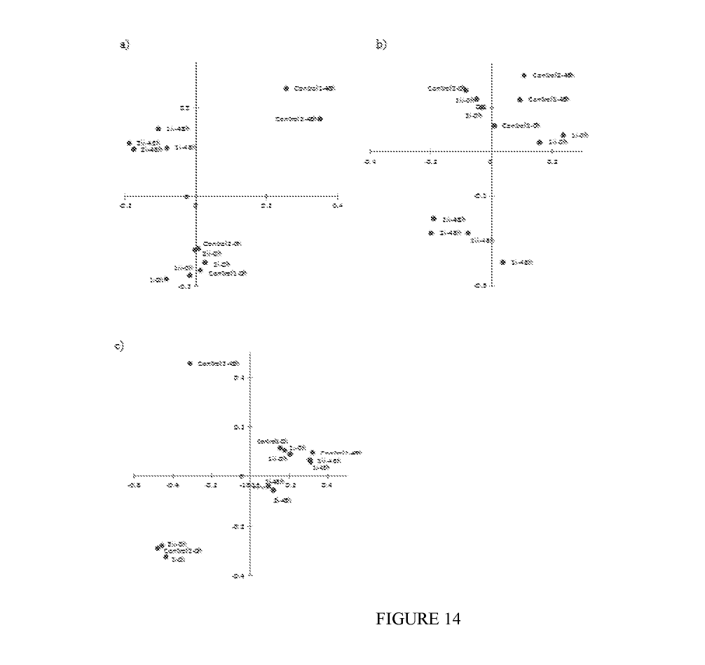

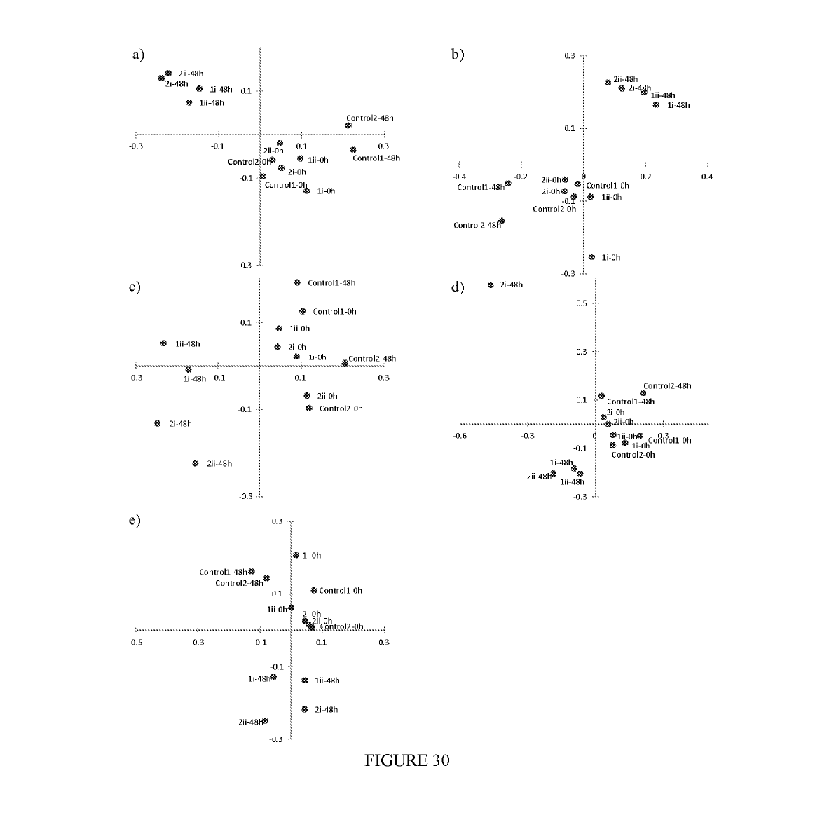

NMDS plots for all small-scale batch fermentations were created using DGGE profile similarity matrices; samples from 0 h and 48 h time points were readily distinguished from one another, as seen for example with Cg102ae1-ref (FIG. 14b) as well as the other five starch substrates (FIG. 30). The variation in the DGGE profiles of the samples was greater between time points than between sample replicates. Furthermore, a large variation in the DGGE profiles was observed between the starch substrate fermentations and inoculum controls similar to the results observed with fermentations using fecal microbiota from donor 9.

FIG. 30 shows NMDS plots of similarity matrixes generated from Pearson and UPGMA correlation of the DGGE profiles comparing microbial communities from small scale batch fermentations inoculated with chemostat material seeded with fecal microbiota from donor 9 sampled at 0 and 48 hours post inoculation a) Cg102 Kruskal's stress (1)=0.056, b) Cg102wx Kruskal's stress (1) =0.060, c) Cg102ae1-Elmore Kruskal's stress (1)=0.078, d) Cgx333 Kruskal's stress (1) =0.089, e) Cgx333Su2 Kruskal's stress (1) =0.084.

FIG. 14 NMDS plots of similarity matrixes generated from Pearson and UPGMA correlation of the DGGE profiles comparing microbial communities from small scale batch fermentations of Cg102ae1-ref sampled at 0 and 48 hours post inoculation with chemostat material from a) donor 9 (V9-1) Kruskal's stress (1) =0.073, b) donor 5 (V5-1) Kruskal's stress (1) =0.121, c) donor 2 (V2-1) Kruskal's stress (1) =0.083.

Reproducibility of Chemostat Inocula for Small Scale Batch Fermentations

The sampling period of each of the three chemostats used for the fermentations was from days 26-36 (V2-1), days 27-36 (V5-1), and days 31-37 (V9-1) (FIG. 12). To assess the reproducibility of chemostat culture as the inoculum source for in vitro small scale batch fermentations, 0 h and 48 h time points of all replicates of each starch substrate and fecal donor were compared by DGGE and subsequent analysis.

Small Scale Batch Fermentations with Donor 9 Fecal Microbiota

Profiles for all biological and technical replicates of Cg102ae1-ref incubated with fecal microbiota from donor 9 were on average 91.3±4.1% similar immediately following inoculation, and 90.6±5.5% similar after 48 h. Both of these values were above the gel specific cut-off threshold, indicating that the replicate samples were identical (Table 3.3). A comparable degree of similarity was observed between biological replicates of the inoculum control: 0 h 94.7% and 48 h 88.2% similar (Table 3.3). Comparison of Cg102ae1-ref fermentations and the inoculum control profiles were above the gel specific cut-off threshold with 95.5±2.4% similarity on average, immediately following inoculation (0 h) (Table 3.3). After 48 h, the inoculum control for donor 9 on average was 53.6±18.7% similar with the fermentations containing Cg102ae1-ref; this was well below the gel specific cut-off threshold and indicated that fermentation with Cg102ae1-ref had a distinct effect on the community dynamics (Table 3.3). DGGE cluster tree analysis displayed grouping of all samples (starch substrate fermentations and inoculum controls) together at 0 hours, indicating that the samples were highly similar prior to fermentation. 48 h samples grouped separately, with samples containing Cg102ae1-ref clustering away from the inoculum controls, further supporting that unique changes to the community dynamics had occurred in response to Cg102ae1-ref (FIG. 13a).

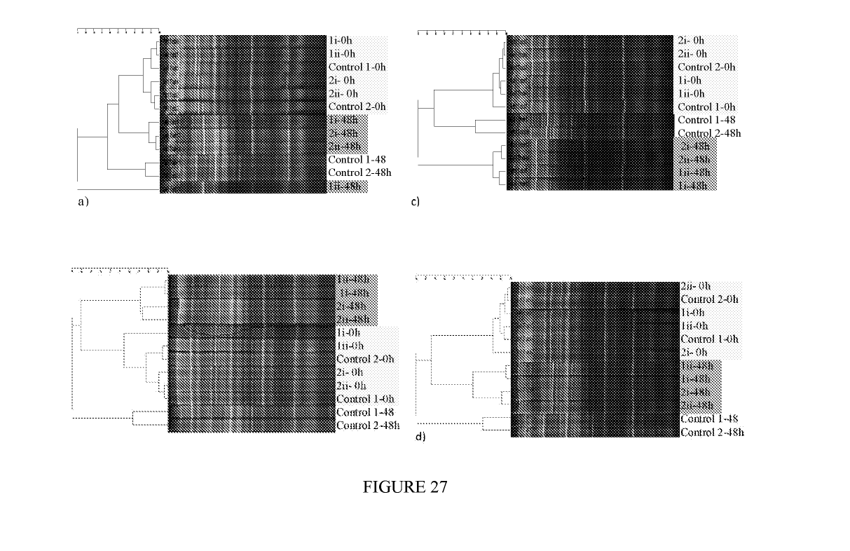

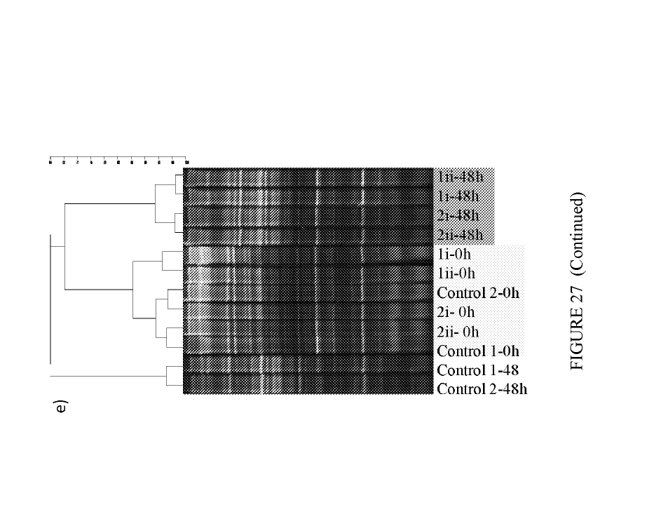

DGGE cluster tree analysis and correlation coefficients revealed similar trends for the fermentations with the remaining 5 starch substrates using chemostat material inoculated with fecal microbiota from donor 9. Average correlation coefficients comparing treatment replicates were within 5% or above the gel specific cut-off thresholds, and ranged from 87.5±7.9% to 95.4±2.2% similar at 0 h and 80.6±15.7% to 94.6±3.1% similar at 48 h, indicating that the community dynamics of the replicates were very similar at the onset and completion of all starch substrate fermentations individually (Table 3.3). Similar to Cg102ae1-ref, all other starch substrate fermentations showed high similarity to their respective inoculum controls at the start of the fermentations (0 h); average correlation coefficient values ranged from 91.8±4.0% to 97.0±2.7% similar (Table 3.3). Changes to the community dynamics between the starch substrate fermentations and inoculum controls were observed for the remaining 5 starches, as the average correlation coefficient values were all below the gel specific cut-off thresholds and ranged from 53.4±3.9% to 70.3±4.1% similar after 48 h (Table 3.3). DGGE cluster tree analysis showed comparable trends to those seen with Cg102ae1-ref: 0 h samples grouped together while 48 h starch substrate fermentations and control samples clustered separately from one another as well as from the 0 h time points (FIG. 27).

FIG. 27 shows dendrograms based on Pearson and UPGMA correlation of the DGGE profiles comparing microbial communities from replicate small scale batch fermentations of starch substrates inoculated with chemostat material from donor 9 (V9-1) sampled at 0 and 48 hours post inoculation. a) fermentations containing Cg102, b) fermentations containing Cg102wx, c) fermentations containing Cg102ae1-Elmore, d) fermentations containing Cgx333, e) fermentations containing Cgx333Su2. Samples shaded in yellow represent clusters of 0 h samples, while samples shaded in blue represent samples containing the starch substrate after 48 h of fermentation.

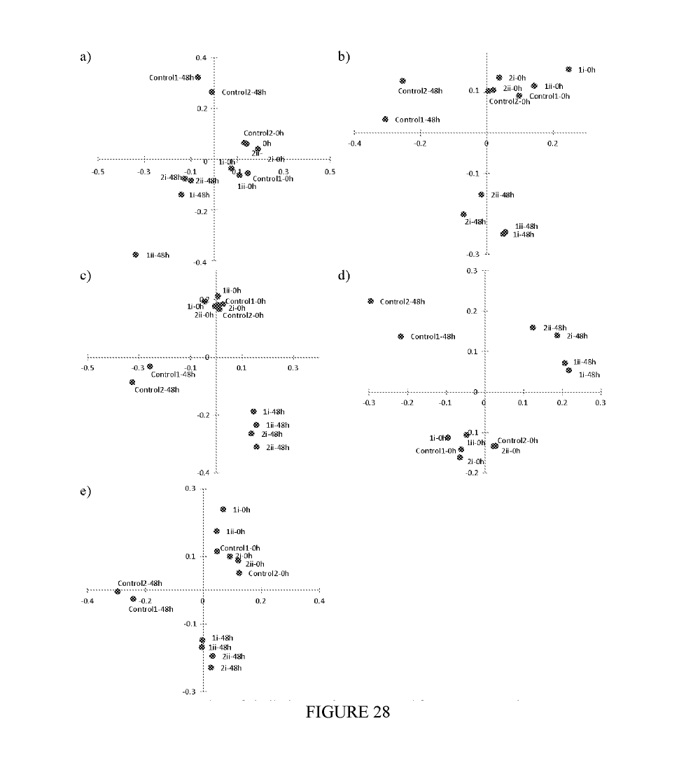

Similarity matrices were used to create non-metric multidimensional scaling (NMDS) plots for all small-scale batch fermentations. The DGGE profiles from the fermentation of Cg102ae1-ref at 0 h and 48 h were readily distinguished from one another (FIG. 14a). The variation in the DGGE profiles was greater between time points than between sample replicates. Furthermore, a large variation in the DGGE profiles was observed between the starch substrate fermentations and control. A Similar trend was observed for the NMDS plots comparing DGGE profiles of the remaining 5 starch substrates (FIG. 28).

FIG. 28 shows NMDS plots of similarity matrixes generated from Pearson and UPGMA correlation of the DGGE profiles comparing microbial communities from small scale batch fermentations inoculated with chemostat material seeded with fecal microbiota from donor 9 sampled at 0 and 48 hours post inoculation a) Cg102 Kruskal's stress (1) =0.041, b) Cg102wx Kruskal's stress (1) =0.065, c) Cg102ae1-Elmore Kruskal's stress (1) =0.041, d) Cgx333 Kruskal's stress (1) =0.084, e) Cgx333Su2 Kruskal's stress (1) =0.064.

Small Scale Batch Fermentations with Donor 5 Fecal Microbiota

DGGE profiles for all fermentations at 0 h and 48 h using chemostat fecal inoculum from donor 5 were analyzed to ensure repeatability between replicate fermentations. Starch Substrate fermentations containing Cg102ae1-ref and fecal inoculum from donor 5 had correlation coefficients within 5% of the gel specific cut-off threshold or above at 0 and 48 h post inoculation. On average the 0 h samples were 83.5±9.1% similar and 48 h samples were 86.2±7.5% similar (Table 3.3). The inoculum control replicates at 0 h and 48 h were 85.4% and 92.2% similar respectively both above the gel specific cut-off threshold (Table 3.3). Following inoculation (0 h) the starch substrate and inoculum control fermentation profiles on average shared a high degree of similarity (87.3±11.2%) above the gel specific cut-off threshold. After 48 h the average profile similarities were below the gel specific cut-off threshold (63.6±5.9% similar) (Table 3.3). This indicated that all fermentations were inoculated with an identical fecal community, which was modulated in a reproducible manner in response to the starch substrates.

The average correlation coefficients for the fermentations with the other 5 starch substrates at 0 h and 48 h ranged from 85.8±9.0% to 92.4±3.2% similar and 83.3±8.8% to 94.0±2.1% similar respectively, all of these values were within 5% or above of the gel specific cut-off thresholds (Table 3.3). This demonstrated that the small scale batch fermentations were consistently reproducible between replicates. Comparable to the results observed with Cg102ae1-ref, the samples containing the 5 remaining starch substrates shared high similarity to the inoculum controls at the onset of the fermentations (0 h), while upon completion (48 h) there was a considerable difference between the community profiles compared to the control. Average correlation coefficients between the starch substrate fermentations and the respective controls at 0 h and 48 h ranged from 88.9±2.5% to 94.2±4.7% similar and 50.1±2.6% to 65.0±3.6% similar respectively, 0 h samples were within 5% or above the gel specific cut-off thresholds, while 48 h samples were consistently below the gel specific cut-off thresholds indicating that the inoculum control and the starch substrate fermentation profiles were no longer similar after 48 h of fermentation (Table 3.3).

Using DGGE cluster tree analysis it was observed that all samples from the fermentation of Cg102ae1-ref (starch substrate fermentations and inoculum control) grouped together at 0 h. Samples taken after 48 h of fermentation grouped separately from those at 0 h; with starch substrate fermentations clustering together but away from the inoculum controls (FIG. 13b), indicating the sample profiles were highly similar at the onset of the fermentations and changed in response to the fermentation of Cg102ae1-ref. Similar trends were seen in the dendrograms of the fermentations with the remaining 5 starch substrates, with fermentation samples consistently clustering together but away from all other samples after 48 h of fermentation (FIG. 29).

Analysis of fermentations containing Cgx333 revealed that one 48 h sample appeared to be an outlier (2i-48 h), visual inspection of the associated DGGE profile revealed only a few bands present in the sample, and as a result the cluster tree analysis placed this sample separate from all other samples (FIG. 29). Correlation coefficients for Cgx333 donor 5 2i-48 h, when compared to all other samples, resulted in low % SI values from 12.1% to 25.5%. This anomalous result indicated that this sample was exceedingly different from all others possibly due to errors during sample collection or technical errors during DNA extraction. As such it was excluded from all calculations determining the reproducibility of the fermentations (Table 3.3).

FIG. 29A-E shows dendrograms based on Pearson and UPGMA correlation of the DGGE profiles comparing microbial communities from replicate small scale batch fermentations of starch substrates inoculated with chemostat material from donor 5 (V5-1) sampled at 0 and 48 hours post inoculation. a) fermentations containing Cg102, b) fermentations containing Cg102wx, c) fermentations containing Cg102ae1-Elmore, d) fermentations containing Cgx333, e) fermentations containing Cgx333Su2. Samples shaded in yellow represent clusters of 0 h samples, while samples shaded in blue represent samples containing the starch substrate after 48 h of fermentation.

NMDS plots for all small-scale batch fermentations were created using DGGE profile similarity matrices; samples from 0 h and 48 h time points were readily distinguished from one another, as seen for example with Cg102ae1-ref (FIG. 14b) as well as the other five starch substrates (FIG. 30). The variation in the DGGE profiles of the samples was greater between time points than between sample replicates. Furthermore, a large variation in the DGGE profiles was observed between the starch substrate fermentations and inoculum controls similar to the results observed with fermentations using fecal microbiota from donor 9.

Small Scale Batch Fermentations with Donor 2 Fecal Microbiota

Analysis of fermentation profiles at 0 h and 48 h using chemostat material inoculated with fecal microbiota from donor 2 indicated the fermentations had much lower similarities between biological replicates than equivalent samples inoculated with fecal microbiota from donors 5 and 9. DGGE profiles of the fermentations containing Cg102ae1-ref at 0 h when only technical replicates were compared had average correlation coefficients above the gel specific cut-off threshold (95.9% similar). The average profile similarity between all fermentation replicates with Cg102ae1-ref was 48.9±36.5% similar, signifying biological replicates were considerably different at the onset of the fermentations (Table 3.3). The same result was observed comparing the inoculum control profiles of the biological replicates at 0 h, with the correlation coefficient indicating only 25.1% similarity, below the gel specific cut-off threshold (Table 3.3). These low similarity indices indicated that the inoculum changed during the course of the fermentations. Thus differences in the fecal inoculum may overshadow the effects of the starch substrates during the fermentations.

After 48 h of fermentation with Cg102ae1-ref, technical replicates were on average 97.6±2.5% similar, which was above the gel specific cut-off threshold. Similarity between biological replicates increased after 48 h of fermentation to 84.6±0.5% within 5% of the gel specific cut-off threshold, while comparison of the inoculum control profiles at 48 h remained below the gel specific cut-off threshold at 26.5% similar. The average correlation coefficient between all replicate fermentations increased over the 48 h to 88.9±6.8% within 5% of the gel specific cut-off threshold (Table 3.3). This suggests that the fermentation of the Cg102ae1-ref caused similar changes to the microbial compositions and increased the sample similarities over the 48 h of fermentations. Unlike fermentations with fecal inocula from donors 5 and 9, a low degree of similarity was observed between the starch substrate and inoculum control fermentation profiles immediately following inoculation (0 h). On average profiles were 61.1±38.1% similar, which was below of the gel specific cut-off threshold. After 48 h the average similarity remained below the cut-off at 63.4±36.0% (Table 3.3).

These results were further supported by DGGE cluster tree analysis, fermentations with Cg102ae1-ref clustered differently than that previously observed (FIG. 13c). For example, the three 0 h samples from each of the two biological replicates (2 starch substrate fermentations, 1 inoculum control) clustered together, but separately from the other biological replicate. The 48 h starch substrate fermentation samples grouped together by technical replicate into two sets of pairs, dissimilar to the clustering observed with fermentations using donors 5 and 9 fecal microbiota. This suggests that differences in the inoculum are more evident than the effects of the starch substrate fermentation when comparing the community profiles.

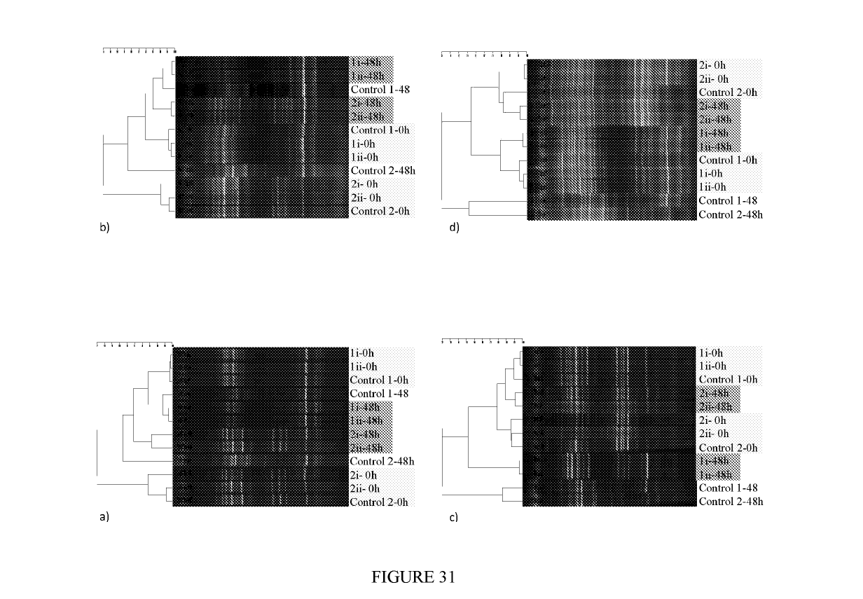

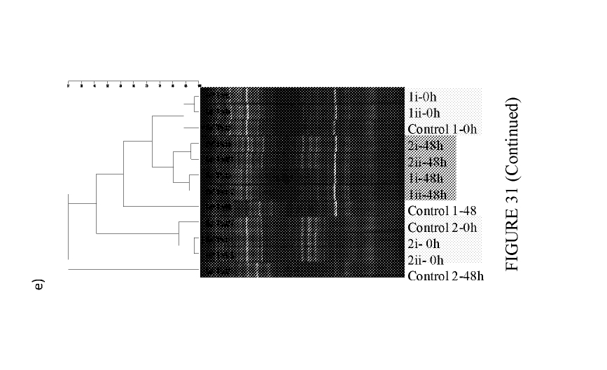

Similar results were observed upon analysis of the fermentations with the remaining 5 starch substrates. All fermentations had low similarities when comparing the 0 h sample profiles, with average correlation coefficients ranging from 66.1±21.1% to 81.4±10.2% similar, all of which were below the gel specific cut-off threshold (Table 3.3). Correlation coefficients increased for all starch substrate fermentations except Cg102ae1-Elmore after 48 h of fermentation. The values ranged from 78.6±11.6% to 90.0±4.7%, except for Cg102ae1-Elmore which dropped to 74.7±16.5% (Table 3.3). This indicated that most profiles increased in similarity in response to the starch substrates, mirroring the observations for fermentations of Cg102ae1-ref. Comparison of inoculum controls between biological replicates for each starch substrate exhibited similar results to those observed with Cg102ae1-ref. The 0 h and 48 h correlation coefficients were below or within 5% the gel specific cut-off thresholds ranging from 37.3% to 81.2% similar and 48.0% to 83.2% similar respectively(Table 3.3). This again indicated a change in the inoculum between biological replicates, similar to that seen with Cg102ae1-ref. The clustering observed within the dendrograms of the fermentations inoculated with donor 2 fecal microbiota was less consistent than that of the fermentations using the other donor's fecal microbiota. In all cases the 0 h samples created two clusters representative of the biological replicate of origin. While the 48 h samples separated into two groups according to biological replicate and clustered closer to the 0 h samples from the first biological replicate (FIG. 31A-E).

In general, these results indicate that the community dynamics of the chemostat run (V2-1) changed during the sampling period of the vessel, resulting in different communities used to inoculate the biological replicates of the fermentations. Moving window correlation analysis showed that Δt for V2-1 increased between days 28 and 32 above the gel specific cut-off threshold(FIG. 12c), falling in the middle of the sampling period indicating a rapid rate of a change in the community dynamics of the vessel. This rapid and significant change in the community structure of the chemostat corresponds with compositional differences observed between the replicates.

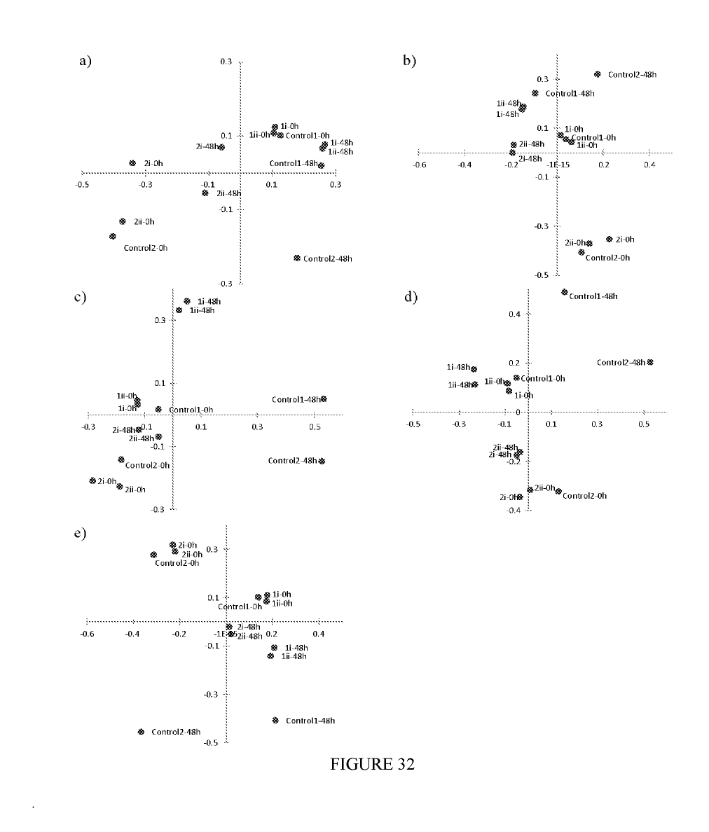

NMDS plots created using DGGE profile similarity matrices of fermentations with fecal inoculum from donor 2 were very different from those observed using fecal inoculum from donors 9 and 5. Samples from the 0 h and 48 h time points of fermentations with Cg102ae1-ref for example were readily distinguishable from one another, as were the biological replicates (FIG. 14c). This increased variability in DGGE profiles was also seen for the other five starch substrates (FIG. 32). These results consistently displayed a larger variation in the community profiles between the inoculum used for the two biological replicates as opposed to the effects of the starch substrates on the community.

FIG. 32 shows NMDS plots of similarity matrixes generated from Pearson and UPGMA correlation of the DGGE profiles comparing microbial communities from small scale batch fermentations inoculated with chemostat material seeded with fecal microbiota from donor 9 sampled at 0 and 48 hours post inoculation a) Cg102 Kruskal's stress (1) =0.086, b) Cg102wx Kruskal's stress (1) =0.110, c) Cg102ae1-Elmore Kruskal's stress (1) =0.072, d) Cgx333 Kruskal's stress (1) =0.068, e) Cgx333Su2 Kruskal's stress (1) =0.114.

Modulation of Fecal Microbiota in Response to Starch Substrates

The effects that the 6 maize substrates have on modulating the dynamics of a fecal community was evaluated with the use of three distinct chemostat runs: 1) A single-vessel inoculated with donor 2 feces, (V2-1); 2) a single-vessel inoculated with donor 5 feces, (V5-1); and 3) a single-vessel inoculated with donor 9 feces, (V9-1). The sampling of the vessels occurred from days 26-36 (V2-1), days 27-36 (V5-1), and days 31-37 (V9-1). The community dynamics of the fermentations with each starch substrate were shown to be highly reproducible between biological and technical replicates. Thus community profiles of the fermentations with all starch substrates were compared to one another at 0 h and 48 h by DGGE for each donor separately.

Modulation of Fecal Microbiota from Donor 9

Profiles of all starch substrate fermentations compared to an inoculum control were within 5% of the gel specific cut-off threshold at the onset of the fermentations (0 h) with the exception of Cg102 which had a correlation coefficient of 74.8% (Table 3.4). On average the six starch substrate fermentations were 86.9±6.2% similar to one another indicating that the microbial communities from all fermentations were very similar at the start. Average correlation coefficients between the donor control and the six starch substrate fermentations were well below the gel specific cut-off threshold after 48 h, ranging from 49.1% to 66.0% similar, indicating that considerable changes to the community dynamics occurred in response to all starch substrates (Table 3.4). DGGE profile comparisons for each of the starch substrate fermentations resulted in correlation coefficients above that of the gel specific cut-off threshold after 48 h (ranging from 89.1% to 95.4% similarity), indicating identical community changes occurred between replicates (Table 3.5). These observations supported the results previously reported in section 3.4.1 confirming the reproducibility of the fermentations inoculated with chemostat material.

TABLE 3.4
Average correlation coefficients (% SI) comparing microbial communities
from small scale batch fermentations of pre-digested starch substrates inoculated with chemostat
material seeded with donor 9 fecal microbiota (V9-1) to the inoculum control at 0 and 48 hours
post inoculation

| | Cg102 vs. | Cg102wx vs. | Cg102ae1-ref | Cg102ae1-Elmore | Cgx333 vs. | Cgx333Su2 |
|---|---|---|---|---|---|---|
| Time | inoculum | inoculum | vs. inoculum | vs. inoculum | inoculum | vs. inoculum |
| Point | control | control | control | control | control | control |
| 0 h | 74.9 | 82.0 [symbol] | 84.5^{b} | 84.0^{b} | [symbol] | 96.0 [symbol] |
| 48 h | 63.5 | 58.8 | 49.1 | 54.1 | 60.8 | 66.0 |

^{a}indicates correlation coefficients above the gel specific cut-off threshold representing samples with identical community profiles,
^{b}indicates correlation coefficients within 5% of the gel specific cut-off threshold representing samples with similar community profiles. [symbol]
indicates data missing or illegible when filed

TABLE 3.5
Average correlation coefficients (% SI) comparing microbial communities
from small scale batch fermentations of pre-digested starch substrates inoculated with chemostat
material seeded with donor 9 fecal microbiota (V9-1) 48 hours post inoculation

| Starch | | | Cg102ae1- | Cg102ae1- | | |
|---|---|---|---|---|---|---|
| Substrate | Cg102 | Cg102wx | ref | Elmore | Cgx333 | Cgx333Su2 |
| Cg102 | [symbol] | 88. [symbol] | 73.8 | 76.5 | 84. [symbol] ^{b} | 83.1^{b} |
| Cg102wx | | 89.1 [symbol] | 72.1 | 70.7 | 84.1^{b} | 82.7^{b} |
| Cg102ae1-ref | | | 95.4 [symbol] | 94.4 [symbol] | 94.6 | 68.8 |
| Cg102ae1-Elmore | | | | 95.1 [symbol] | 67.9 | 71.0 |
| Cgx333 | | | | | 95.4 [symbol] | 92.5^{a} |
| Cgx333Su2 | | | | | | 94.8 [symbol] |

^{a}indicates correlation coefficients above the gel specific cut-off threshold representing samples with identical community profiles,
^{b}indicates correlation coefficients within 5% of the gel specific cut-off threshold representing samples with similar community profiles [symbol]
indicates data missing or illegible when filed

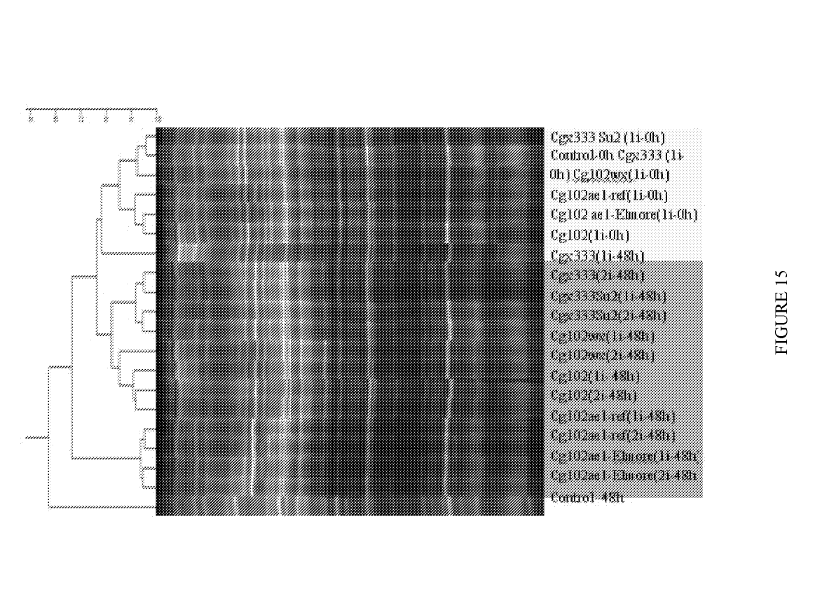

DGGE cluster tree analysis showed that all fermentations and the inoculum control clustered together immediately following inoculation (0 h). Following 48 h of fermentation the inoculum control clustered separately from all other samples, while the starch substrate fermentations clustered in pairs according to starch substrate (FIG. 15). Cg102ae1-ref and Cg102ae1-Elmore clustered more closely together, and apart from the remaining 4 starch substrates after 48 h (FIG. 15). The average correlation coefficient between Cg102ae1-ref and Cg102ae1-Elmore samples was 94.4% above the gel specific cut-off threshold, thus the two different starch substrates had similar effects on the community dynamics (Table 3.5). Cg102, Cg102wx, Cgx333, and Cgx333Su2 when compared to both Cg102ae1-ref and Cg102ae1-Elmore all had correlation coefficients that fell below the gel specific cut-off threshold, indicating the communities' dissimilarity (Table 3.5). Cg102 and Cg102wx clustered together as did Cgx333, and Cgx333Su2 with correlation coefficients above gel specific cut-off threshold, 88.17% and 92.47% respectively (FIG. 15). These four starches together formed a larger cluster with correlation coefficients ranging from 82.7% to 92.5% similar, within 5% or above the gel specific cut-off threshold indicating that fermentation of the four starch substrates resulted in communities with similar profiles.

FIG. 15 Dendrogram based on Pearson and UPGMA correlation of the DGGE profiles comparing microbial communities from small scale batch fermentations of the 6 starch substrates inoculated with chemostat material seeded with donor 9 feces (V9-1) sampled at 0 and 48 hours post inoculation. Samples shaded in yellow represent 0 h samples, while samples shaded in blue represent samples containing starch substrates after 48 h of fermentation.

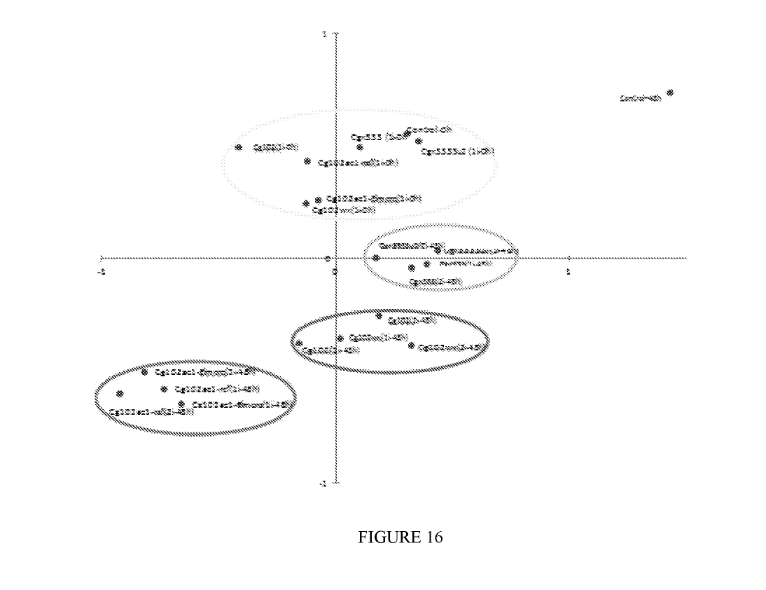

It was observed in the NMDS plots that the DGGE profiles from the fermentation of the 6 starch substrates were readily distinguishable from one another after 48 h (FIG. 16). Samples clustered on the NMDS plots in a similar manner to that observed in the dendrograms. The variation in the profiles was greater between starch substrates than between fermentation replicates, although the largest variation was observed between the sampling time points. 4 clusters were observed in the NMDS plots similar to those observed in the dendrograms: one contained all 0 h samples; the remaining 3 clusters contained the 48 h starch substrate fermentations samples. The clusters contained: 1) Cg102ae1-ref and Cg102ae1-Elmore, 2) Cg102 and Cg102wx, and 3) Cgx333 and Cgx333Su2.

FIG. 16 NMDS plots from similarity matrix generated from Pearson and UPGMA correlation of the DGGE profiles comparing microbial communities from small scale batch fermentations of starch substrates sampled at 0 and 48 hours post inoculation with chemostat material seeded with donor 9 feces (V9-1) Kruskal's stress (1)=0.105.

Modulation of Fecal Microbiota from Donor 5

Starch substrate fermentation profiles and an inoculum control profile were compared at 0 h and 48 h to determine if unique changes occurred in response to the various starch substrates. DGGE profiles from samples taken immediately following inoculation (0 h) from all fermentations were compared, profiles of the six test starches were on average 95.4±2.2% similar to one another at 0 h, which was above the gel specific cut-off threshold. This signified that all starch substrate fermentations were inoculated with an identical microbial community.

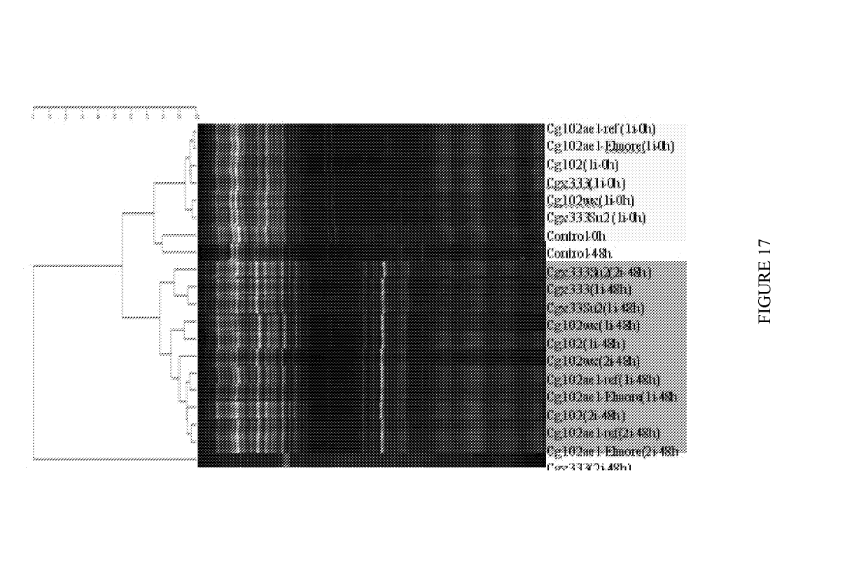

After 48 h of fermentation, when compared to the inoculum control, profiles of the six starch substrate fermentations had average correlation coefficients ranging from 36.5% to 61.8% similar, well below the gel specific cut-off threshold (Table 3.6). Therefore all starch substrate fermentations caused a significant change in the microbial community compared to that of the control. Two biological replicates for each starch were compared after 48 h of fermentation; correlation coefficients were above the gel specific cut-off threshold for all starch substrates except Cgx333 ranging from 86.2% to 96.7% (Table 3.7). The correlation coefficient between biological replicates of Cgx333 was 18.6% similar, below the gel specific cut-off threshold. This was due to Cgx333 2i-48 h, which displayed few bands on the DGGE gel (FIG. 17). As such this sample was treated as an outlier as previously discussed in section 3.4.2, potentially caused by technical error and was excluded from comparisons between the other fermentations.

TABLE 3.6

| | Cg102 vs. | Cg102wx vs. | Cg102ae1-ref | Cg102ae1-Elmore | Cgx333 vs. | Cgx333Su2 |
|---|---|---|---|---|---|---|
| Time | inoculum | inoculum | vs. inoculum | vs. inoculum | inoculum | vs. inoculum |
| Point | control | control | control | control | control | control |
| 0 h | 70.7 | 71.7 | 71.5 | 75.9 | 68.2 | 72.3 |
| 48 h | 55.3 | 36.5 | 61.8 | 60.9 | 57.0 | 59.4 |

Average correlation coefficients (% SI) comparing microbial communities from small scale batch fermentations of pre-digested starch substrates inoculated with donor 5 chemostat material (V5-1) to the inoculum control at 0 and 48 hours post inoculation

TABLE 3.7

| Starch | | | Cg102ae1- | Cg102ae1- | | |
|---|---|---|---|---|---|---|
| Substrate | Cg102 | Cg102wx | ref | Elmore | Cgx333 | Cgx333Su2 |
| Cg102 | 94.4^{a} | 88.9^{a} | 93.1^{a} | 91.6^{a} | 81.1^{b} | 73.2 |
| Cg102wx | | 86.2^{a} | 85.6^{a} | 85.0^{b} | 79.2 | 71.8 |
| Cg102ae1-ref | | | 96.7^{a} | 96.1^{a} | 89.2^{a} | 80.7 |
| Cg102ae1-Elmore | | | | 91.5^{a} | 88.9^{a} | 81.4^{b} |
| Cgx333 | | | | | 18.6* | 92.0^{a} |
| Cgx333Su2 | | | | | | 87.1^{a} |

^{a}indicates correlation coefficients above the gel specific cut-off threshold representing samples with identical community profiles,
^{b}indicates correlation coefficients within 5% of the gel specific cut-off threshold representing samples with similar community profiles.
*DGGE profile for Cgx333 2i-48 h had few to no bands as a result comparison of the two Cgx333 fermentation samples at 48 h resulted in a very low correlation coefficient. As such correlation coefficient values from comparisons with Cgx333 2i-48 h were not used in calculating means in the remainder of the table.

Table 3.7 shows average correlation coefficients (% SI) comparing microbial communities from small scale batch fermentations of pre-digested starch substrates inoculated with donor 5 chemostat material (V5-1) 48 hours post inoculation.

DGGE cluster tree analysis showed that all starch substrate fermentations clustered together into two groups based on sampling time (0 h or 48 h). The inoculum control samples (0 h and 48 h) clustered together and more closely to the 0 h cluster of the starch substrates than the 48 h cluster. The cluster containing 48 h samples was split into two subgroups, consisting of Cg102, Cg102wx, Cg102ae1-ref and Cg102ae1-Elmore and the other containing Cgx333 and Cgx333 Su2 (FIG. 17).

FIG. 17 Dendrogram based on Pearson and UPGMA correlation of the DGGE profiles comparing microbial communities from small scale batch fermentations of the 6 starch substrates inoculated with chemostat material seeded with donor 5 feces (V5-1) sampled at 0 and 48 hours post inoculation. Samples shaded in yellow represent 0 h samples, while samples shaded in blue represent samples containing starch substrates after 48 h of fermentation.

Average correlation coefficients comparing the profile similarities between the starch substrate fermentations after 48 h are reported in Table 3.7. Comparison of profiles from the cluster containing Cg102, Cg102wx, Cg102ae1-ref and Cg102ae1-Elmore had average correlation coefficients ranging from 85.6% to 96.1% similar, while the average correlation coefficient between Cgx333 and Cgx333 Su2 was 92.0% similar, all of which were above the gel specific cut-off threshold (Table 3.7). In contrast, profile comparisons between fermentations with Cgx333/Cgx333 Su2 and the four other starch substrates resulted in correlation coefficients ranging from 71.8% to 89.2% similar (Table 3.7). Correlation coefficients from Table 3.7suggest that profiles of Cgx333 and Cgx333 Su2 were more similar to Cg102ae1-ref and Cg102ae1-Elmore having correlation coefficients above the gel specific cut-off threshold, than Cg102 and Cg102wx with correlation coefficients below the gel specific cut-off threshold.

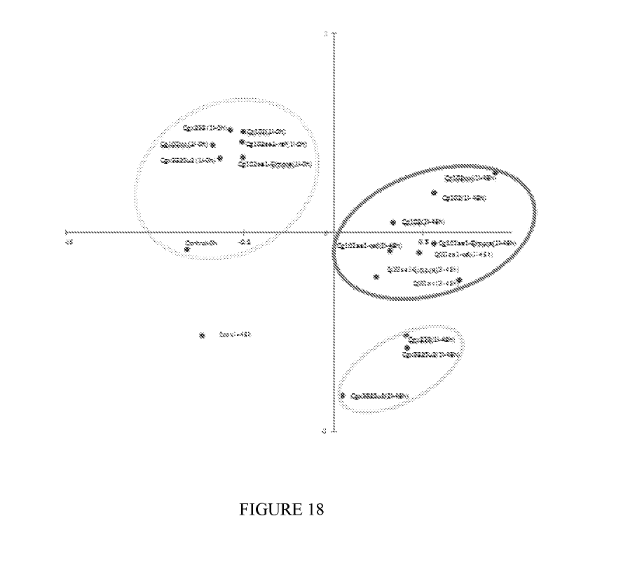

It was observed in the NMDS plots that the DGGE profiles from the fermentation of the 6 starch substrates were readily distinguishable from one another after 48 h (FIG. 18). Samples clustered on the NMDS plots in a similar manner as observed with the dendrograms. The variation in the profiles was greater between starch substrates than between fermentation replicates, although the largest variation was observed between the sampling time points.

FIG. 18 NMDS plots from similarity matrix generated from Pearson and UPGMA correlation of the DGGE profiles comparing microbial communities from small scale batch fermentations of starch substrates sampled at 0 and 48 hours post inoculation with chemostat material seeded with donor 5 feces (V5-1) Kruskal's stress (1) =0.038.

Modulation of Fecal Microbiota from Donor 2

DGGE profile comparisons between the starch substrate fermentations and the inoculum control at 0 h resulted in correlation coefficients ranging from 26.9% to 61.0% similar, below the gel specific cut-off threshold, indicating samples shared little similarity with the inoculum control at the onset of the fermentations. After 48 h the similarity between the starch substrate fermentations and the inoculum control decreased further, correlation coefficients ranged from 21.0% to 32.8% similar (Table 3.8). Correlation coefficients comparing the starch substrate fermentation profiles with one another indicated on average the starch substrate fermentations were 62.9±20.0% similar to one another immediately following inoculation (0 h), below the gel specific cut-off threshold. When DGGE profiles of the biological replicates for each starch substrate were compared after 48 h, the correlation coefficients were below the gel specific cut-off threshold for most starch substrates ranging from 62.4% to 78.9% similar. The exception was Cgx333 Su2 which had a correlation coefficient of 98.7% (Table 3.9). Therefore, all the starch substrate fermentations were dissimilar at the onset of the fermentations as were the biological replicates of each starch substrate at the conclusion of the fermentations similar to the results previously reported (section 3.4.3).

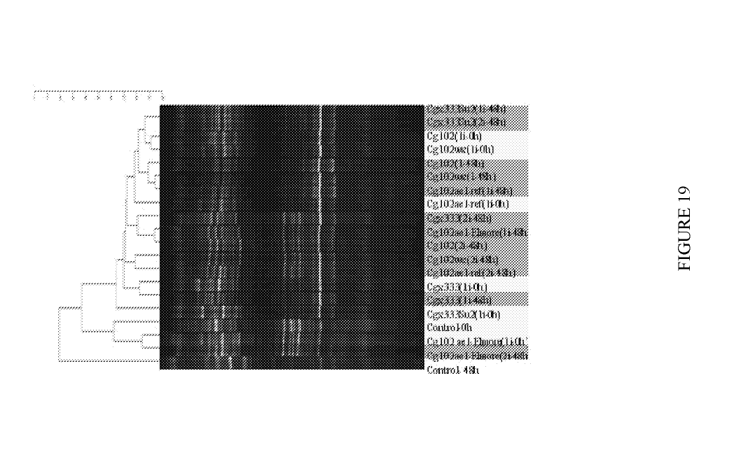

DGGE cluster tree analysis showed no clear clusters separating the 0 h and 48 h samples of donor 2, unlike that seen with the previous fermentations using fecal inoculum from donors 5 and 9. Instead, all samples appear to have clustered in a random fashion with no connections between sample time point, starch type, or biological replicate of origin (FIG. 19). A similar trend was observed analyzing correlation coefficients values comparing the different starch substrate fermentations to one another. No comparisons between two starch substrates had correlation coefficients above the gel specific cut-off threshold after 48 h, indicating no two starch substrate fermentations were similar to one another (Table 3.9).

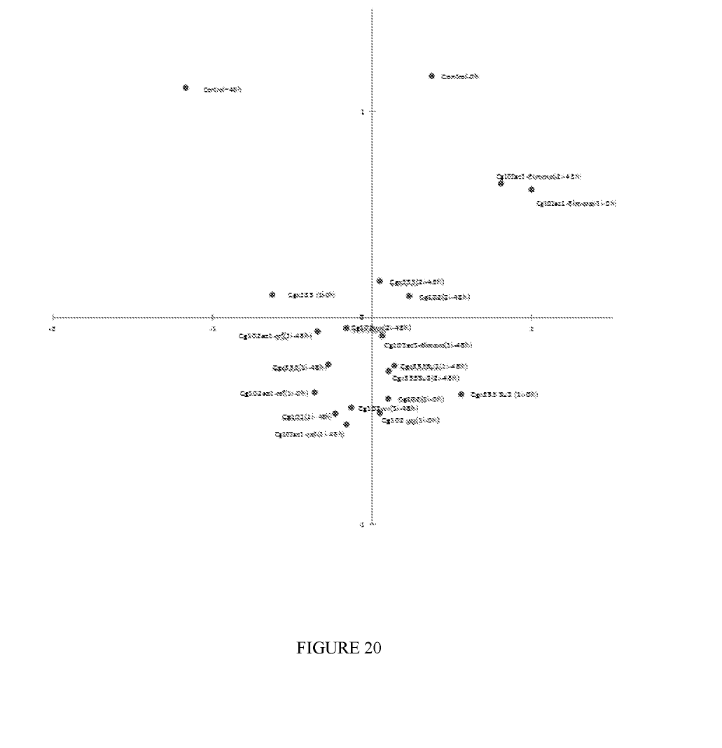

Similar results were obtained from NMDS plot produced using Pearson correlation coefficient values as that seen with the dendrograms. Although the NMDS plot was a reliable model of data with a low stress (Kruskal's stress (1) =0.113) no conclusions could be made as the data appeared to be randomly scattered across the plot (FIG. 20).

FIG. 20 NMDS plots from similarity matrix generated from Pearson and UPGMA correlation of the DGGE profiles comparing microbial communities from small scale batch fermentations of starch substrates sampled at 0 and 48 hours post inoculation with chemostat material seeded with donor 2 feces (V2-1) Kruskal's stress (1)=0.133.

TABLE 3.8
Average correlation coefficients (% SI) comparing microbial communities
from small scale batch fermentations of pre-digested starch substrates inoculated with donor 2
chemostat material (V2-1) to inoculum control at 48 hours post inoculation.

| | Cg102 vs. | Cg102wx vs. | Cg102ae1-ref | Cg102ae1-Elmore | Cgx333 vs. | Cgx333Su2 |
|---|---|---|---|---|---|---|
| Time | inoculum | inoculum | vs. inoculum | vs. inoculum | inoculum | vs. inoculum |
| Point | control | control | control | control | control | control |
| 0 h | 30.8 | 26.9 | 27.9 | 61.0 | 38.9 | 27.9 |
| 48 h | 30.2 | 21.2 | 21.0 | 23.5 | 27.1 | 32.8 |

PhAST Blue—Live/Dead Cell DNA Labeling

Analysis of the microbial communities present in the small scale batch fermentations may have been skewed due to the presence of DNA originating from dead cells. As such we decided to analyse community profiles of the fermentations with donor 9 fecal inoculum following treatment with the PhAST BLUE system. PhAST BLUE is a commercial kit that leverages the inability of DNA that has incorporated ethidium monoazide (EMA) to be amplified. Samples from microbial community sources may contain dead or dying cells, the DNA of which may skew results. Treatment of samples with EMA, and subsequent fixing with intense blue light, prior to gDNA extraction and subsequent amplification reduces the skew from microbial community profiling experiments. Unfortunately, the pHAST BLUE system only became available for my use towards the end of the project and as such only the second biological replicates of fermentations with donor 9 fecal inoculum were analysed with this technique. A limitation of the system is that the EMA treatment/light fixation step must be carried out on freshly obtained samples, undamaged by freezing or other methods of preservation.

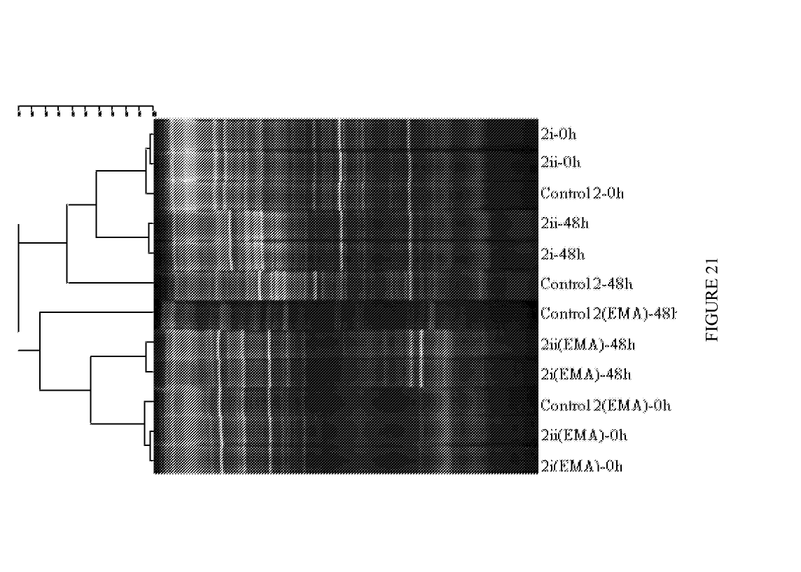

DGGE profiles from samples at 0 h and 48 h were compared with and without EMA treatment for each of the six starch substrate fermentations. Correlation coefficients from profile comparisons of Cg102ae1-ref fermentations and the inoculum control at 0 h were on average 97.4% similar, the same samples treated with the EMA had profiles that were on average 96.3% similar (Table 3.10). EMA treated and untreated samples at 0 h had an average similarity of 26.5% (Table 3.11). After 48 hours of fermentation, profiles of EMA treated samples containing Cg102ae1-ref were 96.7% similar and on average 29.3% similar to the inoculum control treated with EMA (Table 3.10). Profiles between Cg102ae1-ref fermentations without EMA treatment were 97.5% similar 48 h post inoculation, and on average were 52.3% similar to the inoculum control after 48 h (Table 3.10). Comparisons of the DGGE profiles from paired 48 h Cg102ae1-ref fermentation samples (EMA treated vs. untreated) were on average 23.5% similar (Table 3.11). These results indicate that the PhAST BLUE system reproducibly inactivated DNA from dead cells, as replicates at 0 h and 48 h were above the gel specific cut-off threshold and therefore identical both with and without EMA treatment. Furthermore, it was observed at both the 0 h and 48 h time points that there were substantial differences between the community profiles after samples were treated with EMA; this indicated a significant contribution to the profiles due to DNA originating from dead cells. DGGE cluster tree analysis resulted in four distinct clusters based on sample time point and EMA treatment, with the exception of the 48 h inoculum controls (with and without EMA treatment) which both independently clustered separately from all other samples (FIG. 21).

Table 3.10 Average correlation coefficients (% SI) comparing the reproducibility of microbial communities from replicate small scale batch fermentations of pre-digested starch substrates inoculated with donor 9 chemostat material (V9-1). Samples were taken at 0 and 48 hours post inoculation, values are presented either with (shaded) or without EMA treatment.

TABLE 3:11
Average correlation coefficients (% SI) from small scale batch
fermentations of pre-digested starch substrates inoculated with
donor 9 chemostat material (V9-1), comparing the similarity between
microbial communities taken 0 and 48 hrs post inoculation
presented as (% SI) values between samples with or without
EMA treatment.

| | EMA and | EMA and | EMA and |
|---|---|---|---|
| | untreated | untreated | untreated |
| | fermentations | fermentations | inoculum control |
| Starch Substrate | at 0 h | at 48 h | 48 h |
| Cg102 | 36.4 | 39.4 | 34.2 |
| Cg102wx | 49.2 | 48.6 | 21.6 |
| Cg102ae1-ref | 26.5 | 23.5 | 21.6 |
| Cg102ae1-Elmore | 30.7 | 32.7 | 40.8 |
| Cgx333 | 17.7 | 13.2 | 20.5 |
| Cgx333Su2 | 47.2 | 43.7 | 40.5 |

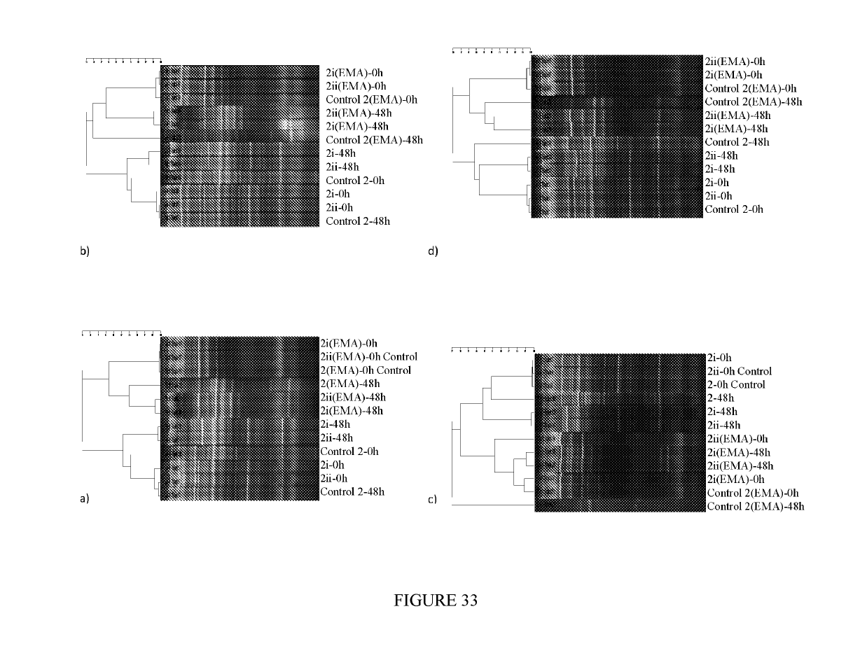

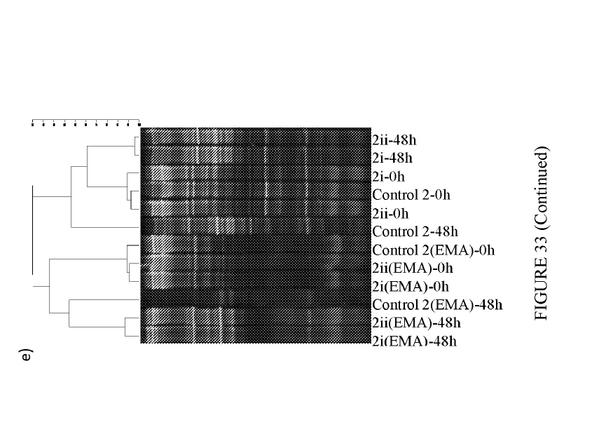

A comparable trend was seen with the remaining five starch substrates to that seen with Cg102ae1-ref, in that EMA treatment consistently neutralized DNA from dead cells. This resulted in DGGE profiles of replicate samples maintaining high levels of similarity at both 0 h and 48 h, as observed with untreated samples (Table 3.10). Average correlation coefficients comparing DGGE profiles at 0 h and 48 h with and without EMA treatment are reported in (Table 3.11). DGGE cluster tree analysis of the five starches resulted in similar clustering patterns as that observed with Cg102ae1-ref (FIG. 33).

FIG. 33A-E Dendrograms based on Pearson and UPGMA correlation of the DGGE profiles comparing microbial communities pre and post EMA treatment. Sampled 0 and 48 hours post inoculation from replicate small scale batch fermentations of starch substrates inoculated with chemostat material donor 9 (V9-1). a) fermentations containing Cg102, b) fermentations containing Cg102wx, c) fermentations containing Cg102ae1-Elmore, d) fermentations containing Cgx333, e) fermentations containing Cgx333Su2.

TABLE 3.9
Average correlation coefficients (% SI) comparing microbial communities
from small scale batch fermentations of pre-digested starch substrates inoculated with donor 2
chemostat material (V2-1) 48 hours post inoculation.

| Starch | | | Cg102ae1- | Cg102ae1- | | |
|---|---|---|---|---|---|---|
| Substrate | Cg102 | Cg102wx | ref | Elmore | Cgx333 | Cgx333Su2 |
| Cg102 | 71.1 | 76.7 | 73.2 | 68.2 | 76.1 | 83.9 |
| Cg102wx | | 78.9 | 82.8 | 58.4 | 76.9 | 78.3 |
| Cg102ae1-ref | | | 74.3 | 55.8 | 75.8 | 74.4 |
| Cg102ae1-Elmore | | | | 62.4 | 67.7 | 72.9 |
| Cgx333 | | | | | 78.2 | 76.8 |
| Cgx333Su2 | | | | | | 98.7^{a} |

^{a}indicates correlation coefficients above the gel specific cut-off threshold representing samples with identical community profiles,
^{b}indicates correlation coefficients within 5% of the gel specific cut-off threshold representing samples with similar community profiles.

FIG. 19 Dendrogram based on Pearson and UPGMA correlation of the DGGE profiles comparing microbial communities from small scale batch fermentations of the 6 starch substrates inoculated with chemostat material seeded with donor 2 feces (V2-1) sampled at 0 and 48 hours post inoculation. Samples shaded in yellow represent 0 h samples, while samples shaded in blue represent samples containing starch substrates after 48 h of fermentation.

GC-MS Data Analysis

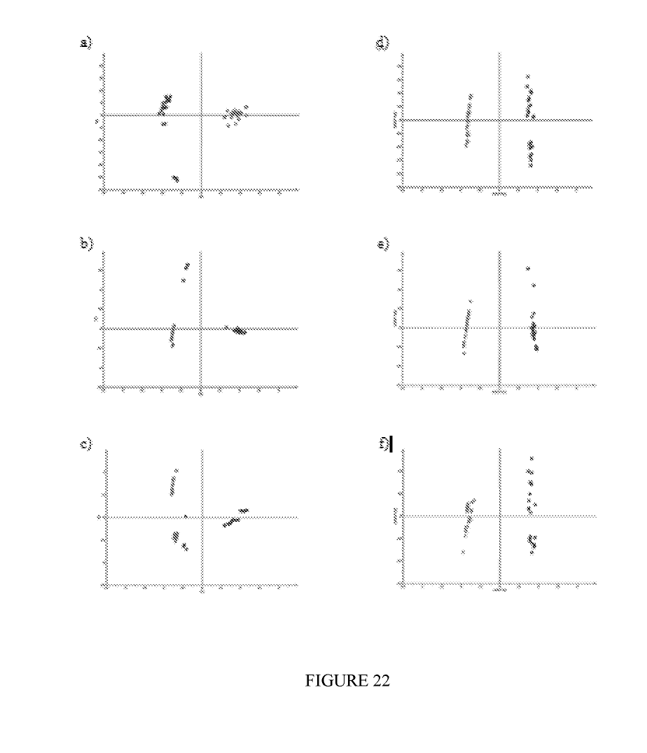

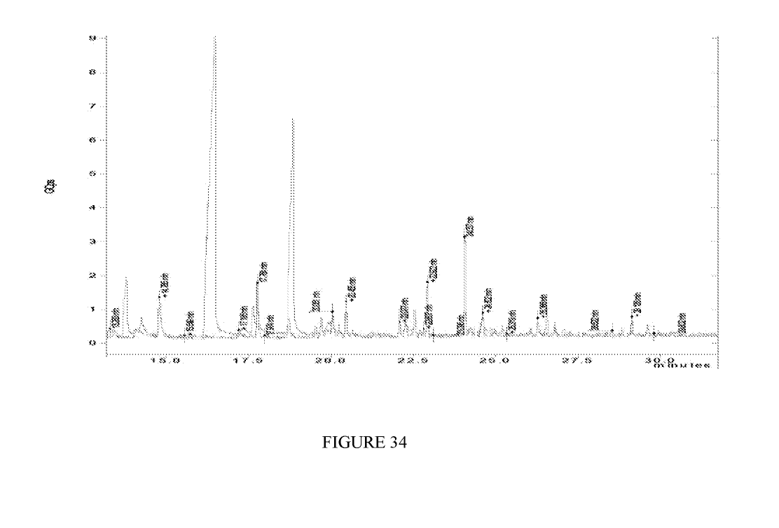

Visual inspection of GC/MS chromatograms showed differences between the 0 h and 48 h starch substrate fermentation samples (FIG. 34). PCA models were constructed using the GC/MS data of the fermentations inoculated with fecal microbiota from each of the three donors individually to visualize trends in the data and identify outliers. The PCA models consistently separated the 0 h samples from the 48 hour starch substrate fermentation samples primarily along the first principal component (PC) t[1] (FIG. 22a-c). Furthermore, the 48 h inoculum control samples clustered separately in all three PCA models. A single outlier, Cgx333(2i-48 h), was identified within the dataset for fermentations with fecal inoculum from donor 5 and was omitted from all subsequent analyses as it clustered together with the 0 h samples opposed to the 48 h samples. OPLS-DA models were constructed to identify potential variables differing between the 0 and 48 h sample classes, control samples were removed for better identification of variables influenced by the fermentation of the starch substrates (FIG. 32d-f). PCA is an unsupervised technique in which points are separated only by the variance within the data set; alternatively OPLS-DA is a supervised technique that utilizes class identity, in this case sampling time, in a Y matrix and correlates this to the data obtain from the GC-MS analysis. The data that discriminates between the two defined classes is forced into the first PC while data that is not contributing to the class separation is placed into successive orthogonal components. The OPLS-DA models constructed from the data sets of the three donors separated all of the 0 h fermentation samples from the 48 h samples along the first PC.

FIG. 34 shows total ion chromatograms from the fermentation of Cg102ae1-ref with chemostat material inoculated with fecal microbiota from donor 9 at the 0 h and 48 h time points. Chromatogram in red represents the 0 h time point while the Chromatogram in green represents the 48 h time point.

VIP plots were used for the identification of variables responsible for group separation. Variables were identified using VIP statistics (VIP >1) as having the largest impact on the separation of the two classes, statistically significant differences between the 0 and 48 h time points of the identified variables was confirmed using the Mann-Whitney-Wilcoxon test on the normalized peak areas. Results for each variable along with tentative metabolite identifications and fold changes are reported for fermentations with fecal microbiota from donors 9, 5, and 2 in Tables 3.12, 3.13 and 3.14 respectively. The majority of metabolites identified as differing between the 0 and 48 h samples showed a decrease over the fermentation period. Since the aim of this study was to identify metabolites produced by the fecal microbiota which might be available for absorption by the host, metabolites with associated decreases were not of particular interest to this work and are not discussed further.

A statistically significant increase in butanoic acid was observed after 48 hours of fermentation with fecal inocula from all donors. Furthermore after 48 h, fermentations with fecal inoculum from donor 9 resulted in a significant increase in both pentanoic acid and propanoic acid (Tables 3.12, 3.13 and 3.14). The differences in the metabolites that were identified as having a significant impact on the separation of the sample classes (time points) for each donor's fecal inoculum suggests that an individual's microbiota plays a critical role in the production and availability of metabolites for the host.

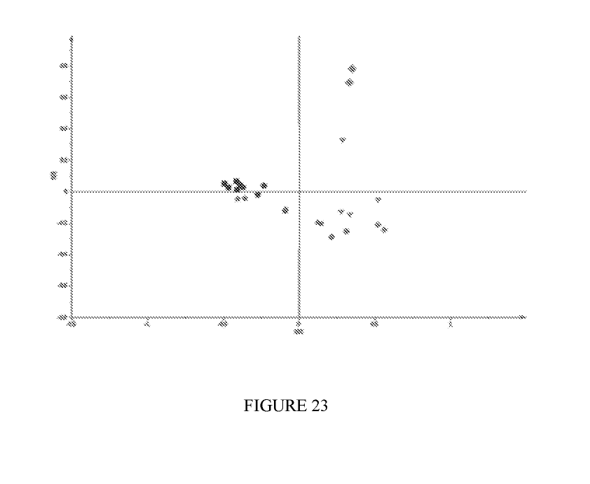

To assess whether the 3 donors fecal microbiota had unique responses to the 6 starch substrates, further analysis was completed on a subset of the previously analysed datasets. GC-MS data pertaining to only the 48 h samples was separated into 6 classes based on starch substrate and analysed using both PCA and OPLS-DA models. The PCA model for the fermentations with fecal inoculum originating from donor 9 indicated a trend towards separating the 6 starch substrates along the first PC (FIG. 23); however this trend was not observed in the PCA models for the other two donors (data not shown) and no further analysis was carried out. OPLS-DA models were generated for the analysis of the 48 h fermentation samples for donor 9 comparing two starch substrates at a time. No significant models could be derived by OPLS-DA when comparing the differences between all possible pairwise combinations of the starch substrates. All models generated resulted in low R2Y(cum) and Q2(cum) values with CV-ANOVA p-values >0.05(data not shown), therefore the models were over fitting the data and were also not significant due to the high CV-ANOVA p-values. This indicated a much less pronounced difference between the starch substrates than that observed between the 0 h and 48 h time points for each donor's fecal microbiota. As such no specific metabolites could be identified as significantly different between the starch substrates.

FIG. 21 Dendrogram based on Pearson and UPGMA correlation of the DGGE profiles comparing microbial communities pre and post EMA treatment. Sampled 0 and 48 hours post inoculation from replicate small scale batch fermentations of Cg102ae1-ref inoculated with chemostat material donor 9 (V9-1).

FIG. 22 PCA models (panels a-c) and OPLS-DA models (panels d-f) of GC/MS data obtained from starch substrate fermentations with chemostat material from donor 9 (panels a and d), donor 5(panels b and e), and donor 2 (panels c and f). Variables are mean-centered and pareto-scaled, for OPLS-DA models 0 and 48 h time points were used as the discriminating Y matrix. Model characteristics are as follows: (a) R²X(cum) 0.902, Q² (cum) 0.825, and nine significant PCs; (b) R²X(cum) 0.933, Q² (cum) 0.82, and seven significant PCs; (c) R2X(cum) 0.84, Q² (cum) 0.762, and three significant PCs; (d) Significant components 1+1, R²X(cum) 0.802, R²Y(cum) 0.998, Q² (cum) 0.995, CV ANOVA 0; (e) Significant components 1+1, R²X(cum) 0.826, R²Y(cum) 0.997, Q² (cum) 0.995, CV ANOVA 0;(f) Significant components 1+1, R²X(cum) 0.7, R²Y(cum) 0.995, Q² (cum) 0.992, CV ANOVA 0. Figure key: green circles are 0 h fermentation samples, blue squares are 48 h fermentation samples.

TABLE 3.12
Mass retention time pairs of metabolic changes from the fermentation
of starch substrates with donor 9 chemostat material over 48 hours, identified
using SPME-GC/MS

| | Variable ID | 0 h Average | 48 h Average | |
|---|---|---|---|---|
| | [M (m/z) T | Normalized Peak | Normalized Peak | Fold |
| Metabolite | (RT)] | Area | Area | Change |
| Butanoic acid | M60T981 | 0.00 | 1.62E−01 ± 5.46E− | * |
| Pentanoic acid | M60T1127 | 0.00 | 7.30E−02 ± 2.45E− | * |
| Unidentified | M207T849 | 2.88E−02 ± 1.54E− | 6.52E−03 ± 5.61E− | −4.41* |
| Heptanal | M44T1065 | 1.48E−02 ± 1.04E− | 1.09E−03 ± 1.13E− | −13.61* |
| 9,10-Anthracenedione, 1,8- | M73T1352 | 2.40E−02 ± 9.04E− | 2.96E−03 ± 1.84E− | −8.08* |
| Hexanal | M42T887 | 1.48E−02 ± 6.62E− | 1.98E−03 ± 2.83E− | −7.50* |
| Propanoic acid | M74T825 | 0.00 | 1.46E−02 ± 1.53E− | * |
| Nonanal | M44T1375 | 8.23E−03 ± 4.47E− | 1.36E−03 ± 1.27E− | −6.06* |
| Octanal | M44T1228 | 6.42E−03 ± 4.52E− | 1.03E−03 ± 1.29E− | −6.21* |
| 3-Phenyl-1-propanol, acetate | M118T1749 | 8.71E−03 ± 5.04E− | 7.39E−04 ± 1.51E− | −11.78* |
| Phenol | M94T1324 | 4.21E−03 ± 8.69E− | 9.41E−03 ± 1.45E− | 2.23 |
| (E)-2-Nonenal | M42T1476 | 9.62E−03 ± 6.32E− | 9.31E−04 ± 1.21E− | −10.33* |
| Unidentified | M42T1337 | 8.49E−03 ± 3.18E− | 8.73E−04 ± 1.18E− | −9.72* |
| 2-Methylphenol | M40T1444 | 6.72E−03 ± 1.60E− | 2.35E−03 ± 2.83E− | −2.86* |
| Benzaldehyde | M105T1203 | 6.94E−03 ± 5.64E− | 1.37E−03 ± 1.05E− | −5.06* |

Table 3.12 shows a negative fold change represents a decrease in concentration between 0 h and 48 h, while no reported fold change value indicates the metabolite was not detected at one of the two time points. Metabolites are only putatively assigned; identification was carried out by comparison to the NIST mass spectral database. Values are ±standard error with n=24, statistical significance levels were determined by Mann-Whitney-Wilcoxon test: *indicates p-value<0.05.

TABLE 3.13
Mass retention time pairs of metabolic changes from the fermentation of
starch substrates with donor 5 chemostat material over 48 hours, identified using SPME-GC/MS

| | Variable ID | 0 h Average | 48 h Average | |
|---|---|---|---|---|
| | [M (m/z) T | Normalized Peak | Normalized Peak | Fold |
| Metabolite | [symbol] | Area | Area | Change |
| Benzaldehyde | M52T1199 | 1.29E−02 ± 1.07E− | 1.43E−03 ± 1.20E− | −8.98* |
| *Unidentified | M55T1331 | 1.26E−02 ± 9.44E− | 1.97E−03 ± 1.90E− | −6.37* |
| *Unidentified | M56T852 | 8.98E−03 ± 6.42E− | 0.00 | * |
| Heptanal | M56T1062 | 1.21E−02 ± 1.16E− | 7.09E−04 ± 8.03E− | −16.98* |
| Dibutyl peroxide | M57T655 | 4.75E−02 ± 4.91E− | 8.92E−04 ± 4.58E− | −53.27* |
| Hexanal | M57T886 | 2.43E−02 ± 1.16E− | 1.80E−03 ± 1.79E− | −13.45* |
| *Unidentified | M57T1468 | 1.15E−02 ± 1.47E− | 1.88E−03 ± 2.47E− | −6.09* |
| *Unidentified | M58T1039 | 9.30E−03 ± 8.75E− | 4.18E−04 ± 5.11E− | −22.25* |
| 2-Dodecanone | M59T1051 | 6.28E−03 ± 2.95E− | 2.41E−05 ± 1.37E− | — |
| 2-Heptanone | M59T1209 | 8.76E−03 ± 4.72E− | 7.82E−04 ± 5.43E− | −11.19* |
| Ethoxyacetic acid | M60T434 | 6.63E−03 ± 7.72E− | 0.00 | * |
| Butanoic acid | M60T989 | 0.00 | 5.73E−01 ± 6.50E− | * |
| Phenol | M67T1319 | 8.06E−03 ± 4.62E− | 1.62E−03 ± 1.01E− | −4.98* |
| 2-Methylphenol | M91T1446 | 1.57E−02 ± 1.01E− | 3.56E−03 ± 3.73E− | −4.40* |
| *Unidentified | M207T848 | 3.15E−02 ± 2.46E− | 7.44E−03 ± 5.91E− | −4.23* |
| *Unidentified | M57T1184 | 6.53E−03 ± 4.28E− | 1.17E−03 ± 1.01E− | −5.56* |
| 3-Phenyl-1-propanol, | M64T1743 | 3.40E−03 ± 3.66E− | 2.60E−03 ± 3.81E− | −1.30 |

[symbol]

indicates data missing or illegible when filed

Table 3.13 shows a negative fold change represents a decrease in concentration between 0 h and 48 h, while no reported fold change value indicates the metabolite was not detected at one of the two time points. Metabolites are only putatively assigned; identification was carried out by comparison to the NIST mass spectral database. Values are ±standard error with n=24, statistical significance levels were determined Mann-Whitney-Wilcoxon test: *indicates p-value<0.05.

TABLE 3.14
Mass retention time pairs of metabolic changes from the fermentation of
starch substrates with donor 2 chemostat material over 48 hours, identified using SPME-GC/MS

| | Variable ID | 0 h Average | 48 h Average | |
|---|---|---|---|---|
| | [M (m/z) T | Normalized | Normalized Peak | Fold |
| Metabolite | [symbol] | Peak | Area | Change |
| Hexanal | M58T888 | 7.51E−03 ± 1.02E− | 5.93E−04 ± 3.75E− | −12.65* |
| Butanoic acid | M60T984 | 0.00 | 5.55E−01 ± 1.78E− | * |
| *Unidentified | M71T1191 | 6.35E−03 ± 6.98E− | 6.38E−04 ± 1.08E− | −9.95* |
| 2-Methylphenol | M80T1444 | 3.17E−02 ± 9.59E− | 7.62E−03 ± 7.98E− | −4.15* |
| Benzaldehyde | M105T1203 | 1.06E−02 ± 5.05E− | 1.88E−03 ± 9.85E− | −5.62* |
| 3-Phenyl-1-propanol, | M118T1748 | 8.82E−03 ± 8.68E− | 1.25E−03 ± 2.26E− | −7.05* |
| *Unidentified | M55T856 | 1.12E−02 ± 1.30E− | 0.00 | * |
| *Unidentified | M55T1033 | 1.08E−02 ± 1.78E− | 1.53E−03 ± 3.66E− | −7.06* |
| Heptanal | M55T1066 | 4.74E−03 ± 3.47E− | 6.08E−04 ± 4.85E− | −7.79* |
| *Unidentified | M55T1336 | 8.14E−03 ± 7.21E− | 8.33E−04 ± 1.05E− | −9.77* |
| Dibutyl peroxide | M56T658 | 1.36E−01 ± 2.33E− | 2.56E−03 ± 1.02E− | −53.10* |

[symbol]

indicates data missing or illegible when filed

Table 3.14 shows a negative fold change represents a decrease in concentration between 0 h and 48 h, while no reported fold change value indicates the metabolite was not detected at one of the two time points. Metabolites are only putatively assigned; identification was carried out by comparison to the NIST mass spectral database. Values are ±standard error with n=24, statistical significance levels were determined by Mann-Whitney-Wilcoxon test: *indicates p-value<0.05.

Chemostat Feeding Trial

In this experiment, because of the complex nature of human feeding trials, the use of chemostats was explored as an alternative: chemostat studies have previously proven to be an effective means to model the human distal colon. A starch-enriched medium was prepared for use in the chemostat feeding trials, the basal media recipe (2L) (Table 2.2) was enriched with 120 g of predigested Hi-Maize 260 (+RS) or cornstarch(+CS) resulting in the vessels being provided an additional ˜30 g of predigested starch per day for four days (see section 2.5 for details).

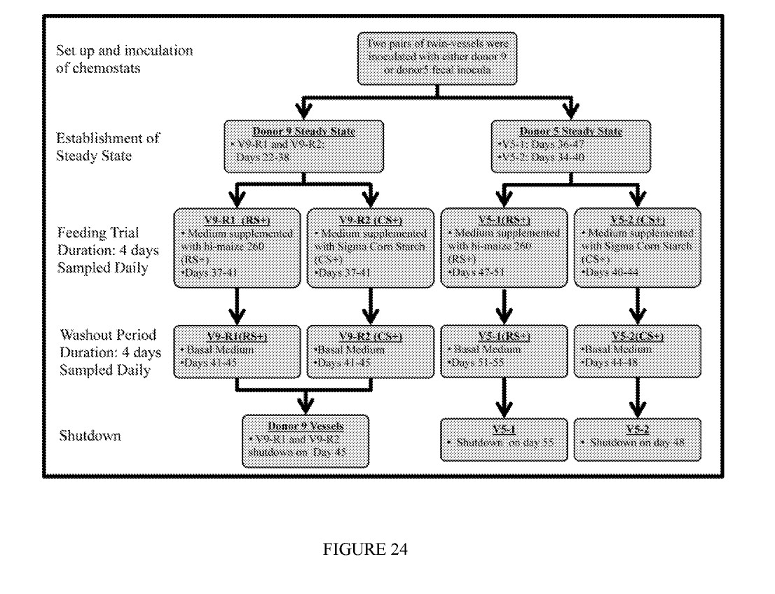

Two separate chemostat runs were analyzed in this study: 1) twin-vessels seeded with fresh feces from donor 9 and fed starch-enriched media for 4 days followed by a return to basal medium for 4 days (V9-R1 and V9-R2); and 2) twin-vessels seeded with donor 5 feces and fed starch-enriched media for 4 days followed by a return to basal medium for 4 days (V5-1 and V5-2). One vessel from the chemostat run inoculated with fecal microbiota from donor 5 was being used for another unrelated experiment, because of this the feeding trial for each of the twin vessels was initiated on different days. Additional analysis was done to ensure significant changes did not occur to V5-2 during the seven days between the initiations of the feeding trials. FIG. 24 outlines the timeline and work flow of the feeding trial experiment.

Twin-vessels from the two separate chemostat runs were analyzed by DGGE to determine whether starch enriched medium containing predigested resistant starch (+RS) compared to predigested corn starch (+CS) affected the community dynamics and stability of simulated distal gut communities.

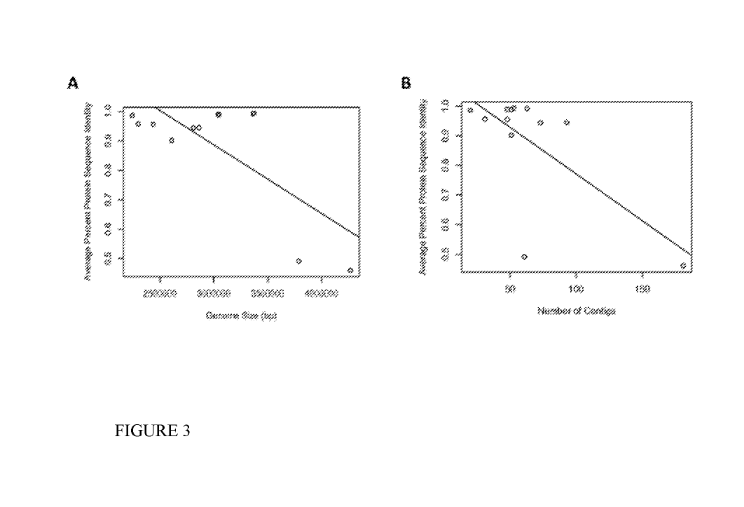

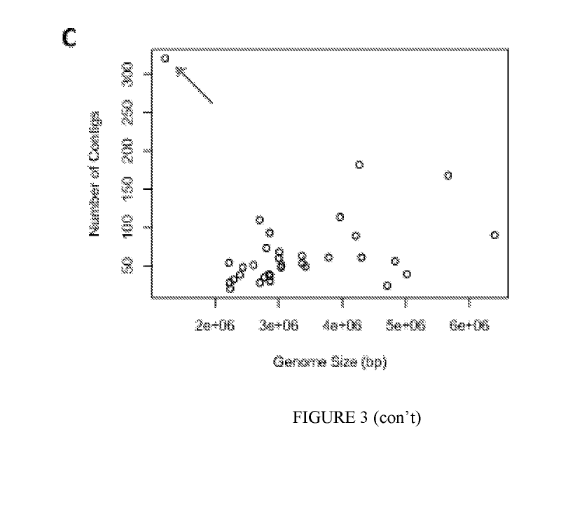

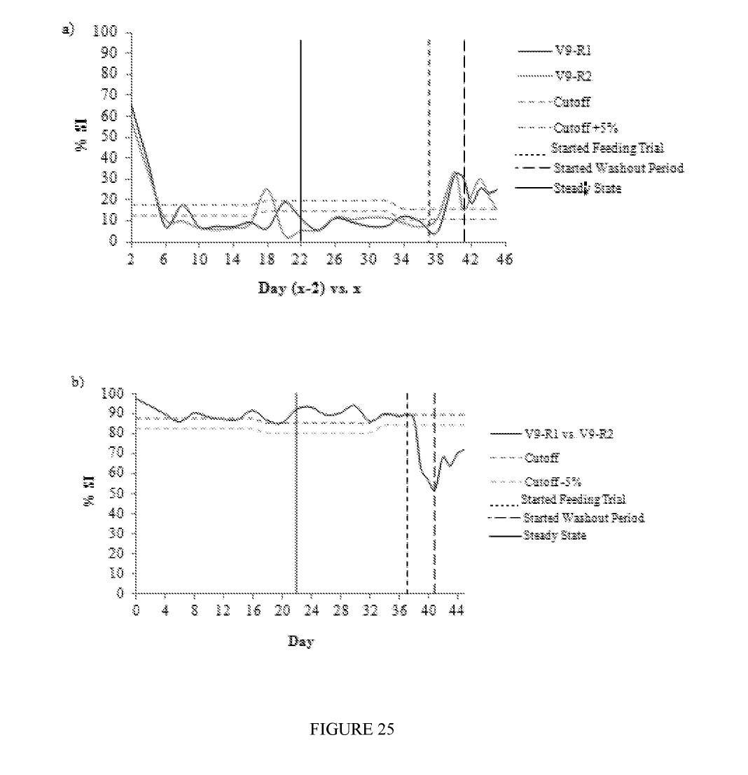

Moving window correlation analysis for V9-R1 and V9-R2 resulted in reproducible rate-of-change (Δt) values below the gel specific cut-off thresholds between days 22-38 for both vessels (FIG. 25a). V5-1 had reproducible Δt values that remained below the gel specific cut-off threshold between days 36-47, while V5-2 had Δt values that remained below the gel specific cut-off threshold between days 34-40 (FIG. 3.16a). This suggests that all 4 vessels reached steady state prior to initiation of the feeding trial.

V9-R1 and V9-R2 DGGE correlation coefficients remained above their gel-defined cut-off thresholds between days 22-38 (FIG. 25a), with the exception of day 36 and 38 which was within 5% of the gel specific threshold, indicating the vessels shared a high degree of similarity and supported the result that steady state was reached prior to initiation of the feeding trial. DGGE correlation coefficients comparing V5-1 and V5-2 on days 34, 36 and 38 ranged from 50.6% to 55.7%similar, below the gel-specific cut-off threshold (Table 3.15). To compensate for the 7 day separation in the initiation of the RS+ and CS+ feeding trials days 40-45 of V5-2 were compared to day 38 of V5-1 resulting in correlation coefficients ranging from 52.3% to 55.6% similar, below the gel-specific cut-off threshold (Table 3.15). Thus, no substantial changes in vessel similarity occurred between the twin-vessels on days 40-45 for V5-2. This was expected, as moving window correlation analysis resulted in Δt values that were below the gel specific cut-off threshold for V5-2 during this time period, in turn indicating that the vessel had reached a steady state. V5-1 and V5-2 correlation coefficients remained below the gel-specific cut-off threshold during the period preceding the feeding trials indicating the vessels were not similar. This suggests that although both vessels reached steady state the microbial community compositions differentiated during the course of establishing steady state.

Following the initiation of the feeding trial V9-R1(RS+) and V9-R2(CS+) correlation coefficients dropped on days 38-41 (days 1-4 of the feeding trial) below the of the gel specific cut-off threshold to 52.0%, suggesting that the resistant starch was having a unique impact on community composition relative to the cornstarch control. During the wash-out the correlation coefficients increased to 72.2% similar by day 45 (day 8 of the feeding trial), suggesting in turn that the two communities were becoming more similar and possibly in the process of returning to the pre-treatment community composition (Table 3.16, FIG. 25b). These results were mirrored in the moving window correlation analysis throughout the feeding trial (days 37-45), Δt values for V9-R1(RS+) and V9-R2(CS+) varied but were consistently above the gel specific cut-off threshold indicating the communities were not stable but instead were rapidly changing in response to the starch supplemented media (FIG. 25a). Similar results were observed for donor 5 during the course of the feeding trial V5-1(RS+) and V5-2(CS+); Δt values rose above the gel specific cut off threshold during the first 4 days suggesting that the communities were responding to the additional starch substrates and were no longer at a steady state (FIG. 3.16a). V5-1(RS+) Δt values remained above the gel specific cut off threshold until the final day of analysis (day 48) when it dropped to within 5% of the cut-off threshold demonstrating a trend towards steady state. V5-2(CS+) Δt values were within 5% of the gel specific cut off threshold on days 51-55 (FIG. 3.16a) again indicating a trend back towards steady state. V5-1 and V5-2 correlation coefficients dropped daily, during the first 3 days of the feeding trial, to 26.0% similarity then consistently rose until the end of the feeding trail on day 8 with a final similarity of 54.3% (Table 3.16, FIG. 3.16b). These dramatic changes signify that the starch enriched media (RS+ and CS+) had considerable but different influences on the community structure of the fecal microbiota. Furthermore upon termination of the modified media the communities began to revert to a pre-treatment state.

TABLE 3.15
Correlation coefficients (% SI) comparing chemostat communities
inoculated with feces from donor 5 (V5-1 and V5-2) during the period
prior to the in vitro feeding trial.

| Day | V5-1 vs. V5-2 |
|---|---|
| 34 | 55.7 |
| 36 | 50.6 |
| 38 | 53.1 |
| 38 (V5-2)-40 (V5-1) | 54.8 |
| 38 (V5-2)-42 (V5-1) | 54.9 |
| 38 (V5-2)-44 (V5-1) | 55.6 |
| 38 (V5-2)-45 (V5-1) | 52.3 |
| 40 (V5-2)-47 (V5-1) | 60.7 |

TABLE 3.16
Correlation coefficients (% SI) comparing chemostat communities
inoculated with feces from donor 9 (V9-R1 and V9-R2) or donor 5
(V5-1 and V5-2) during the course of the simulated in vitro feeding
trial. Days 1-4: in vitro feeding trial with starch enriched medium,
days 5-8: wash period with basal medium.

| Day | V9-R1 vs. V9-R2 | V5-1 vs. V5-2 |
|---|---|---|
| 1 | 88.1 | 53.9 |
| 2 | 62.8 | 34.0 |
| 3 | 56.9 | 26.0 |
| 4 | 52.0 | 30.1 |
| 5 | 68.2 | 42.1 |
| 6 | 63.8 | 42.8 |
| 7 | 70.1 | 49.0 |
| 8 | 72.2 | 54.3 |

FIG. 23 shows PCA model (panel a) and OPLS-DA model (panel b) of GC/MS data obtained from starch substrate fermentations with chemostat material from donor 9 at 48 h.

Variables are mean-centered and pareto-scaled, model characteristics are as follows: R2X(cum)) 0.691, Q2(cum)) 0.604, and two significant PCs. Figure key: circle Cg102, squares Cgx333, triangles Cg102ae1-Elmore, diamonds Cg102ae1-ref, pentagon Cgx333Su2, stars Cg102wx.

FIG. 24 Flowchart of experimental design of in vitro chemostat feeding utilized in this study

FIG. 25 DGGE analysis of the in vitro feeding trial assessing the effect of a starch enriched media on chemostat communities seeded with feces from donor 9 (V9-R1 and V9-R2). Panel a) Community dynamics calculated using moving window correlation analysis. Panel b) Correlation coefficients (expressed as percentages) comparing the profile similarity of the twin vessels at identical time points.

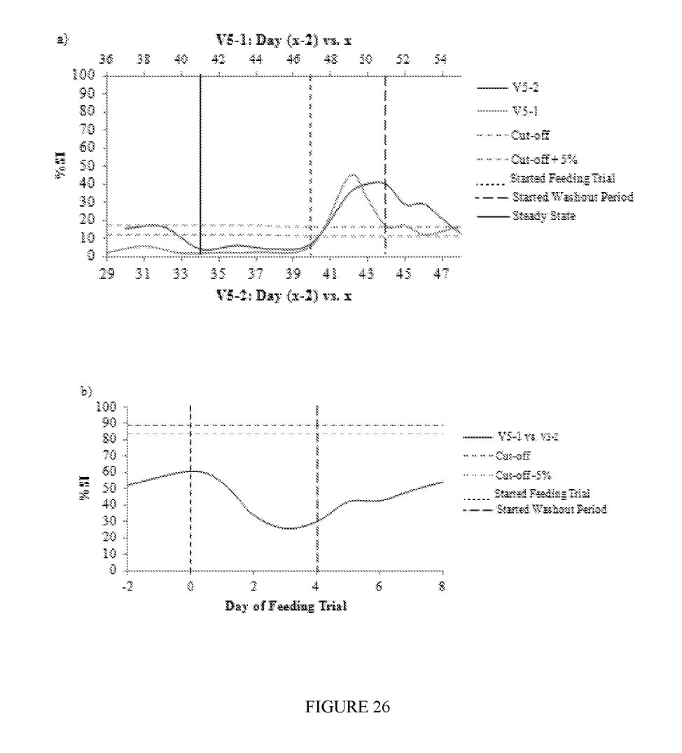

FIG. 26 DGGE analysis of the in vitro feeding trial assessing the effect of a starch enriched media on chemostat communities seeded with feces from donor 5 (V5-1 and V5-2). Panel a) Community dynamics calculated using moving window correlation analysis. Panel b) Correlation coefficients (expressed as percentages) comparing the profile similarity of the twin vessels at identical time points.

### Mauve Alignment

Alignments provided a good visualization of the number of contigs and similarities between species strains. Based on visualization of the alignments, Bifidobacterium adolescentis strains and Lactobacillus casei strains appeared to be very similar. Alignment visualization also showed an early indication that the Ruminococcus obeum strains are more dissimilar than the other five species examined. Difference is alignment could reflect true strain differences, but could also be the result of incorrectly ordered contigs, which appear as genome rearrangements. Alignment figures can be found in FIG. 2.

Functional Comparison using SEED viewer

Table 2 shows SEED viewer functional comparison results. A summary of the functional comparison of pairs of bacterial strains from six different bacterial species based on subsystem annotation; numbers indicate the number of subsystems roles identified to be present in strain A and not strain B, present in strain B and not strain A, or present in both strains and the total number of subsystems roles identified for each species comparison.

TABLE 2
Functional

[symbol]

| Active in | Bifidobacterium | Bifidobacterium | Dorea | Lactobacillus | Ruminococcus | Ruminococcus |
|---|---|---|---|---|---|---|
| [symbol] | [symbol] | [symbol] | [symbol] | [symbol] | [symbol] | [symbol] |
| A not [symbol] | 3 | 14 | 8 | 0 | 3 | 125 |
| B not [symbol] | 3 | 5 | 17 | 1 | 2 | 122 |
| A & [symbol] | 118 | 123 | 123 | 170 | 142 | 126 |
| Tota [symbol] | 119 | 125 | 126 | 170 | 142 | 150 |

[symbol]

indicates data missing or illegible when filed

Functional comparison of the strain pairs for the six bacterial species with two different strains revealed comparatively: very high functional redundancy in three species, high functional redundancy in two species and low functional redundancy in one species. The highest level of functional redundancy using a subsystem-based method of comparison was seen in the comparison of the Lactobacillus casei pairs. The only difference in functional subsystems was identified to be present in strain B and not strain A and involved lactose and galactose uptake (Table 3). The lowest level of redundancy was seen in the comparison of the Ruminococcus obeum strain pairs where 247 differences in functional subsystem roles were identified over a broad range of subsystems and categories. Comparison of both Ruminococcus torques and Bifidobacterium adolescentis strain pairs revealed only five and six differences between strains respectively, a comparatively very high level of redundancy (Table 3). The Bifidobacterium longum comparison of strain pairs showed slightly less redundancy with 19 differences in functional subsystem roles between strain A and strain B, 14 of which were present in Bifidobacterium longum strain A not B and only 5 of which were present in stain B not A. The comparison of Dorea longicatena strain pairs revealed 8 subsystem roles present in strain A not B and 17 subsystems present in strain B not A. A full list of differences in the comparison of functional subsystems for the Bifidobacterium longum and Dorea longicatena strain pairs is available in Table 8.

Table 8 shows a summary of SEED viewer functional comparisons. (A) shows Bifidbacterium longum. (B) Dorea longicatena. A summary of the subsystem based functional differences between strains A and B for Bifidbacterium longum and Dorea longicatena showing the category, subcategory, subsystem, and roles identified. The sections indicated on the row entitled ‘Phages, Prophages, Transposable Elements and Plasmids’ indicate differences related to phage elements.

Table 3 shows a summary of SEED viewer functional comparison. A summary of the subsystem based functional differences between strains A and B for Lactobacillus casei, Bifidobacterium adolescentis, and Ruminococcus torques showing the category, subcategory, subsystem and roles identified. Sections highlighted in grey indicate differences related to phage elements.

A key element to note is the large number of phage-related proteins and roles related to phages present in the comparisons (highlighted in grey text in Table 3 and Table 8). Phage related proteins were present in one strain but not the other for Bifidobacterium longum and Dorea longicatena and were present, but with different roles, in both strains of Bifidobacterium adolescentis and Ruminococcus obeum. These elements could help to explain the differences between these strain pairs. If one strain was infected with a phage while another remained unaffected, or strains were infected by different phages, this could cause the some of the differences in genes and functionality reported in this analysis. This is an excellent explanation of the strain divergence since phages are key horizontal gene transfer (HGT) mediators and an important pathway for gene introduction into the human gut microbiome.

Sequence Comparison using SEED viewer

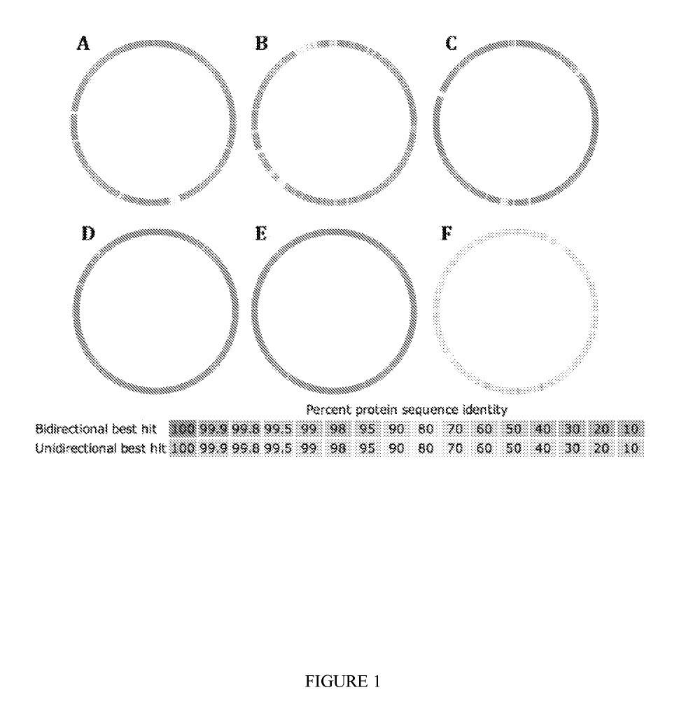

The sequence comparison for the strain pairs of the bacterial species for which two strains had been included in the original RePOOPulate ecosystem revealed similar results to the functional comparison. Five of the six species examined showed high to very high redundancy in their protein sequences. Comparison of the strain pairs for Bifidobacterium adolescentis, Bifidobacterium longum, Dorea longicatena, Lactobacillus casei and Ruminococcus torques all showed an average percent protein sequence identity of 95% or greater (see Table 7). The Ruminococcus obeum strain comparison by contrast had a much lower average percent protein sequence identity of between 45 and 62%, dependent upon whether or not hypothetical proteins were included in the comparison and which strain was used as the reference strain. The differences between the protein sequences can be clearly visualized in FIG. 1, which shows the percent protein sequence identity of strain B for each of the six species when strain A of the same species is used as a reference. The first five species are clearly in the 90% or greater range for the majority of the identified protein sequences, whereas the Ruminococcus obeum strains appear closer to the 50-60% range.

Table 7 shows a summary of SEED viewer sequence comparisons of pairs of bacterial strains from six different bacterial species based on percent protein sequence identity; numbers in brackets indicate comparisons with hypothetical proteins removed. Tables include the total number of proteins identified, the number of bi-directional and uni-directional hits, the total number of proteins with no hits (0%), the total number of proteins with perfect sequence match (100%), the number of proteins with high protein sequence identity (95%-99%), the number of proteins with low protein sequence identity (50% or less, not including those with no hits) and the average percent protein sequence identity. (A) summarizes the sequence comparisons with strain A as a reference strain. (B) summarizes the sequence comparisons with strain B as a reference strain.

FIGS. 1A and 1B show SEED viewer sequence comparison figures for strain pairs. Diagrams show comparison between strain A as a reference sequence and strain B. A) Bifidobacterium adolescentis sequence comparison of strain A to strain B. B) Bifidobacterium longum sequence comparison of strain A to strain B. C) Dorea longicatena sequence comparison of strain A to strain B. D) Lactobacillus casei sequence comparison of strain A to strain B. E) Ruminococcus torques sequence comparison of strain A to strain B. F) Ruminococcus obeum sequence comparison of strain A to strain B.

The linear models that were fitted for the comparison of the average percent protein identity to genomes size and number of contigs indicated that both of these factors could have confounded the results for the SEED sequence comparison to some level. The linear model for the comparison of genome size to average percent protein sequence identity had a p-value of 0.006 indicating a significant linear relationship. The linear relationship between the number of contigs and the average percent protein sequence identity was also significant with a p-value of 0.016. Scatterplots depicting these relationships can be found in FIG. 3.

FIG. 3 shows scatter plots for comparison using R. Plots were created in R using variations of the pseudo-code given below:

Pseudo-code for Linear Models

setwd(“/Users/folder/”)

Table<-read,table(file [symbol] “table.csv”,sep [symbol] “,”,header [symbol] PROG)

LM1<-lm(PercentProteinID [symbol] GenomeSize,data [symbol] Table

summary(LM1)

plot(Table$GenomeSize,Table$PercentProteinID)

abline(LM1)

FIG. 3A shows a scatter plot of Genome Size versus Average Percent Protein Sequence Identity for the 12 bacterial genomes analyzed in Part I, with line showing the linear correlation between the two. Linear model has a p-value of 0.006144. FIG. 3B shows a scatter plot for the Number of Contigs versus Average Percent Protein Sequence Identity for the 12 bacterial genomes analyzed in Part I, with line showing the linear correlation between the two. Linear model has a p-value of 0.01629. FIG. 3C shows a scatter plot for Genome Size versus Number of Contigs for all 33 bacterial genomes. An outlier is Eubacterium rectale 18FAA, which appears to have had an error in sequencing.

KEGG Pathway Analysis

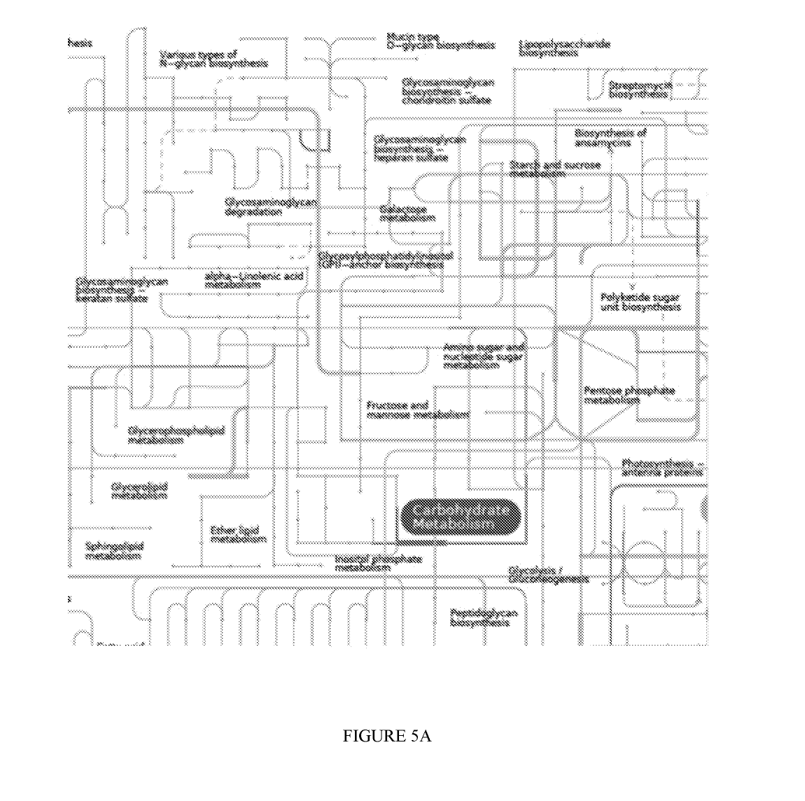

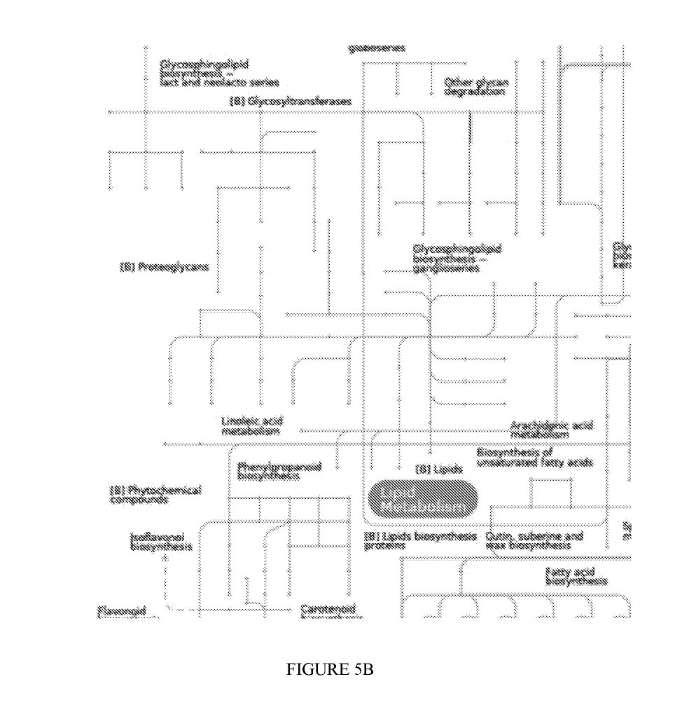

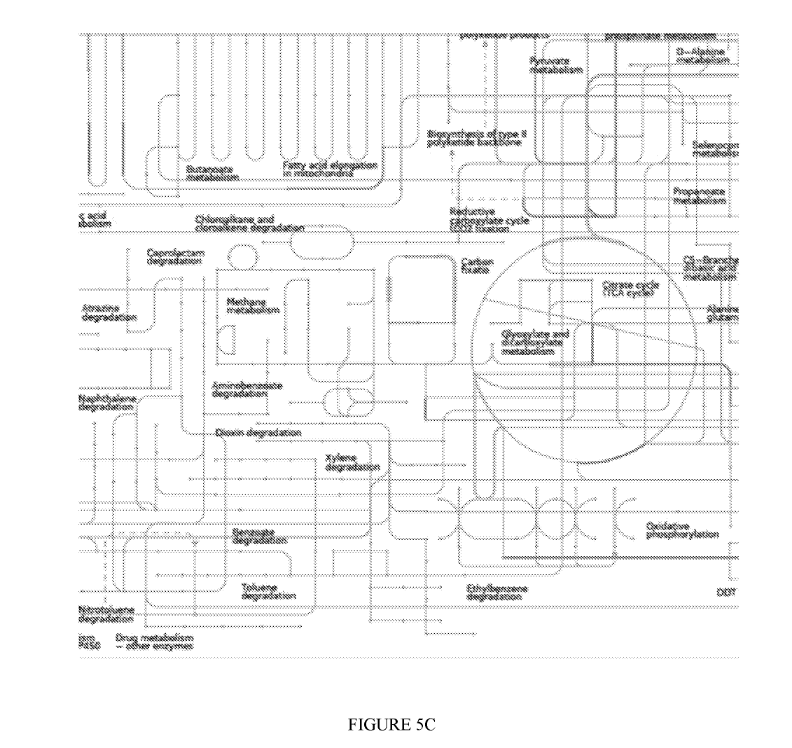

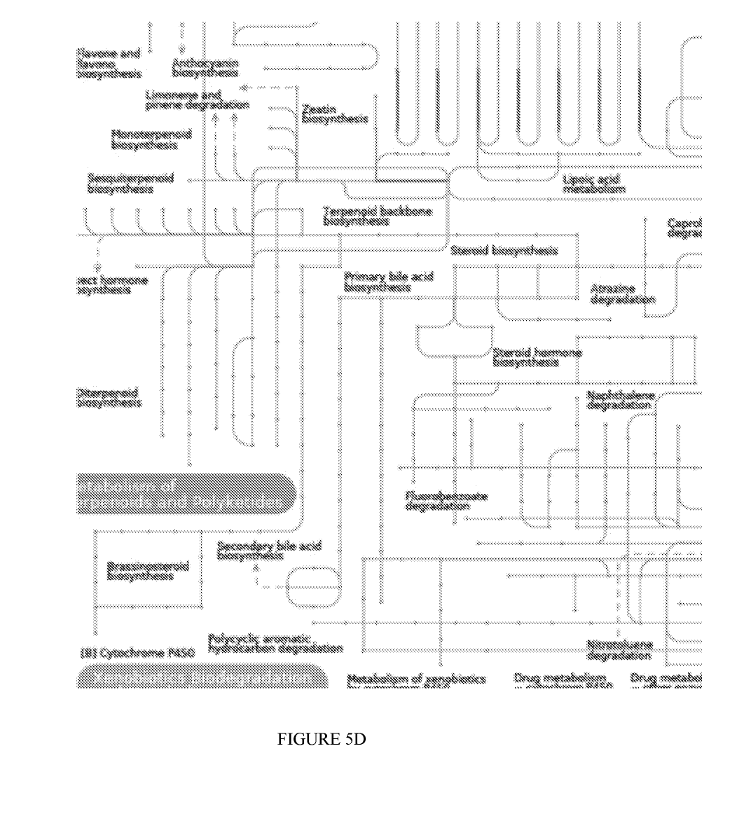

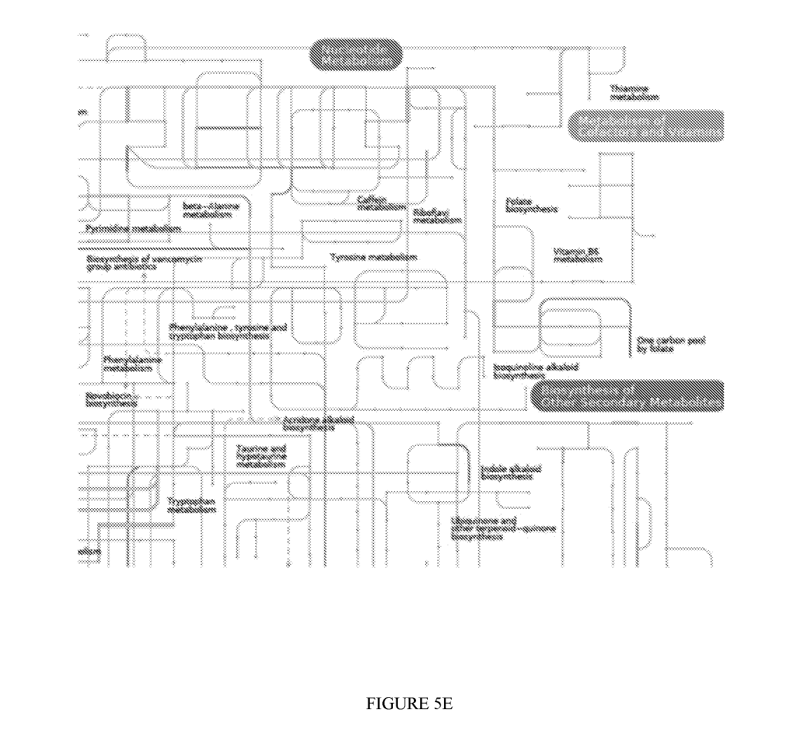

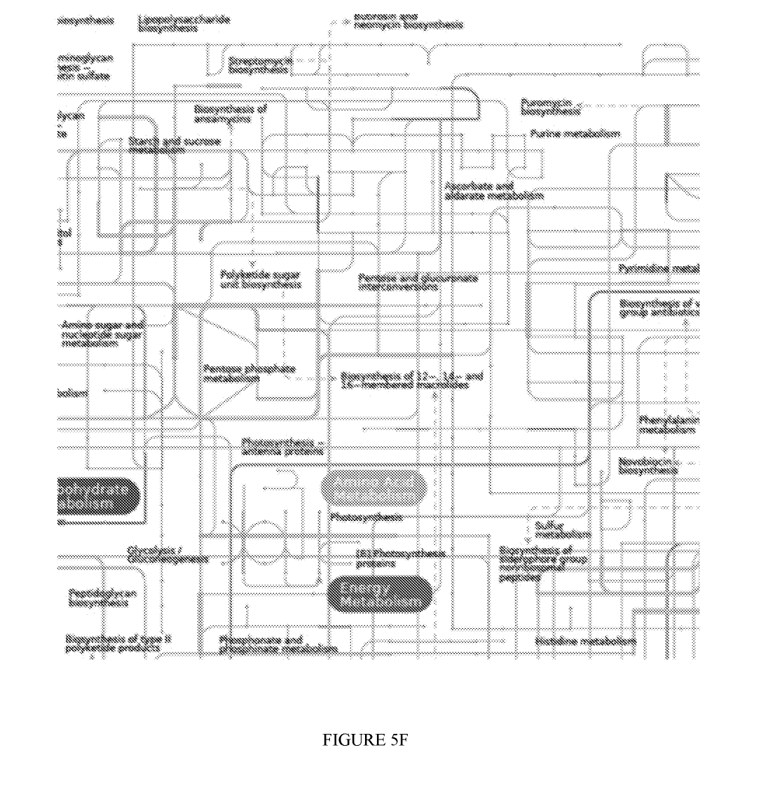

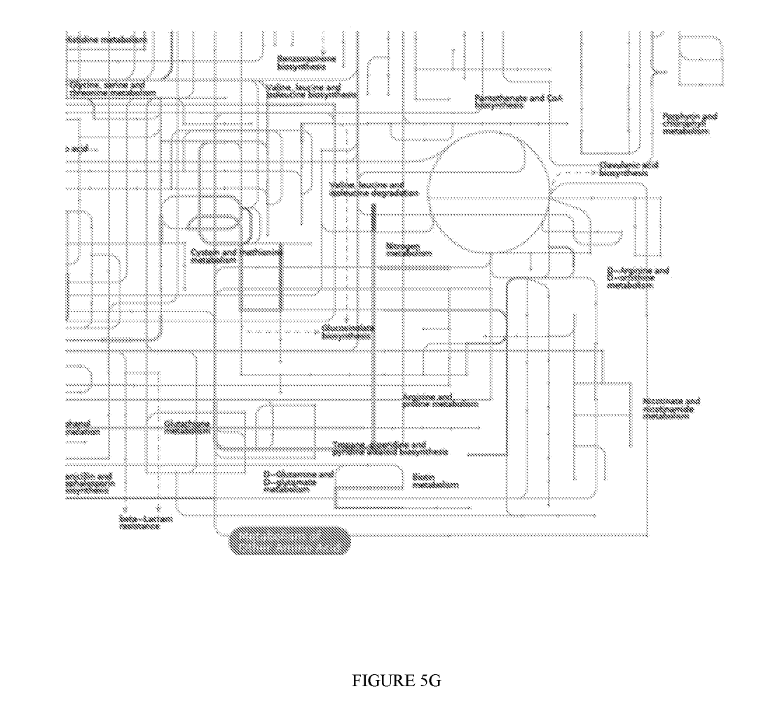

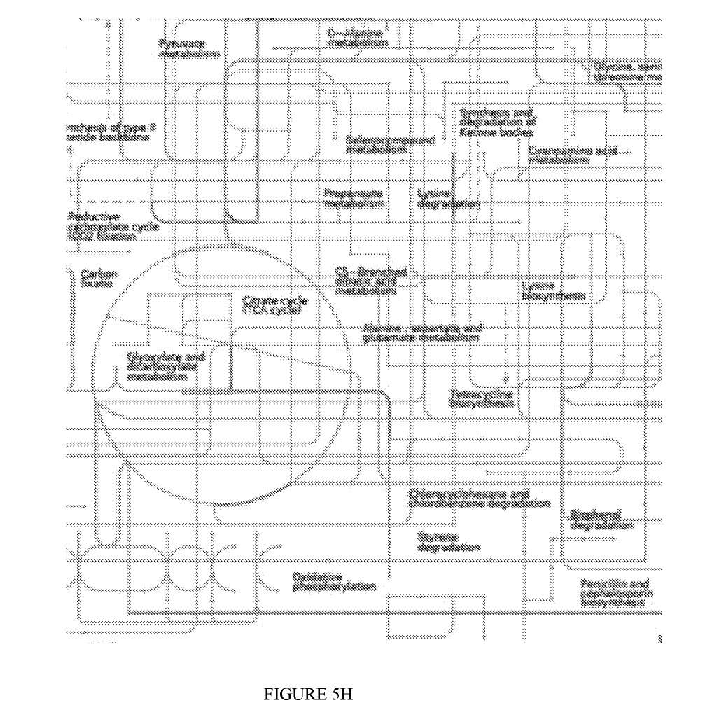

The KEGG pathway results confirmed the results of the functional and sequence comparisons using the SEED viewer. Comparison of KEGG Orthology for Bifidobacterium adolescentis, after ID matching to the internal iPath2.0 list and conflict resolution, revealed only three key differences in pathways that were present in strain B and not present in strain A. The Bifidobacterium longum KEGG comparison initially revealed 40 differences in KO IDS between strain A and B, however after matching and conflict resolution 5 KO IDs unique to strain A and 3 KO IDs unique to strain B, as well as 4 KO IDs with a higher number of replicates in strain A and 2 KO IDs with a higher number of replicates in strain B were found. The Lactobacillus casei KEGG pathway comparison revealed only one difference, a KO ID that was unique to strain B. This is consistent with the high level of redundancy between the Lactobacillus casei strains seen throughout this study. The Dorea longicatena comparison revealed 2 unique KO IDs for strain A and 6 unique KO IDs for strain B. The Ruminococcus torques KEGG comparison found only 2 unique KO IDs for each strain. A full list of the differences in KEGG Orthology assignments for these five species, and the pathway elements that they map to can be found in Table 9. The comparison of Ruminococcus obeum strains based on KEGG Pathway analysis revealed much the same results as the previous sections. The comparison found 43 unique IDs for strain A and 32 unique IDs for strain B, as well as 5 IDs with greater replication in strain A and 3 IDs with greater replication in strain B (FIG. 5). This is consistent with the low levels of redundancy seen in the SEED viewer comparison, indicating the necessity of both Ruminococcus obeum strains. These results, when combined with the results from the SEED viewer comparisons, indicate that strain A for Bifidobacterium adolescentis, Lactobacillus casei, and Dorea longicatena, as well as strain B for Bifidobacterium longum and Ruminococcus torques appear to be functionally redundant and could be removed from the ecosystem without causing an ecological imbalance.

FIGS. 5A-B shows KEGG pathway maps for comparing Ruminococcus obeum. FIG. 5A shows the metabolic pathway map. FIG. 5B shows the regulatory pathway map. KEGG pathway maps were generated using ipath2.0 for the comparison of Ruminococcus obeum strain A to strain B. Green lines represent shared pathways, red lines represent pathways unique to strain A or with greater repetition in strain A, blue lines represent pathways unique to strain B or with greater prepetition in strain B. Line weights are determined by number of repeats of KO IDs.

Table 9 shows a summary of the differences in KEGG pathways for five of the species compared in Part I. Table 9 includes the KO ID, the map(s) name (including biosynthesis of secondary metabolites, Sec. Biosynth.) and the specific pathway elements that are unique to one strain. Sections in blue indicate KO IDs and elements that are not unique to one strain but have a higher number of replicates in the strain indicated.

Part II: Redundancy within the RePOOPulate Ecosystem

Methods

Redundancy within the RePOOPulate ecosystem was examined in much the same way as the KEGG pathway comparison described above, but on a larger scale. KAAS (KEGG Automatic Annotation Server) was used to provide functional annotation of the genes in the draft genomes not included in Part I (21 further genomes). The lists of KO assignments (KO IDs) for each genome were downloaded and compared in a table in Microsoft Excel. A list of KO IDs found for all thirty-three species within the original RePOOPulate ecosystem, as well as a list of counts of the number of times a KO ID was found within the entire ecosystem was created from the Microsoft Excel table. These lists were then used to create a final list of KEGG IDs with weights that matched the number of replicates of a KEGG orthology assignment (KO ID). The list of KO IDs was then imported into the program iPath2.0: interactive pathway explorer and matched to the internal list used for by iPath2.0 before mapping; this removed several KO IDs from the list. This final matched list for all thirty-three species was used in Part III.

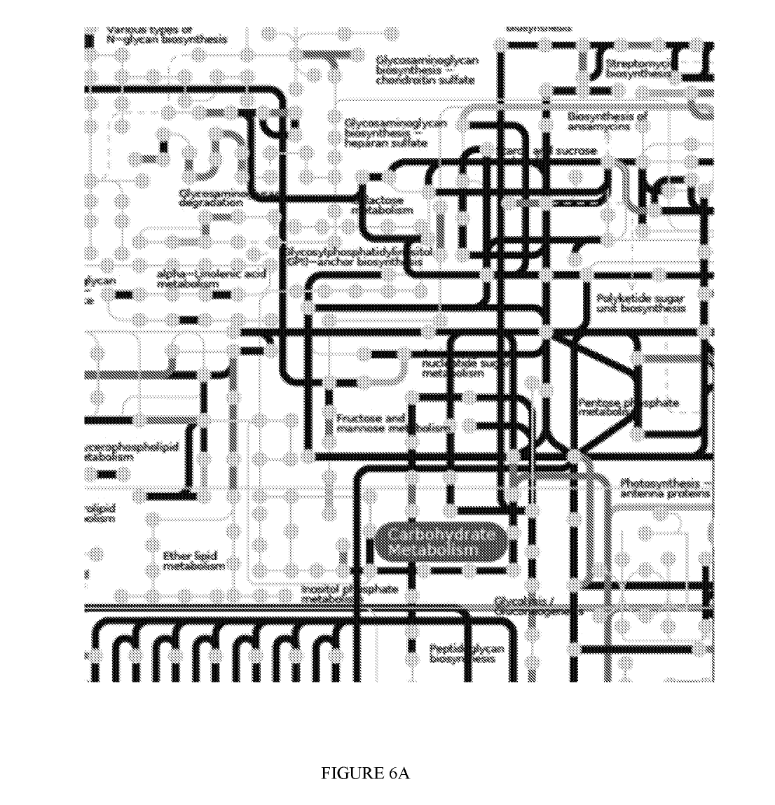

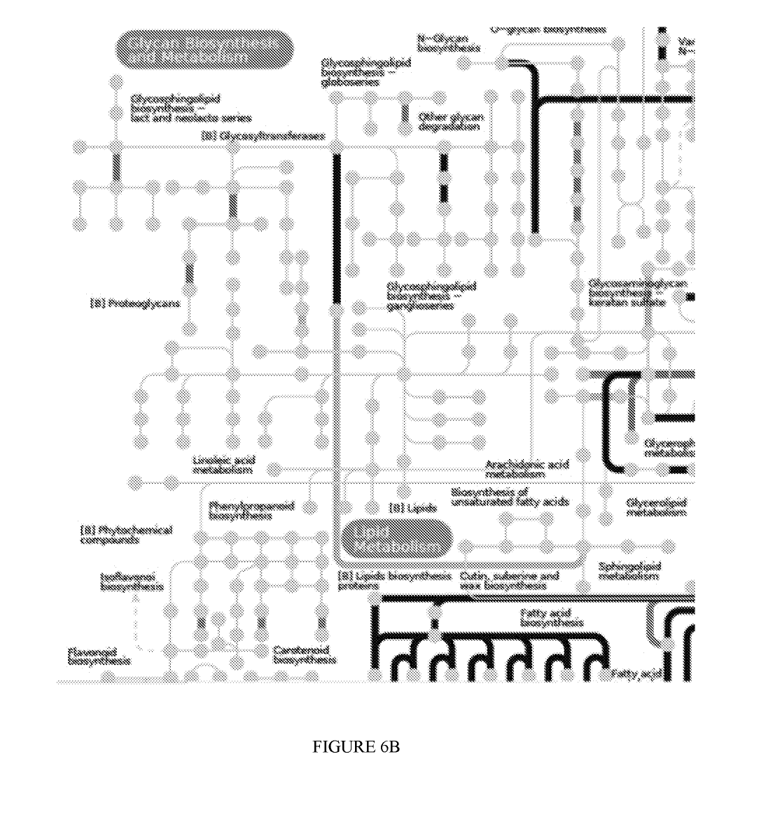

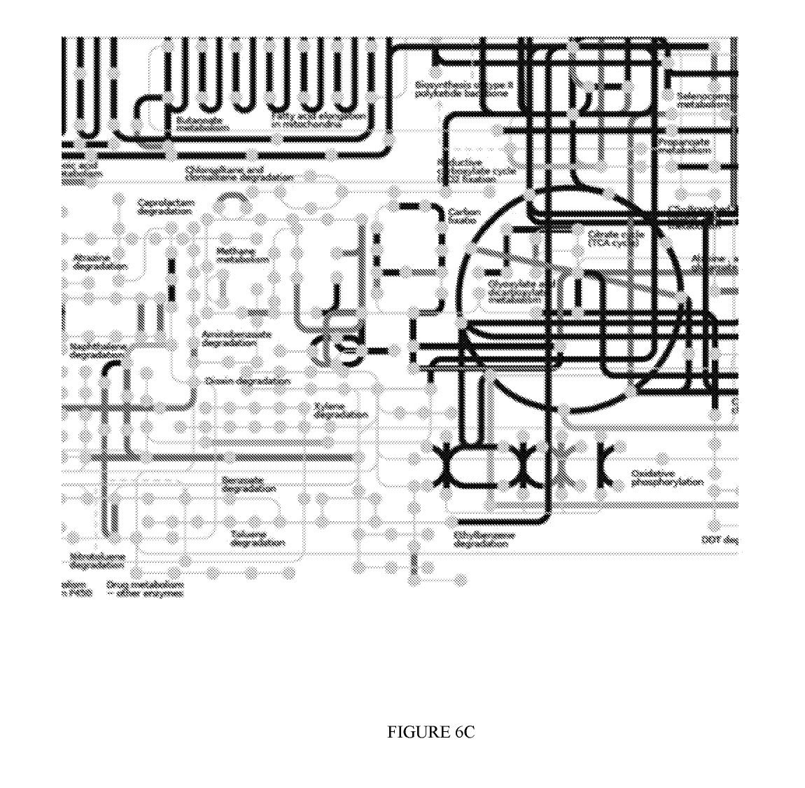

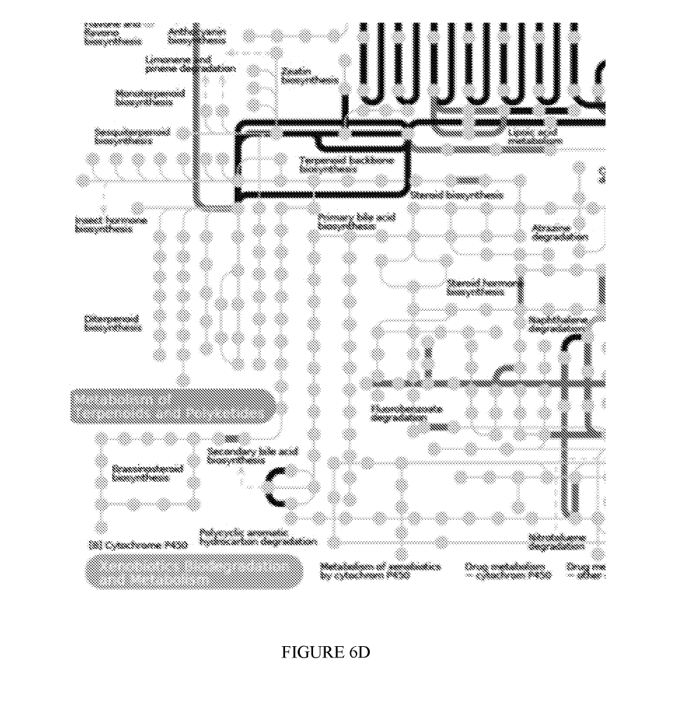

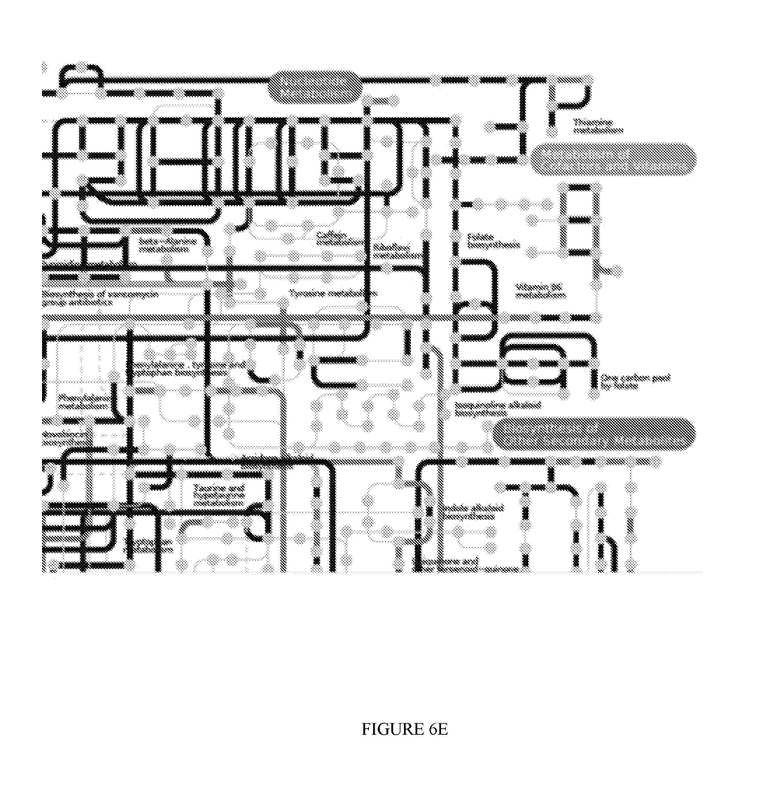

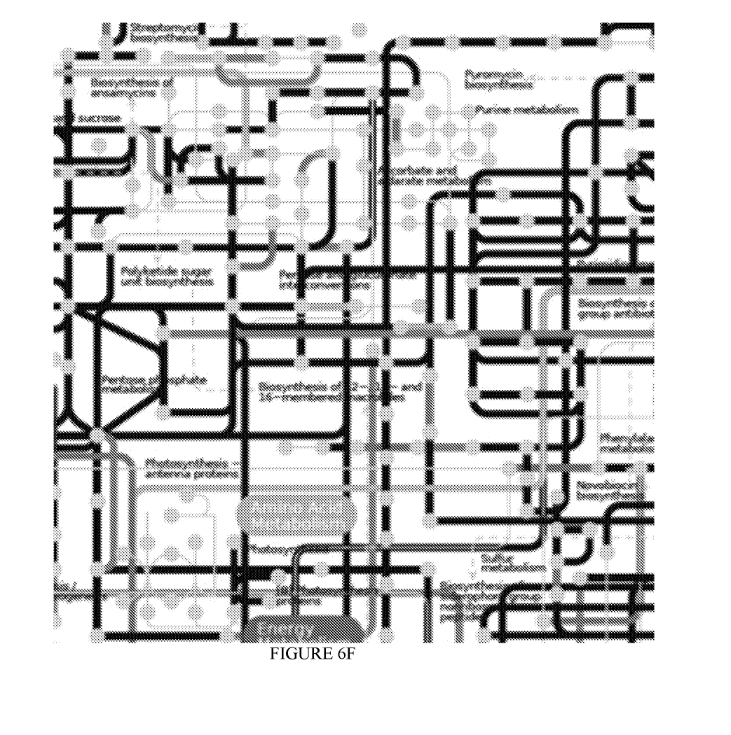

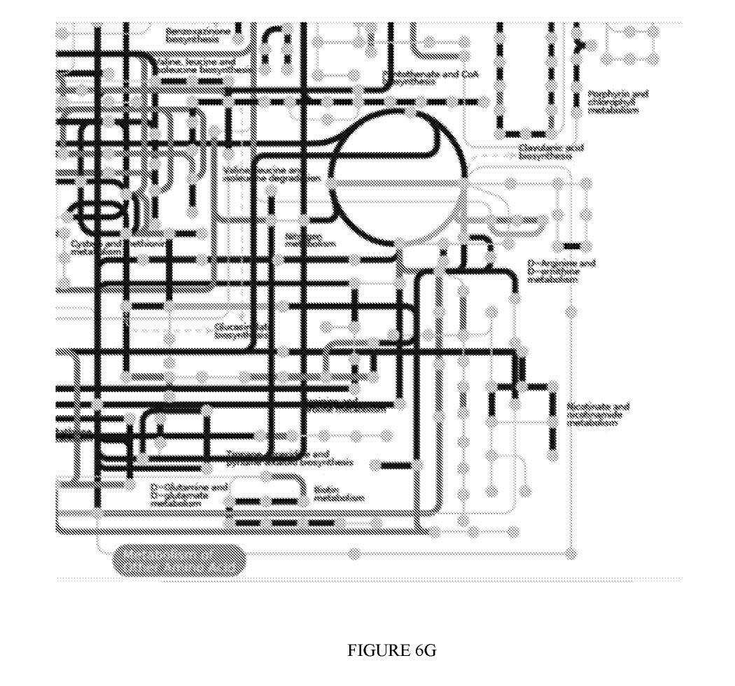

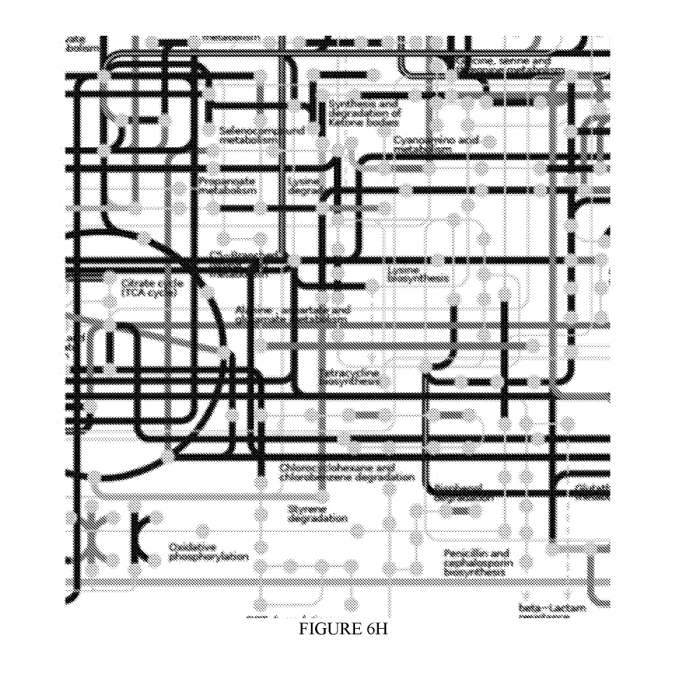

An updated list was next created following the removal of the eight species strains found to be redundant in Part I of this study (Table 4). The second list included only twenty-five different bacteria. A list of matched KO IDs for this smaller ecosystem was created, as well as lists of KO IDs specific to a single species, shared by two species, shared by three species, shared by four species and shared by five or more species. A list of counts of the number of replicates for each KO ID was also created. The lists of KO IDs shared by 1, 2, 3, 4, and 5 or more species were each color coded (purple, blue, green, red and black respectively) and imported into iPath2.0. Conflicts between colors were resolved as the color of the highest number of species it conflicted with, i.e., if a pathway had a conflict between red (4 species) and blue (2 species) it would resolved as red. The final metabolic pathway map was examined (FIG. 6) and counts of the number of nodes shared between each color were counted. Nodes in the map correspond to various chemical compounds and edges represent series of enzymatic reactions or protein complexes. Maps were also created for 1, 2, 3 and 4 species individually to obtain the number of pathway elements (edges) that their KO IDs mapped to (Table 10).

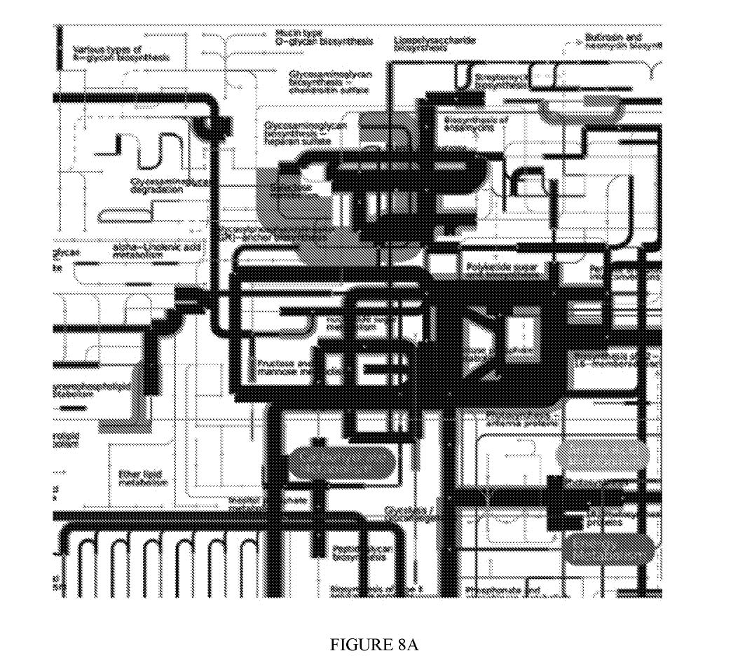

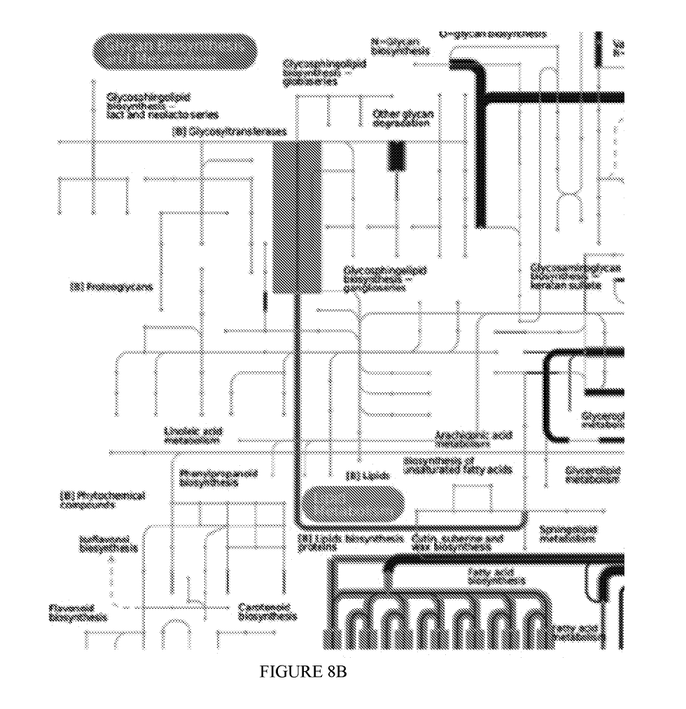

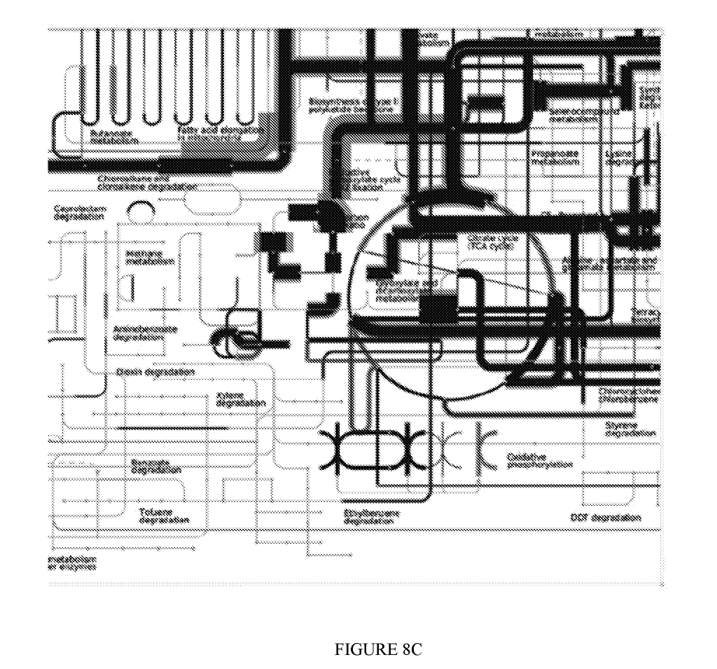

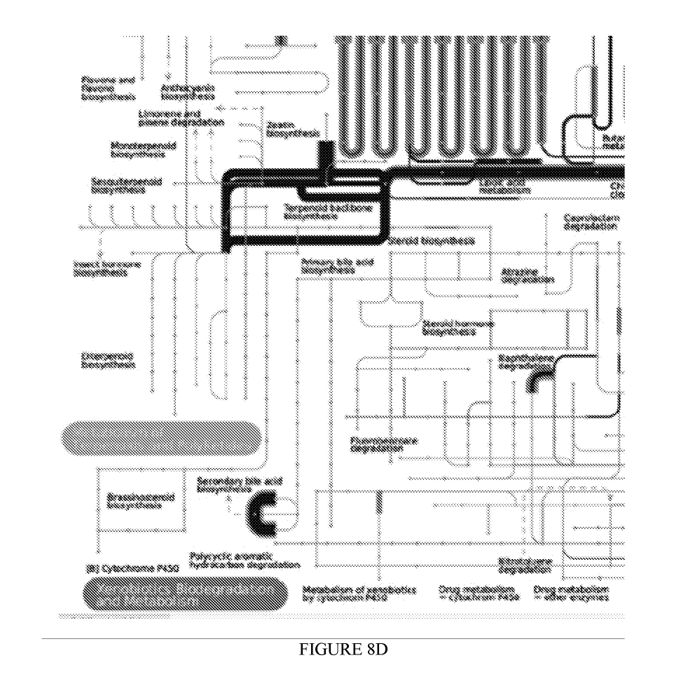

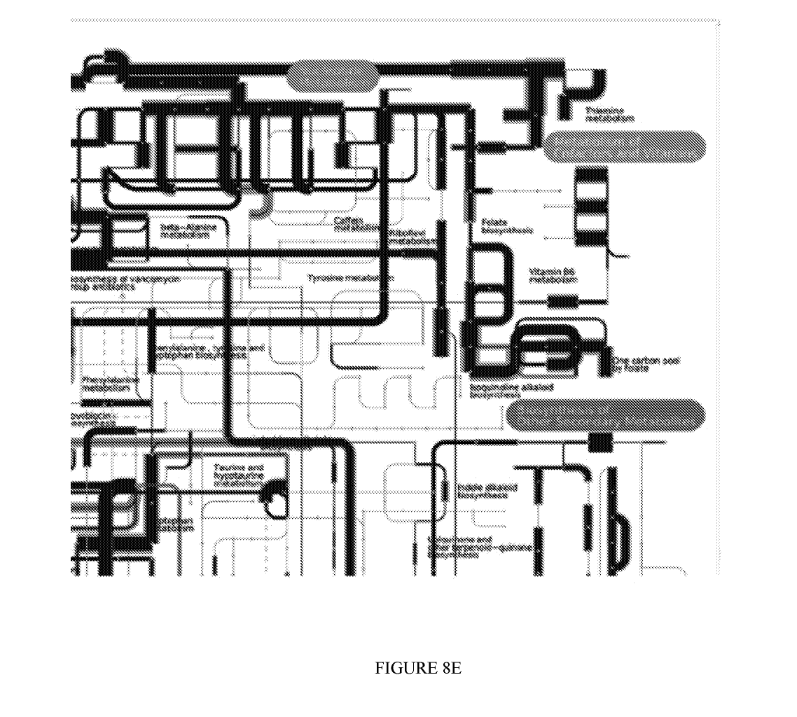

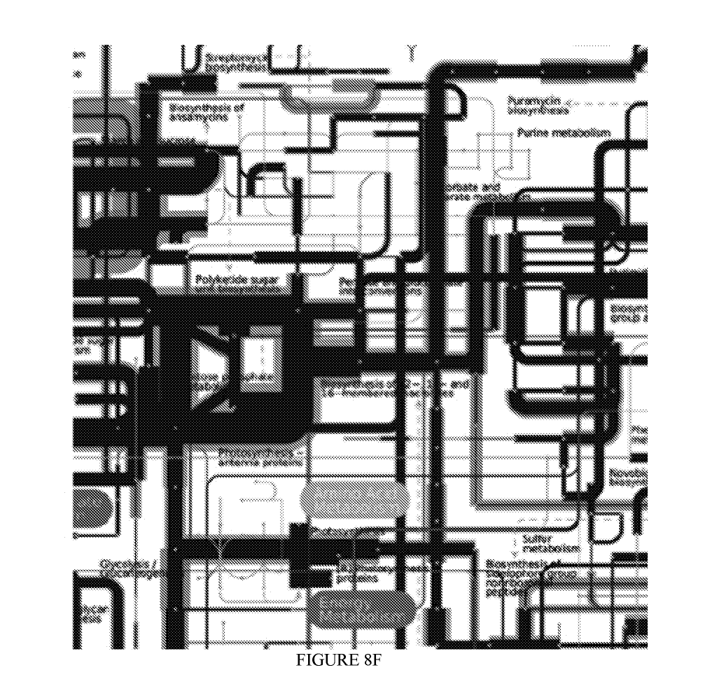

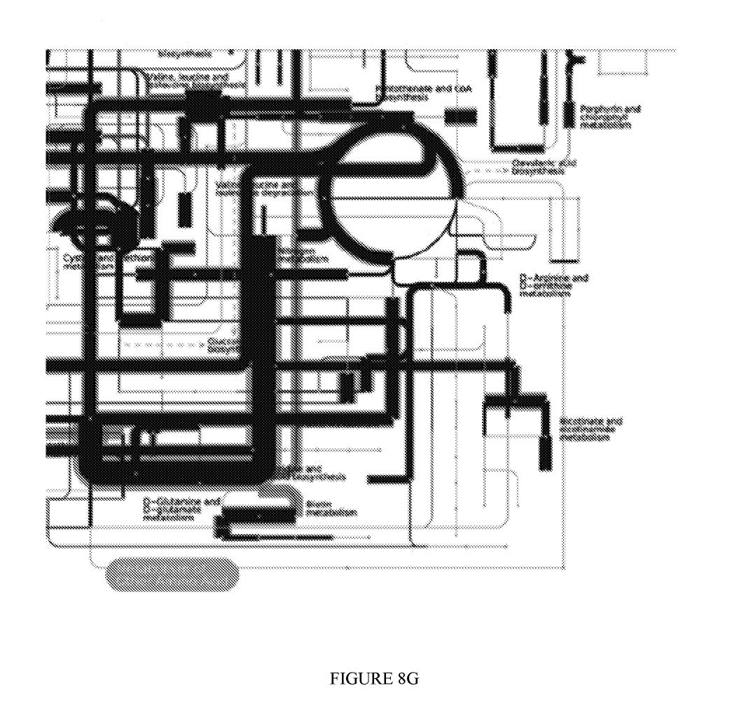

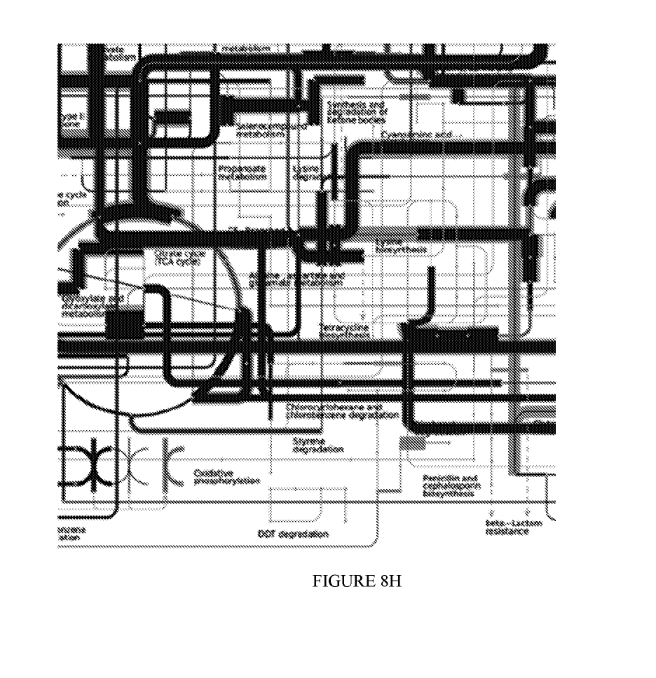

Table 10 shows element counts for ipath2.0 KEGG comparison pathways shared by one, two, three or four species. A summary of the results for the comparison of the RePOOPulate species after redundant strains for Part A were removed (includes 25 species), looking at the pathways shared by one, two, three and four species. Includes the number of pathway elements selected on each of the tree maps, and the counts for the number of unique nodes and shared nodes for the metabolic map (FIG. 8). Unique nodes were counted if the nodes were only part of a pathway that include the number of species shown, nodes shared by greater than four (>4) species were counted if one or more colored lines and a black line shared a node, nodes shared by 1/2/3/4 species were counted where two different colored lines shared a node, i.e. blue (two species) and green (three species).

FIG. 6 shows the metabolic pathway map for ipath 2.0 KEGG comparison of pathways shared by one, two, three or four species. Full metabolic pathway map for the comparison of the RePOOPulate species after redundant strains for Part I were removed (includes 25 species), showing metabolic pathways shared by one, two, three, or four species. Purple lines correspond to unique pathways shared by a single species, blue lines correspond to metabolic pathways shared by two species, green lines correspond to pathways shared by three species, red lines correspond to pathways shared by four species and black lines are all other pathways within the system (>4 species). Line weights were chosen for ease of visualization and do not reflect the number of copies of the KEGG orthology IDs.

The list of KO IDs specific to a single species revealed that only twenty-two of the twenty-five included bacteria had unique KO IDs, the three apparently redundant strains included: Dorea longicatena 42FAA, Eubacterium rectale 29FAA, and Eubacterium ventriosum 47FAA. These three species were removed and the replicate counts were updated to reflect the removal of these three species. The list of matched KO IDs specific to a single species was next used to manually create a color key, which matches a unique color to each species that had KO IDs not shared by any other species. The color key was then used to create a list of KO IDs and matching colors, black for shared KO IDs and a different color for each species with unique KO IDs. This list was imported in iPath2.0 and used to create a custom map. This created a list of color conflicts. Any color conflicts were resolved as black, since this meant the pathway was not unique to a single bacteria. The exception was a conflict with the only unique KO ID for Bifidobacterium longum (K00129), further investigation found that the conflict only affected one of the six pathways that the KO ID mapped to and the conflict was resolved not resolved as black but instead matched to specific color for Bifidobacterium longum.

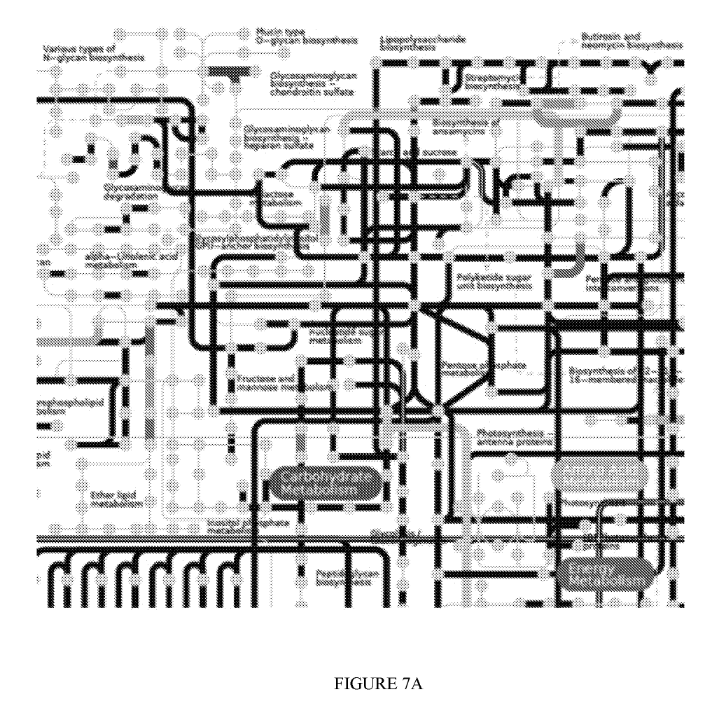

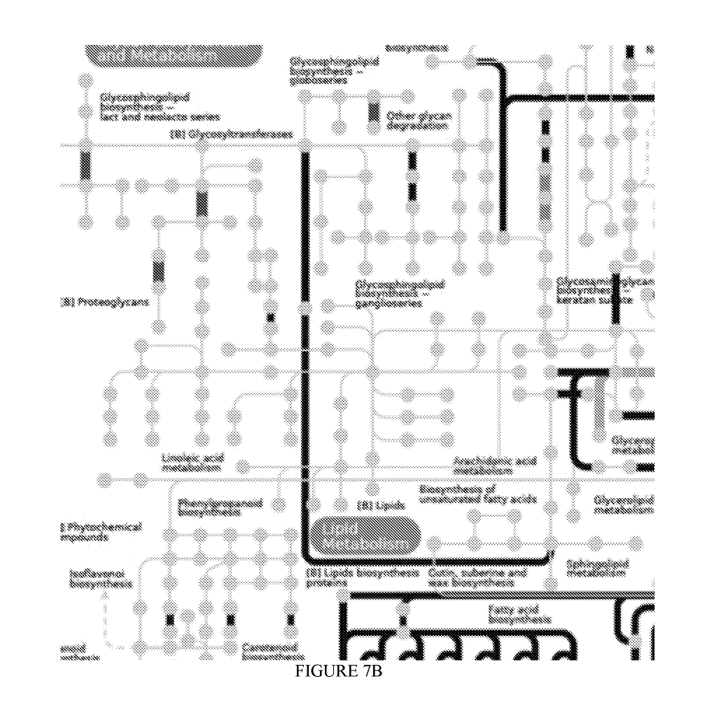

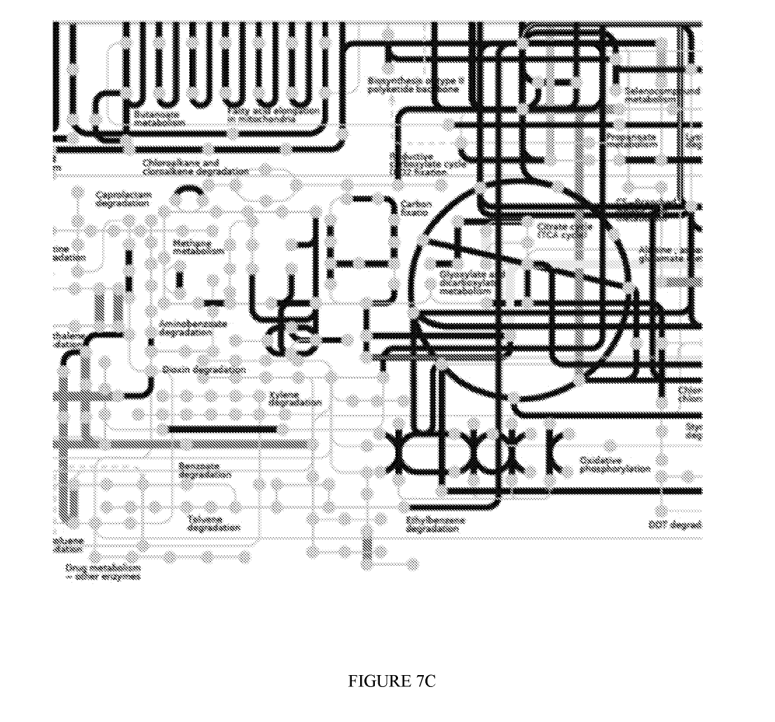

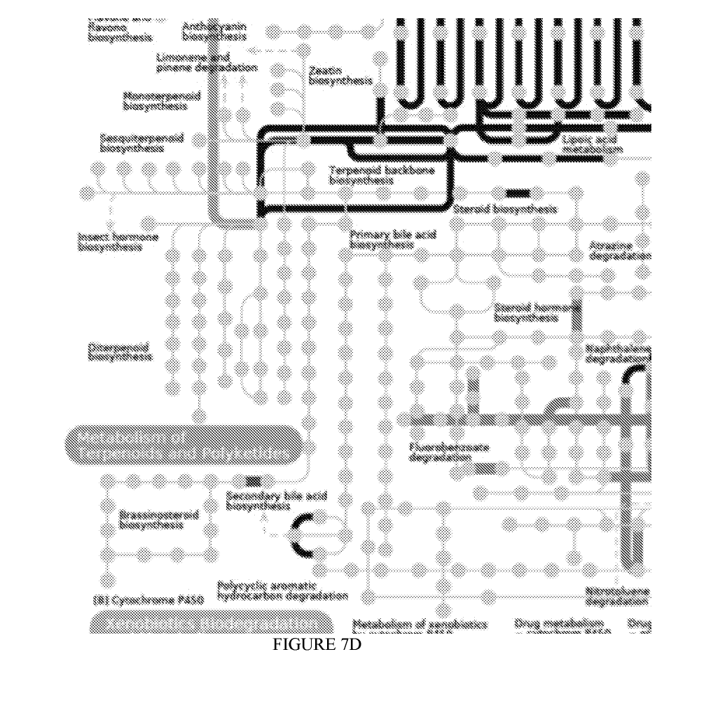

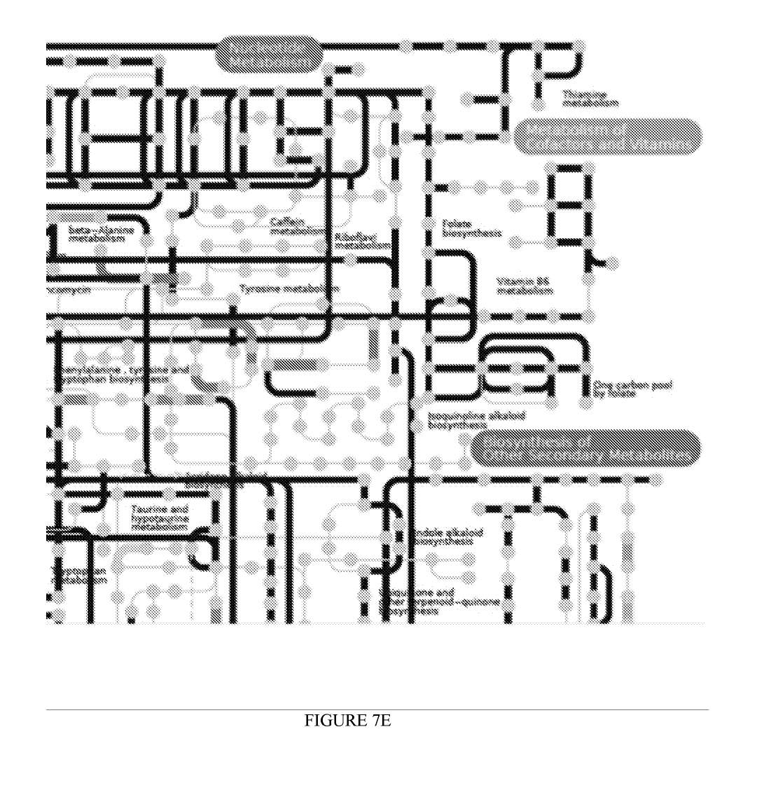

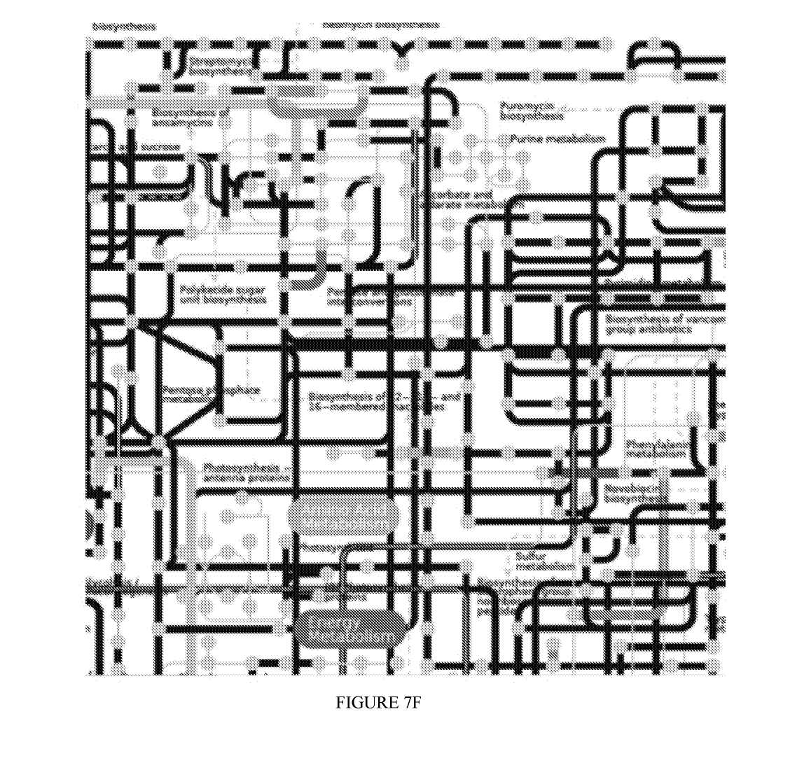

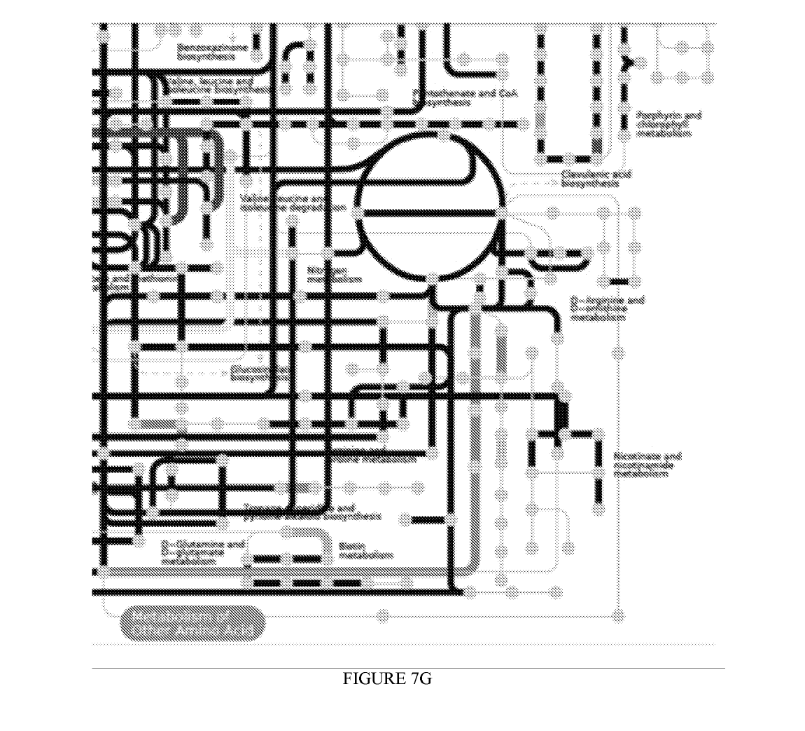

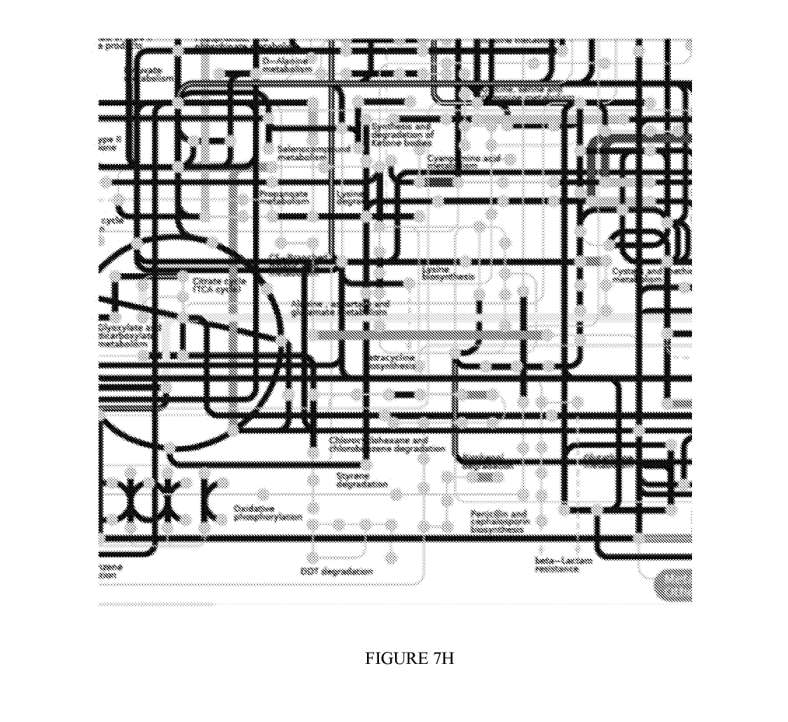

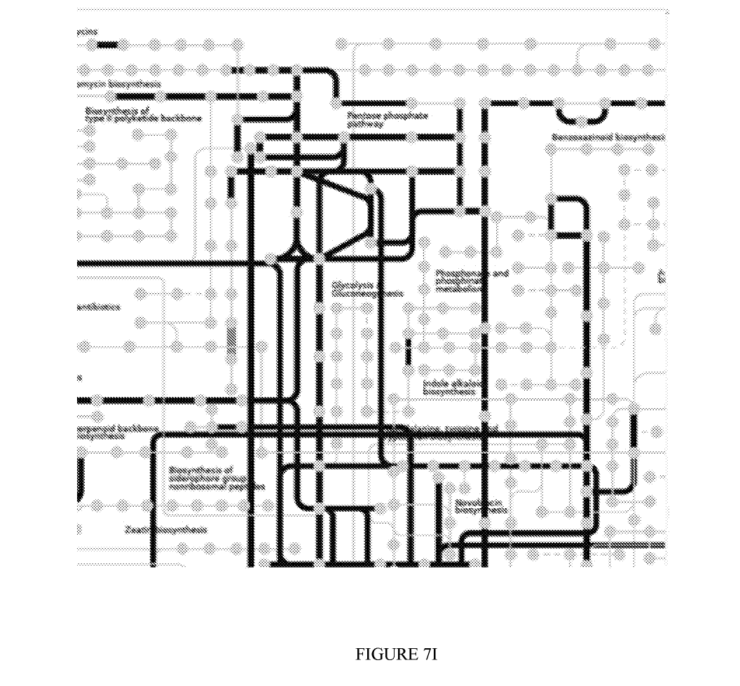

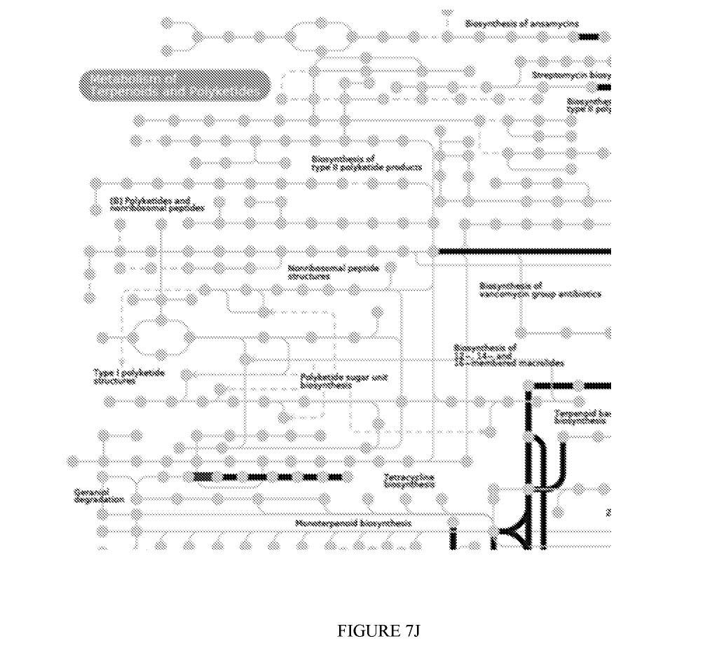

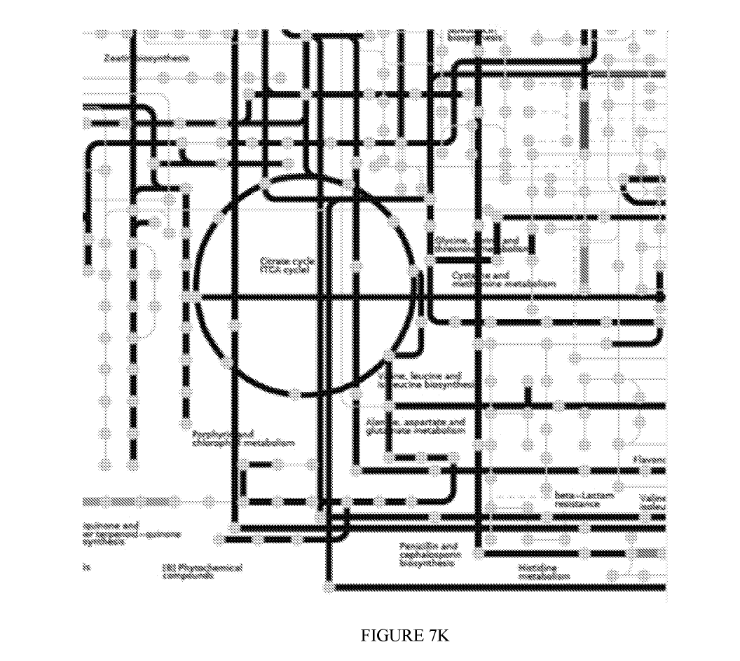

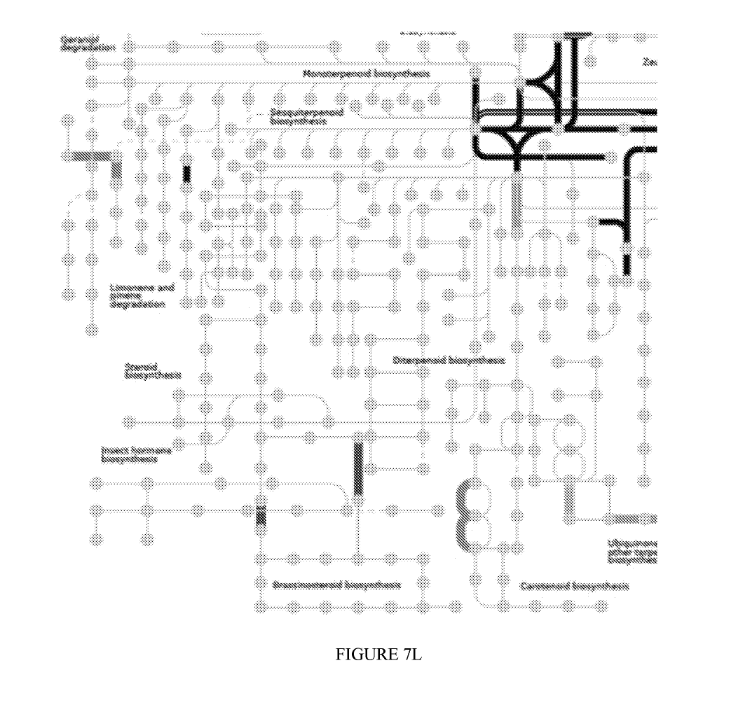

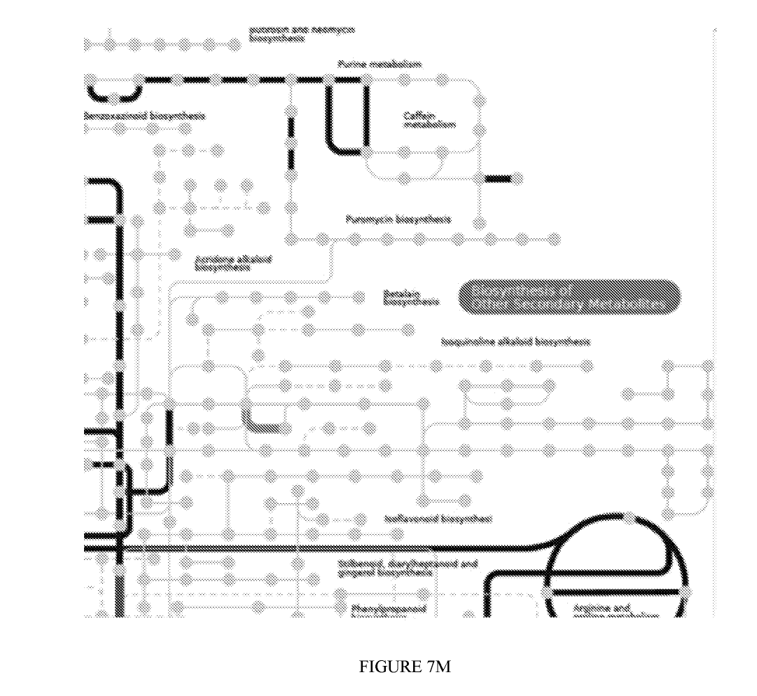

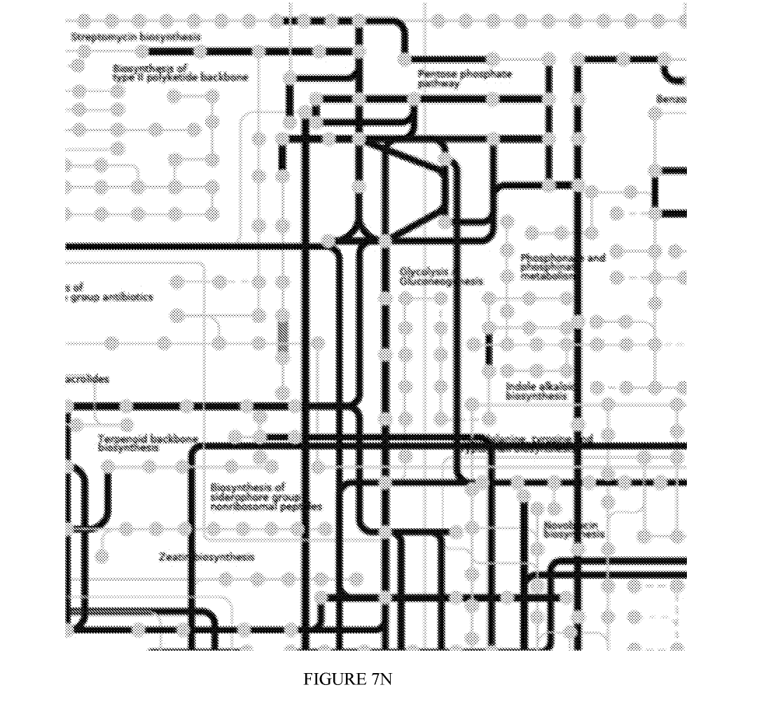

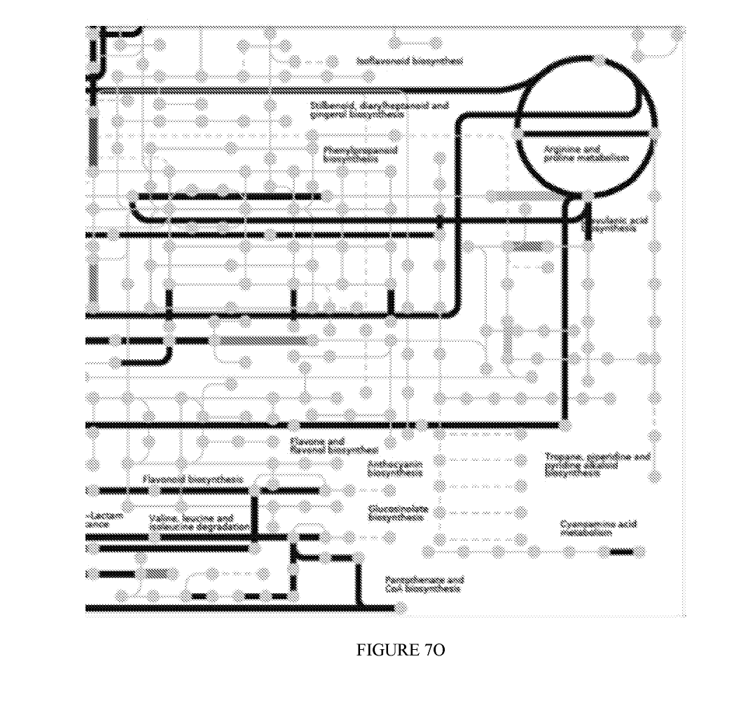

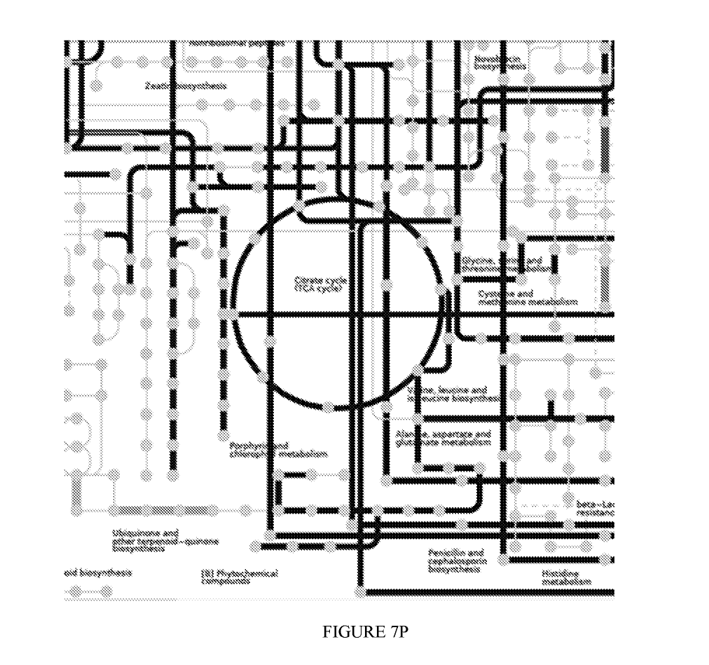

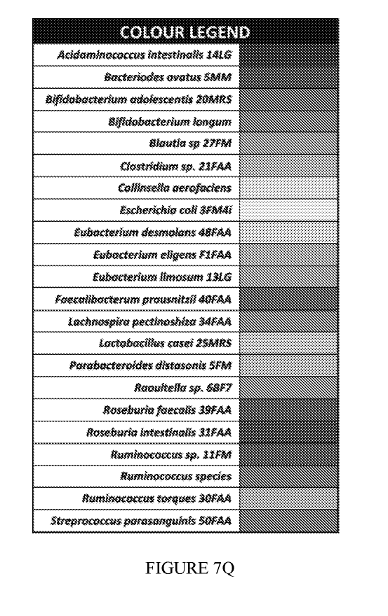

Following conflict resolution a final map was created with black lines for shared pathways and different colored lines for each species with unique KO IDs (FIG. 7). The metabolic and biosynthesis of secondary metabolites maps were analyzed to obtain the number of unique nodes and the highest number of connected nodes. Theses were examined since there are a large number of biochemical and metabolic pathways in bacteria that remain unknown; therefore these element counts may give a better understanding of possible underlying pathways than examining the edges alone (Table 11).

Table 11 shows the element count for ipath2.0 KEGG pathway analysis. A summary of the results for Part II: Redundancy within the RePOOPulate ecosystem including the names of the twenty-two species with unique KO IDs, the number of unique pathway elements that those KO IDs map to for each of the three maps (unique pathways) and a count of the number of unique nodes and the highest number of connected nodes for metabolic and biosynthesis of secondary metabolites maps. Unique nodes were counted if the nodes are part of a unique pathway only and not shared by any other pathways. Numbers in brackets are the number of shared nodes that were also part of a unique pathway. Nodes connected were counted as the highest number of unique nodes connected by unique pathway elements. Numbers in brackets are the highest number of nodes connected by unique pathway elements if the shared nodes that are also part of a unique pathway are included.

FIG. 7 shows the KEGG pathway maps for RePOOPulate population comparison. FIG. 7A shows a full metabolic pathway map for the comparison of 25 species (redundant strains removed) from the original RePOOPulate ecosystem, showing all pathways unique to a single strain. FIG. 7B shows a full regulatory pathway map for the comparison of all 25 species (redundant strains removed) from the original RePOOPulate ecosystem, showing all pathways unique to a single strain. Color legend to the left indicates which color correlates to which species. Line weights were chosen for ease of visualization and do not reflect the number of copies of the KEGG ID.

A final list containing only the unique KO IDs for the twenty-two species with unique KO IDs and matching color codes was used to create maps showing only the unique pathways (FIG. 8). These maps were analyzed to help determine the keystone species and pathways (Table 12). The final list of all KO IDs for the twenty-two species was compared to the list of KO IDs for the original thirty-three species to determine whether any KO IDs had been lost in the process. The list of KO IDs for the final twenty-two species with a list of weights reflecting the number of copies of the KO IDs was used again in Part III of this study. A simple quality check was also performed on the data to see if any obvious errors in the sequencing and genome assembly were evident. Genome size and the number of contigs for all thirty-three genomes were compared using a scatter plot created in R (FIG. 3C). The error in Eubacterium rectale 18FAA, which has been previously noted, was evident and all other genomes appear normal.

Table 12 shows a summary of the unique KEGG pathways of the RePOOPulate ecosystem. Summary of the metabolic and regulatory pathways and the biosynthesis of secondary metabolites for the 22 bacterial species with unique KO IDs after removal of the redundant strains found in Part I. Includes the names of the species with unique KO IDs following matching and conflict resolution with their unique KO IDs and the pathways that they map to. Colors reflect the color legend used for the metabolic and regulatory pathway maps (FIG. 7). KO IDs in red (3) are the unique IDs found only following removal of Dorea longicatena 42FAA, Eubacterium rectale 29FAA, and Eubacterium ventriosum 47FAA in Part II. KO IDs in blue (14) were also found in the Kurokawa et al. data set. Numbers in brackets indicate the number of elements within each of the three maps the KO ID maps to.

FIG. 8 shows the regulatory pathway map for the comparison of twenty-two species from the original RePOOPulate ecosystem (redundant strains removed) showing the regulatory pathways unique to a single strain. Color legend to the left indicates which color correlates to which species. Line weights were chosen for ease of visualization and do not reflect the number of copies of the KO IDs.

Table 4. Summary for the RePOOPulate Bacterial Species. Table includes all thirty-three species included in the original RePOOPulate prototype by name listed on the RAST server. Species are separated into three categories based on the analysis in Part I and II. The twenty-two species found to have unique KEGG pathways after removal of the redundant strains found in Part I are in the first two columns, the eight species strains found to be redundant in Part I of the study and three species found to be redundant in Part II are in the last column. The nine species listed in bold are species with unique KO IDs also present in the Kurokawa et al. data, numbers in brackets indicate the number of KO IDs.

Included in Optimized Ecosystem

| | |
|---|---|
| [symbol] 14LG | Faecalibacterum prausnitzii 40FAA |
| (2) | |
| [symbol] 5MM (2) | Lachnospira pectinoshiza 34FAA |
| [symbol] sp. 21FAA (1) | Bifidobacterium adolescentis 11FAA |
| [symbol] 3FM4i (1) | Bifidobacterium longum |
| [symbol] F1FAA (1) | Blautia sp 27FM |
| [symbol] 13LG (3) | Roseburia faecalis 39FAA |
| [symbol] 25MRS (2) | Roseburia intestinalis 31FAA |
| [symbol] 5FM (1) | Ruminococcus species |
| [symbol] sp. 6BF7 (1) | Ruminococcus sp. 11FM |
| Collinsella aerofaciens | Ruminococcus torques 30FAA |
| Eubacterium desmolans 48FAA | Streprococcus parasanguinis 50FAA |

Removed in Part I
Bifidobacterium adolescentis 11FAA
Bifidobacterium longum 4FM
Dorea longicatena 10FAA
Lactobacillus casei 6MRS
Ruminococcus torques 9FAA
Eubacterium rectale
Eubacterium rectale 6FM
Eubacterium rectale 18FAA
Removed in Part II
Dorea longicatena 42FAA
Eubacterium rectale 29FAA
Eubacterium ventriosum 47FAA

Results

The comparison of the unique and almost unique pathways and nodes, shared by one, two, three or four species or strains, revealed several interesting patterns. A comparison of the pathways shared by two, three and four species was done in order to give an idea of redundancy within the ecosystem that cannot be easily removed (because the pathway is rare overall to the ecosystem, but not unique). The KEGG orthology assignment comparison of the twenty-five species within the bacterial community that remained, after the removal of the redundant species in Part I, revealed three species that did not have unique KO IDs and appear to be further redundancies within the ecosystem (Dorea longicatena 42FAA, Eubacterium rectale 29FAA, and Eubacterium ventriosum 47FAA). When the almost unique pathways for these three species were examined there was also only a low number of almost unique pathways. When comparing KO IDs shared by two, three and four species respectively, Eubacterium rectale 29FAA had 3, 1 and 3 shared KO IDs, Dorea longicatena 42FAA had 3, 5 and 3 shared KO IDs and Eubacterium ventriosum 47FAA had 3, 7 and 6 shared KO IDs. This suggests that these three species are not of great importance within the ecosystem and could likely be removed without disrupting the ecological balance.

The comparison of the almost unique KO IDs also revealed the importance of four species that are likely keystone species within the ecosystem. Raoultella sp. 6BF7, Bacteriodes ovatus 5MM, Escherichia coli 3FM41, and Parabacteroides distasonis 5FM all had high levels of almost unique pathway, the majority of which were shared between these four species. Raoultella sp. 6BF7 and Escherichia coli 3FM41 in particular shared an unusually high number of KO IDs when looking at KO ID shared by two species. When examining the KO IDs shared by four species Bacteriodes ovatus 5MM and Parabacteroides distasonis 5FM shared a high number of KO IDs with Raoultella sp. 6BF7 and Escherichia coli 3FM41. This suggests that these four species may interact and play key roles in the ecosystem. Several species were also identified with low levels of almost unique pathways, having three or less KO IDs shared for the comparisons of two, three or four species (Table 5). Faecalibacterum prausnitzii 40FAA, Lachnospira pectinoshiza 34FAA, and Eubacterium rectale 29FAA had low levels of shared KO IDs in all three of the comparisons. Collinsella aerofaciens, and Dorea longicatena 42FAA also had low KO IDs in two of the three comparisons. This suggests that these five species may not play any major role in necessary low-level redundancy.

Table 5 is a summary of a comparison of KEGG orthology assignments shared by two, three or four species. Table 5 summarizes the species found to have low levels of almost unique pathways, having three or less KO IDs shared for between two, three or four species. Species highlighted in bold text fall into this category for two or more comparisons. Numbers in brackets indicate the number of KO IDs shared (prior to conflict resolution).

TABLE 5

| Two Species | Three Species | Four Species |
|---|---|---|
| [symbol] 40FAA (2) | [symbol] 40FAA (2) | [symbol] 40FAA (2) |
| [symbol] 34FAA (2) | [symbol] 34FAA (3) | [symbol] 34FAA (2) |
| [symbol] 29FAA (3) | [symbol] 29FAA (1) | [symbol] 29FAA (3) |
| [symbol] (3) | [symbol] (3) | — |
| [symbol] 42FAA (3) | — | [symbol] 42FAA (3) |
| Ruminococcus torques 30FAA (3) | Roseburia faecalis 39FAA (1) | — |
| Clostridium sp. 21FAA (3) | Bifidobacterium adolescentis 11FAA (2) | — |
| Eubacterium desmolans 48FAA (3) | Roseburia intestinalis 31FAA (3) | — |
| Eubacterium ventriosum 47FAA (3) | Eubacterium eligens F1FAA (2) | — |

The final pathway analysis resulted in only twenty-two of the thirty-three initial bacteria having unique pathways not covered by any other bacteria within the RePOOPulate system. A list of the final twenty-two species included in the updated model can be found in Table 4. The KEGG pathway map showing the unique pathways for these twenty-two key species can be seen in FIGS. 7 and 8 and a chart listing the pathways that these KO IDs map to can be found in Table 12. The consideration of the number of nodes for each strain that are crossed by pathways unique to the strain allows for a better idea of the possible unique unknown pathways that are present, and by looking at the highest number of connected nodes we gain some idea of the relevance of the pathways, as the higher the number of connected nodes, the higher the likelihood of importance of the pathway. An examination of this data showed, both Bacteriodes ovatus 5MM and Lachnospira pectinoshiza 34FAA have a higher numbers of unique nodes than most of the other species (12 and 8 respectively), however the highest number of connected nodes is only 2 for both. This suggests there may be unknown pathways involved. The most relevant species appears to be Raoultella sp. 6BF7, which has 46 unique nodes with the highest number of connected pathways being 15. This is five times greater the species with the next highest number of connected nodes, Roseburia intestinalis 31FAA, which has 3 unique nodes all connected (Table 11).

A comparison of the final list of KO IDs for the twenty-two key species compared to the list of KO IDs for the original thirty-three species revealed a loss of two KO IDs (K07768 and K11695) resulting from the removal of the eight species strains found to be redundant in Part I. The first KO ID was likely lost as a result of the removal of Eubacterium rectale 18FAA. This was the only bacterial species or strain that appeared to have had an error occur in genome assembly, having an overly large number of contigs for a relatively small genome size (FIG. 3C). Further research is required to determine the true importance of this strain. The KO ID that appears to have been lost (K07768) maps to three regulatory pathways within the two-component system for signal transduction, however two of those pathways are also mapped by another KO ID (K07776), which is still present in the final list of KO IDs for the twenty-two species ecosystem. This suggests that only a single small pathway was lost, which would likely not affect the ecological balance. The second KO ID (K11695) lost in the process of redundancy removal maps to a single metabolic pathway for peptidoglycan biosynthesis and is the only KO ID that maps to this pathway. This KO ID was lost as a result of the removal of Bifidobacterium longum 4FM. It is unclear whether the loss of this pathway will have a negative effect on the ecosystem's sustainability and further study is required to determine whether this bacterial strain may be necessary.

A closer look at the unique pathways for the twenty-two species suggests that further optimization of the number of species may be possible. The map showing the unique pathways revealed four bacterial strains with very few unique pathways including: Eubacterium desmolans 48FAA, Faecalibacterum prausnitzii 40FAA, Ruminococcus species (strain A) and Ruminococcus sp. 11FM, each of which only maps to a single map element and only one or two pathways (Table 12). This evidence combined with the information gained from comparing the pathways shared by two, three and four species (Table 5) suggests that Eubacterium desmolans 48FAA and Faecalibacterum prausnitzii 40FAA could likely be removed without causing imbalance in the ecosystem. Lachnospira pectinoshiza 34FAA and Collinsella aerofaciens also showed very few almost unique pathways (Table 5) and only have a few unique KO IDs and pathway elements (Table 12; 3 KO IDS, 6 elements and 2 KO IDs 2 elements, respectively). Further research would be required to determine the necessity of these four species in order to justify their removal or inclusion in a new prototype RePOOPulate ecosystem.

### Part III: Comparison of KEGG Pathway Coverage

### Methods

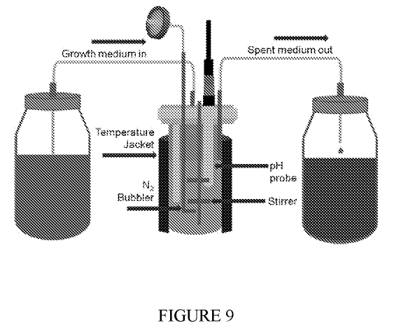

The list of KO IDs for all thirty-three species with weights determined by number of KO ID replicates within the RePOOPulate ecosystem created in Part II was loaded into ipath2.0 and used to create a custom map with lines colored in blue and weights determined by the number of replicates for each KO ID. Conflicts in weight were resolved using the automatic method used by iPath2.0 of randomly choosing between conflicting weights. The same process was completed for the list of KO IDs and updated weights for the optimized ecosystem consisting of the twenty-two species with unique KO IDs; lines for this map were colored black. The “healthy” human gut microbiome for comparison was taken from a study by Kurokawa et al., which is herein incorporated by reference in its entirety, and a completed list of KO IDs with weights is provided on the iPath website. The goal of the Kurokawa et al. study was to identify common and variable genomic features of the human gut microbiome. The study comprised of large-scale comparative metagenomic analyses of fecal samples from 13 healthy Japanese individuals of various ages, including unweaned infants. The data from this study had been previous used in the development of iPath2.0 as a demonstration of its capabilities and was chosen for this comparison because of the ease of use under the time limitations. iPath2.0 maps for the Kurokawa et al. data were created using the custom map function and the provided list. The lines for this list are colored red. The custom maps for all three data sets were then downloaded in portable document format (PDF).

[000316] The three PDF images were loaded into GIMP 2.8.10 (GNU image manipulationprogram) as separate layers and the transparency was manipulated by coloring to alpha channel such that the Kurokawa et al. data and both sets of RePOOPulate pathways could be visualized. This was done in order to visually compare how well each of the RePOOPulate ecosystems matched an example of the natural human gut microbiome, as well as each other, to determine the coverage of the KEGG pathways (FIG. 9). The three lists of KEGG IDs (one for each map), as well as the list of unique KEGG IDs found in Part II were also compared using a Microsoft Excel spreadsheet table. In order to optimize this process the Kurokawa et al. KO IDs were matched to the internal iPath list to remove any KO IDs that did not map to iPath2.0 pathways in the same way that the other lists were matched in Part II.

FIG. 9 shows a comparison of the RePOOPulate data to a healthy microbiome. A) Metabolic pathway map comparing the full RePOOPulate community before and after optimization to data from the Kurokawa et al. study. B) Regulatory pathway map comparing the full RePOOPulate community before and after optimization to data from the Kurokawa et al. study. Red lines represent the Kurokawa et al. data, blue lines represent the original RePOOPulate data with all 33 genomes included and black lines represent the optimized RePOOPulate data with only 21 genomes included.

Results

The matched list of KO IDs for the full thirty-three species RePOOPulate ecosystem was compared to the matched list of Kurokawa et al. KO IDs, which revealed 635 KO IDs found in the RePOOPulate data set, which are not in the Kurokawa et al. data, and 86 KO IDs found in the Kurokawa et al. data but not in RePOOPulate. The two KO IDs removed during the optimization process were not in the Kurokawa et al. data set. Of the KO IDs unique to either the Kurokawa et al. data or RePOOPulate 63 KO IDs had pathways that were shared with unique pathways from the other data set. 27 unique KO IDs for the Kurokawa et al. data had at least one overlapping pathway with the unique KO IDs for RePOOPulate, and 36 unique RePOOPulate KO IDs had at least one pathway shared by the unique KO IDs from the Kurokawa data. Further analysis is required to more closely examine the exact pathways missing from the RePOOPulate ecosystem that should be present in order to maintain a healthy gut microbiome.

The list of KO IDs that were unique to a single species within the twenty-two species of the optimized ecosystem was also compared to the matched Kurokawa et al. data set. Of the 117 unique KO IDs identified only 14 were also in the Kurokawa et al. data, these are highlighted in blue in Table 12. The 14 KO IDs that were unique to a single species and matched the Kurokawa et al. data were found in only nine species, suggesting these species may be the most important in the ecosystem (see Table 4).

A visual comparison of the two RePOOPulate versions with either thirty-three or twenty-two species revealed only small differences in the number of replicates of KO IDs with no obvious loss of data (FIG. 9). A visual comparison of the RePOOPulate data and the Kurokawa et al. data revealed some obvious gaps in the number of replicates of a few metabolic pathways in the RePOOPulate data when compared to the Kurokawa et al. data. This is likely do to a much larger number of bacteria present since the majority of these occurrences was in the area metabolism necessary for life, and would therefore be present in all bacterial species and would have a higher number of replicates for a larger variety of species. There are also several areas within the regulatory pathways map that appear to have an under abundance or absence of coverage in the RePOOPulate ecosystem. These include areas of the aminoacyl-tRNA biosynthesis pathways, ABC transporter pathways, two-component system and bacterial secretion system in particular. Further work would be necessary to understand the importance of these missing elements in order to ascertain whether the RePOOPulate system requires further modification to incorporate species that are able to regulate the pathways.

### Discussion

The goal of this study was to elucidate new information about the potential health benefits of novel maize starches produced through mutations in the starch biosynthesis pathway. Starch analysis included evaluation of the RS content and the effects on in vitro fermentation by human fecal microbiota, to determine potential prebiotic properties of the starch substrates. Chemostat-cultured microbial communities seeded from fecal inocula were shown to be a useful, reproducible inoculum for small-scale batch fermentations. In vitro fermentation resulted in unique changes to the fecal microbiota depending on the starch substrate, and these changes were shown to be different between fecal donors. Differential production of metabolites was observed in fermentation profiles from vessels seeded with material from different donors; however, using material from the same donor, metabolite profiles did not change appreciably in response to the different starch substrates.

Digestions

The six maize lines used in this study were selected for differences in their starch structure due to mutations in the starch biosynthesis pathway that resulted in modified amylose:amylopectin ratios; this in turn has been proven to affect the quantity of RS. RS determinations utilizing the Megazyme resistant starch assay kit revealed Cg102ae1-ref, Cg102ae1-Elmore and Cgx333Su2 contained the greatest quantities of resistant starch both before and after in vitro digestion, while Cg102wx contained the least. This was expected as the mutations in the starch biosynthesis pathways of the first 3 maize lines result in modifications to the starch structure that increase the RS content while the opposite is true for Cg102wx. In most cases RS, SS and TS contents of the starch substrates decreased after the in vitro digestion which is to be expected as cooking gelatinizes the starches making them more susceptible to digestion. RS is not gelatinized during most cooking applications such as boiling and baking. However, autoclaving reaches much higher temperatures and pressures than conventional cooking and whilst autoclaving provides sterilization of the starch substrates for subsequent use in fermentations, the process may have resulted in partial gelatinization of the RS fraction. Interestingly the RS content of Cgx333Su2 increased after the digestion procedure. The substantial differences in the genetic backgrounds of the maize lines Cg102 and Cgx333could be responsible in part for the different responses to the digestion procedure resulting in increased RS for Cgx333Su2.

A sterile starch substrate in fermentation experiments was an initial goal such that starch fermentation by the gut microbiota would not be influenced by environmental microbes associated with the maize kernels. Thus, steps were taken to produce a sterile starch substrate, but all samples were found to have some level of contamination. However, this contamination was found not to influence the small-scale batch fermentations. Starch substrate controls at 0 h and 48 h showed no changes in the DGGE profiles (FIG. 3.1). The starch substrates were prepared and digested aerobically, and as such the contaminants may have been strict aerobes that were unable to survive in the anaerobic environment used for gut microbial fermentations. This may explain why previous studies have not paid much attention to maintaining sterility during pre-digestion protocols. Since ensuring sterility of starch substrates does not appear to be critical to assessment of fermentations by the gut microbiota, boiling rather than autoclaving starches should be adequate for this type of work, and has the benefit of more closely resembling the everyday cooking process for starches, providing a more physiologically relevant substrate. Furthermore, as RS displays a dose-dependent response for the production of SCFA, the isolation of pure starch may be optimal as opposed to a cornmeal.

Small Scale Batch Fermentation Reproducibility

Chemostats can be used to reproducibly develop and maintain complex communities originating from human fecal samples. We used three separate runs inoculated with three different donor's feces as stable inoculum sources for studying the fermentation profiles of the 6 starch substrates in small scale batch fermentation models. To our knowledge this is the first instance where a stable chemostat model was used as an inoculum source for batch fermentations. This method provides advantages over repeated fecal collections from donors as chemostats provide a consistent community over a prolonged period of time and can be sampled when needed. In comparison, repeated fecal donations give rise to temporal shifts in the fecal microbiota due to the presence of transient species within the gut. Finally, an in vivo to in vitro transition occurs when culturing fecal communities, changes observed in batch fermentations may be misconstrued by this transition. The use of stable chemostat cultures, where this transition has already taken place, makes comparisons between replicate fermentations simpler, as variation in the community can be directly attributed to the effects of the treatment.

The in vitro chemostat model used in these experiments has been previously validated. While it does not give rise to fecal communities that are identical to the inoculum material, the communities are nevertheless stable and diverse communities that are largely representative of those found in vivo, and can be used for experimentation. In this work, the microbial community structure and the dynamics of the chemostat runs and small scale batch fermentations were analyzed using DGGE, a molecular fingerprinting technique. In addition, metabolic changes within batch fermentations were analyzed with the use of SPME GC-MS.

As this was the first study using chemostat cultures as an inoculum source for batch fermentations as opposed to fresh fecal samples, our first aim was to validate the reproducibility of the fermentations. Fermentations resulted in nearly identical community compositions between technical replicates. The reproducibility between biological replicates was dependent on the stability of the chemostat vessel used. The chemostat run seeded with feces from donor 9 achieved steady state prior to sampling and maintained low rate of change values throughout the entirety of the sampling period (FIG. 12c). As such, replicate fermentations had high % SI indicating identical community profiles for all 6 starch substrates tested, immediately following inoculation. Similar results were obtained for batch fermentations with inoculum originating from the chemostat seeded with feces from donor 5. Furthermore, fermentations with fecal inocula from both donor 5 and 9 progressed in a reproducible manner with replicates maintaining identical community dynamics after 48 h. This work demonstrated that it is, however, an absolute requirement that chemostats reach a steady state prior to sampling for use as an inoculum source in batch fermentations to obtain reproducible results. Sampling of the chemostat vessel seeded with fecal microbiota originating from donor 2, in contrast to those for donors 5 and 9, was initiated during a period in which there was still a rapid rate of change within the vessel (FIG. 12a). High % SI values, indicating identical communities, were observed at both oh and 48 h within technical replicates but not between biological replicates, indicating dissimilarity between inocula. Thus it can be concluded that steady state chemostats can be used to inoculate small scale batch fermentations with a high degree of reproducibility in order to study the effects various substrates have on the microbiota. This method may also be able to detect small changes that may be missed using traditional batch fermentation methods because of the significant community changes that take place during the in vivo to in vitro transition.

Responses to Starch Substrates

Cluster tree analysis and NMDS of DGGE profile similarities resulted in clustering of starch substrates into 3 groups following fermentation by donor 9′s fecal microbiota, while only two groups were evident for fermentations with the fecal microbiota from donor 5. No conclusions could be made concerning the fermentations with fecal microbiota from donor 2 because steady state had not been attained in the chemostat vessel prior to sampling, thus these results will not be discussed further.

Samples as a result of fermentation of Cgx333 and Cgx333 Su2 clustered together and separately from all other starch substrate fermentations with fecal microbiota from both donors 5 and 9, indicating that Cgx333 and Cgx333Su2 were fermented differently from the other starches. Although Cgx333Su2 contained increased quantities of RS compared to the wild-type (Cgx333), both imparted similar effects on the microbial communities derived from donors 5 and 9. This suggested that the mutation in Cgx333Su2 (which causes reduced amylopectin synthesis) did not impart a greater prebiotic effect on the fecal community per se. Starch derived from Cg102ae1-ref and Cg102ae1-Elmore lines contain longer amylopectin chains with reduced branching, resembling the structure of amylose thus increasing the RS content. These starch substrates had a pronounced effect on the community dynamics different from the wild type Cg102, resulting in unique community profiles. Collectively with the effects observed from all of the fermentations, the results support the hypothesis that different mutations alter the fermentation properties of the starch substrates stimulating different groups of colonic bacteria. Similar studies have assessed changes to the fecal microbiota in responses to starch substrates with modified structures; however our study took a more holistic approach by analyzing community level changes. In contrast most other studies examine only a subset of well-characterized probiotic bacterial species, thus missing other significant changes only seen when the whole fecal community is assessed.

For example, two RS polymorphs produced through differential processing of high amylose maize starch (HAMS) were shown to induce unique ecological shifts in fecal communities after 24 hours of fermentation. One polymorph resulted in increases in Bacteroides spp. and Atopobium spp., while the other polymorph stimulated growth of Bifidobacterium spp. A rat model was used to observe changes to the fecal microbiota in response to diets supplemented with two distinctive low amylose maize starches (LAMS), HAMS or butyrylated HAMS (HAMSB). These authors reported that both high RS diets independently resulted in unique changes to the microbiota while no difference was seen between the two LAMS. The HAMS diet induced increases of Ruminococcus bromii-like bacteria, while the HAMSB diet increased populations of Lactobacillus gasseri and Parabacteroides distasonis.

Additionally, unique changes to the gut microbiota between the starch substrates were observed for fermentations using fecal microbiota obtained from donor 9, and to a lesser degree with donor 5. This suggests that the initial composition of an individual's microbiota has a significant impact on the effects of a given substrate and supports the hypothesis that individual's gut microbiota will respond differently to the novel maize starches. Similar results were reported with individual responses between 10 subjects consuming RS enriched crackers. Of the taxa identified as significantly affected by the consumption of RS, none displayed a similar response in all 10 subjects. These varied responses could be due to several factors such as strain-level differences in substrate utilization, or the specific abundance, or absence, of particular species in a given individual's gut. As well, host physiological factors play a significant role in shaping an individual's gut microbiota. For example varied gut transit times, digestion rates, and pH all influence the colonic environment and the microbiota therein. Better understanding of these inter-individual differences in the gut microbiota will be pivotal for tailoring prebiotics for personalized health.

Metabolite Production

Fermentation of dietary fiber has been consistently reported to increase the production of SCFAs, which in turn have a significant influence on the overall health of the host. 90-95% of the total SCFAs that are produced within the human colon via fermentation of carbohydrates are acetate, propionate, and butyrate, as such, studies of RS and other dietary fibers routinely use a targeted approach to study changes in these metabolites. We utilized an untargeted metabolomic approach in this study that has previously been used to identify differences in fecal VOC between individuals with both healthy and diseased (dysbiotic) guts. This untargeted approach was used to capture a large number of metabolites in a given sample in hopes of identifying novel biomarkers, as well as SCFAs, resulting from the fermentation of the starch substrates.

We observed a consistent increase in the production of butanoic acid (butyrate) for all starch substrates fermented by the fecal microbiota derived from all donors. Interestingly acetate was not detected in any of the samples, which was surprising as other studies report this in the highest quantities compared to other SCFA. However, it has been shown that the production of butyrate is dependent on acetate, with ˜80% of butyrate production attributed to extracellular conversion of acetate through the butyryl CoA:acetyl CoA transferase pathway. As only the 48 h time points were analyzed for changes in SCFA production it is possible that much of the acetate in these samples had been converted to butyrate by the microbiota. Thus it may be necessary to look at earlier time points to elucidate the kinetics of the production and depletion of acetate.

The production of both propanoic acid and pentanoic acid was detected only in the fermentations with the fecal microbiota from donor 9. It has been proposed that the inter-individual variation of the fecal microbiota results in different functional capabilities, which could be a plausible explanation as to why the 3 fecal donors used in this study resulted in the production of different collections of metabolites. Similar results have been reported previously; a study indicated that obese mice have an increased capacity for energy harvest from foods, which is linked to the composition of their fecal microbiota. Obesity may result from an increased fermentative capacity producing SCFAs, which are absorbed by the host and used as an energy source. This could explain some of the different results observed between the 3 fecal donors in terms of metabolites produced.

In this study, despite using a technique to enable detection of multiple metabolites, no unique, previously unreported metabolites of fermentation were detected. However the results observed supported the hypothesis, as unique fermentation profiles were produced between individuals. Furthermore differences in the metabolites produced between the starch substrates appeared to exist as determined by PCA, but could not be confirmed with a statistically significant OPLS-DA model. Because of this, future work to determine the fermentation profiles of microbiota samples in response to different starch substrates may benefit from analysis using simpler (though less inclusive) targeted metabolomic techniques. Targeted approaches may also give more quantifiable results and allow greater elucidation of significant differences between the metabolites produced by the fermentation of different starch substrates; the untargeted analysis using GC-MS in this study did not detect any such differences.

PhAST Blue

DGGE analysis of DNA originating from small scale batch fermentations of fecal communities derived from stable, single-stage chemostat cultures revealed a considerable change to the community dynamics after 48 hours of fermentation. As batch fermentations are closed systems, the changes observed may have been skewed by the amplification of DNA originating from dead cells. One way to solve this issue is through the use of differential amplification of DNA from live cells. The use of ethidium monoazide (EMA) to treat environmental samples prior to PCR amplification prevents amplification of extracellular DNA in the sample, as well as DNA from cells that are dying and thus permeable to EMA uptake, whereas DNA in live cells is protected from the chemical. The PhAST Blue kit became available for use near the end of the study as such it was only briefly evaluated to determine the reproducibility of this technique as a means of treating fecal communities prior to molecular analysis. In this study, EMA treatment prior to PCR amplification showed a consistent signal reduction in some bands and an increase in intensity of others on DGGE gels compared to that of untreated samples, while still maintaining the degree of similarity between replicates (FIG. 21). Therefore, suggesting that this method is a reliable and reproducible method for labeling DNA originating from dead cells. This parallels the results observed by another study analyzing a mature biofilm from a water reservoir. The authors of this paper mention however, that some caution must be taken when analyzing these results, as more studies must be completed to ensure that this procedure can be applied to a wide range of microbial species without introducing bias. EMA treatment may, however, prove an invaluable method in microbial ecology, improving the sensitivity of molecular techniques when analyzing changes in microbial communities by providing a simple pre-treatment to target only the viable cell populations. Future studies should incorporate the use of this technique in the analysis of both short-term batch fermentations and long-term chemostat studies as it may more accurately reveal the community level changes occurring within the vessels.

Chemostat Feeding trial

Twin-vessel, single-stage chemostats mimicking the distal colon have been shown to be an effective means of reproducibly studying perturbations in the gut microbiota in response to various stressors. Fermentation properties of prebiotic substrates have been studied using various continuous culture models. Many of these experiments however lacked control vessels to ensure observed changes were not due to the adaptation of the community to the in vitro model (the vessel baseline was used as its own control), or failed to establish an adequate steady state community prior to experimentation. In this study we aimed to confirm the use of twin-vessel, single-stage chemostats seeded with fecal microbial communities as an alternative to complex human feeding trials. To our knowledge this is the first instance where a predigested substrate was used to supplement the medium of an in vitro model to mimic an in vivo feeding trial.

The twin-vessel, single-stage chemostats displayed community changes for fecal communities from both donors (5 and 9) following initiation of the modified media (RS+ and CS+). The % SI of the twin-vessels dropped over the course of the simulated feeding trial indicating that the two starch substrates had different effects on the fecal communities. Upon returning to a basal medium feed, the % SI of the twin-vessels began to increase, potentially indicating that the vessels were returning to a basal state as at the initiation of the feeding trial.

The low similarity seen between the twin-vessels inoculated with donor 5 feces could have been due to divergence in the communities during establishment of steady state, as it was observed that V5-2 required the addition of base at a much higher rate than V5-1 throughout the course of the run. However, the trends observed with the community dynamics parallel those seen with the twin-vessels inoculated with fecal microbiota from donor 9, despite the differences in the steady state communities.

The use of twin vessel, single stage chemostats was found to have distinct advantages over more traditional batch culture fermentations, because the former enable fecal communities to transition to a stable in vitro state prior to exposure to the substrate being tested. Furthermore twin vessel, single-stage chemostats enable one to study the effects of varying substrate quantities and treatment time periods, as opposed to batch cultures, which have only a short experimental window. As such, twin vessel, single-stage chemostats can be effectively used for controlled experiments to investigate the effect of feeding prebiotics or introducing other perturbations to the gut microbial ecosystem independent of the host.

Conclusions and Additional Embodiments

RS is a proven prebiotic with a significant potential to improve human health through the modulation of the fecal microbiota. Numerous forms of RS originating from a wide range of starch-rich foods have been shown to have varied prebiotic effects both within an individual's fecal microbiota, and between the microbiota of different individuals. The study completed here provides the groundwork for screening and identifying modified starch substrates with increased prebiotic potential. DGGE clearly discriminated community profile changes between the starch substrates. Although SPME GC-MS was used in an attempt to widen the spectrum of metabolites that could be detected, only increases in SCFA (particularly butyrate) were consistently observed. This indicates that future attempts to determine differences between starch substrates should measure changes in metabolites with a particular focus on SCFAs and may be better accomplished using quantitative targeted metabolic approaches, opposed to untargeted methods.

Additional Embodiments

Further evaluation of the prebiotic potential of the modified starches will be examined, in particular Cg102ae1-ref and Cg102ae1-Elmore because these lines contain the greatest quantities of RS and appeared to have the greatest effect on the fecal microbiota. Administering Cg102ae1-ref and Cg102ae1-Elmore to patients in need thereof can promote growth of at least one bacterial strain in the gut microbiome.

Additionally, the use of 16S rRNA community profiling can be used to elucidate community compositional changes that are occurring in response to the starch substrates. This will aid in further identifying substrates with a greater prebiotic potential in terms of enriching taxa with biochemical processes that are associated with beneficial effects both on the host and the nascent microbial community. Furthermore, inter-individual responses of fecal communities from several individuals, covering a wide range of dietary lifestyles, could better characterize community structures predisposed to optimal utilization of given starch substrates.

Another possible avenue to explore could be how these prebiotics affect individuals with dysbiotic guts, such as those suffering with IBD or ulcerative colitis. Since ‘dysbiosis’ is a poorly defined term describing a situation that is not well understood in terms of microbial ecology, the methods developed in this work could contribute to a better appreciation of the underlying mechanisms of dysbiosis in terms of inability for a given ecosystem to utilize substrates effectively.

Although the models described in this work simulate the in vivo environment, they cannot easily model host responses. Future studies in humans or animal models will be performed to confirm the prebiotic nature of potentially prebiotic substrates. However, fecal batch fermentations and chemostat models can be used to screen candidate starches and other substrates for their prebiotic potential in a cost-effective manner. Continued research into the factors defining a person's fecalm microbiota and their functional capacities along with an increased understanding of starch biosynthesis and factors influencing digestibility will continue to propel this work forward leading to a new era of personalized health and nutrition.

Bacterial Communities

There are several limitations to the study design outlined in this report. One of the major sources of possible error is the high level of manual manipulation of the data sets, which lends itself to the introduction of human error. The methods chosen to resolve conflicts and sort data were not ideal; in the future a more automated, programming-based approach would eliminate many of these possible sources of error and increase the validity of the results.

A second major issue in the design of this study is the general lack of knowledge about the metabolic and biochemical pathways of bacteria. The issue of possible important unknown bacterial pathways lends itself to an inability to correctly identify important species and the misidentification of redundancy. An attempt was made to correct for this error source through an examination of both the nodes and pathways in the analysis, however this does not account for all possible unknowns. Similarly, the use of the program iPath2.0 also introduces a certain element of the unknown since the program does not include all possible pathways or account for all known KEGG orthology assignments. The comparison of KEGG orthology assignments in this project focused solely on those used within the iPath2.0 program, both for simplicity and ease of understanding. However, this meant that of the 4210 KO IDs identified in the thirty-three genomes of the RePOOPulate ecosystem only 1536 were included in comparisons, leaving 2674 KO IDs unexplored in this analysis.

Accordingly, when our understanding improves regarding the metabolic and biochemical pathways of bacteria, this information regarding these pathways will be incorporated into the embodiments of the subject invention.

The analysis outlined in Part II of this report revealed only twenty-two of the thirty-three original strains of bacteria map to unique pathways. This suggests that some or all of these species may be the “keystone” species within the ecosystem and that the other species could possibly be redundant. This analysis does not account for the fact that a certain level of redundancy within the ecosystem may be required, certain bacterial interactions not examined may be ecologically necessary, or unknown bacterial pathways may play a role in the ecological balance of the community. It must also be mentioned that only nine of these species had unique KO IDs also found in the example of a “healthy” microbial community. Further work is required to definitively define the “keystone” species and pathways necessary for balance within the ecosystem of the human gut.

The final comparison in search of redundancies within the RePOOPulate ecosystem was designed to look at a natural “healthy” human gut bacterial population compared to the artificial community of the RePOOPulate project. This proved to be a challenge since a “healthy” bacterial population has yet to be clearly defined. This study data chosen to represent a “healthy” human gut microbiome was chosen because of time limitations; the data was readily available and already in the correct format for the pathway analysis program used in this study. However, the source of data was not ideal since it contained data on only 13 individuals, all of Japanese ancestry, and also included data on unweaned infants, which could be a source of error because of the dynamic nature of the gut microbiome at early stages of development. The fact that all fecal samples were from Japanese individuals could also be a source of error in the data, due to both a lack of diversity across human subjects and the unique diet of the Japanese. Previous studies have shown that the Japanese have a higher abundance of genes derived from marine bacteria do to the high levels of seaweed in the Japanese diet and a requirement for gut bacteria to breakdown this food source. These introduced marine bacterial genes could affect the pathways seen in the data set. If time had allowed a better source of data would have been the Human Microbiome Project or the European initiative MetaHit, which would have provided a source of data more typical of the North American gut microbiome.

### EXAMPLE

### Creation of a Bacterial Community

The next steps in the process of optimizing the RePOOPulate ecosystem involve the actual creation of the suggested bacterial community, in culture, to see if ecological balance is preserved with the removal of the apparently redundant species and strains. The metagenomic approach used in this study cannot tell us whether the identified genes are expressed and at what levels, therefore the actual functional activity of the community should also be examined through a metatranscriptomic approach. Metatranscriptomics uses messenger RNA isolated from the community that has been converted to complementary DNA and sequenced on a high-throughput platform. This approach allows for the characterization gene expression in the microbial ecosystem and would give a greater understanding of the interactions of the community as a whole. Accordingly, upon creating such a bacterial community, the bacterial community will be administered to a patient suffering from a dysbiosis (e.g., but not limited to, IBD, IBS, UC, cancer-related dysbiosis, etc.), and the patient will exhibit an improved gastrointestinal pathology.

### Conclusions

The evidence outlined in Part I of this study clearly shows redundancy in five of the six species examined. The evidence outlined in Part II is less clear, but there is some indication that several further redundant species can be found within the RePOOPulate ecosystem. The final analysis in Part III indicates that the RePOOPulate community is very close to emulating the metabolic and regulatory pathways of a healthy human gut microbiome. This comparison also indicates that an ecosystem consisting of twenty-two species rather than the original thirty-three would likely result in a more economic artificial bacterial community without loss of functionality or ecological balance. Further study with bacterial culture is required to test this theory.

What is claimed is:

1. A method of enriching at least one bacterial species from a target bacterial system, comprising:
culturing the target bacterial ecosystem in a culture media in a single-stage chemostat under the following conditions: (i) a system retention time of about 5 to about 290 hours, (ii) a temperature of about 37° C., (iii) a pH of about 6.8 to 7, and (iv) maintenance of anaerobic conditions to the chemostat for a time sufficient to enrich the at least one bacterial species;
wherein the culture media comprises a prepared starch substrate, and
wherein the target bacterial system is a fecal derived sample obtained from a patient that has not been treated with an antibiotic for at least 6 months.

2. The method of claim 1, wherein the prepared starch substrate comprises: a maize substrate, a corn substrate, a wheat substrate, a barley substrate, a legume substrate, an oat substrate, or any combination thereof.

3. The method of claim 1, wherein the prepared starch substrate is a maize substrate.

4. The method of claim 1, wherein the at least one bacterial species comprises: a Bacteroides spp., an Atopobium spp., Ruminococcus bromii, Lactobacillus gasseri, and Parabacteroides distasonis.

5. The method of claim 1, wherein the patient has not been treated with an antibiotic for at least 1 year.

6. The method of claim 1, wherein the system retention time is between about 20 to 70 hours.
